SEQUENCE LISTING (76 sequences)

SEQ ID NO: 1 - DNA, 29 bp, Artificial Sequence
  Synthetically Generated Sequence
ggcttaggcatctcctatggcaggaagaa

SEQ ID NO: 2 - DNA, 29 bp, Artificial Sequence
  Synthetically Generated Sequence
tagcccttccagtccccccttttctttta

SEQ ID NO: 3 - protein, 34 aa, Artificial Sequence
  Synthetically Generated Sequence
WMEWDREINNYTSLIHSLIEESQNQQEKNEQELL

SEQ ID NO: 4 - protein, 39 aa, Human immunodeficiency virus
DQQLLGIWGCSGKLICTTAVPWNASWSNKTLDMIWNNMT

SEQ ID NO: 5 - protein, 39 aa, Human immunodeficiency virus
DQQLLGIWGCSGKLICTTAVPWNASWSNKSLEMIWDNMT

SEQ ID NO: 6 - protein, 39 aa, Human immunodeficiency virus
DQQLLGIWGCSGKHICTTTVPWNSSWSNKTLDMIWNNMT

SEQ ID NO: 7 - protein, 39 aa, Human immunodeficiency virus
DQQLLGIWGCSGKHICTTNVPWNSSWSNKTLEMIWNNMT

SEQ ID NO: 8 - protein, 39 aa, Human immunodeficiency virus
DQQLLGIWGCSGKLICTTAVPWNTSWSNKTLDMIWNNMT

SEQ ID NO: 9 - protein, 39 aa, Human immunodeficiency virus
DQQLLGIWGCSGKLICTTTVPWNASWSNKTLDDIWNNMT

SEQ ID NO: 10 - protein, 39 aa, Human immunodeficiency virus
DQQLLGIWGCSGKLICTTNVPWNSSWSNKTQNMIWNNMT

SEQ ID NO: 11 - protein, 39 aa, Human immunodeficiency virus
DQQLLGIWGCSGKLICATTVPWNSSWSNKTQEMIWNNMT

SEQ ID NO: 12 - protein, 39 aa, Human immunodeficiency virus
DQQLLGIWGCSGKLICTTTVPWNSSWSNKTYNDIWNNMT

SEQ ID NO: 13 - protein, 39 aa, Human immunodeficiency virus
DQKFLGLWGCSGKIICTTAVPWNSTWSNRTFEMIWNNMT

SEQ ID NO: 14 - protein, 39 aa, Human immunodeficiency virus
DQQLLGIWGCSGKLICTTNVPWNTSWSNKTYNMIWNNMT

SEQ ID NO: 15 - protein, 39 aa, Human immunodeficiency virus
DQQLLGIWGCSGKLICTTNVPWNSSWSNKTLDMIWNNMT

SEQ ID NO: 16 - protein, 39 aa, Human immunodeficiency virus
DQQLLGLWGCSGKLICTTNVPWNSSWSNKTQDMIWNNMT

SEQ ID NO: 17 - protein, 39 aa, Human immunodeficiency virus
DQQLLGLWGCSGKLICTTNVPWNSSWSNKTQEMIWENMT

SEQ ID NO: 18 - protein, 39 aa, Human immunodeficiency virus
DQQLLGIWGCSGKLICTTNVPWNNSWSNKTQDMIWNNMT

SEQ ID NO: 19 - protein, 39 aa, Human immunodeficiency virus
DQQLLGIWGCSGKLICTTAVPWNSSWSNKTQEDIWNNMT

SEQ ID NO: 20 - protein, 39 aa, Human immunodeficiency virus
DQQLLGIWGCSGKLICTTAVPWNSSWSNKTQQMIWNNMT

SEQ ID NO: 21 - protein, 63 aa, Human immunodeficiency virus
CTDLRNVTNINNSSEGMRGEIKNCSFNITTSIRDKVKKDYALFYRLDVVPIDNDNTSYRLINC

SEQ ID NO: 22 - protein, 24 aa, Human immunodeficiency virus
QQLLGIWGCSGKLICTTAVPWNAS

SEQ ID NO: 23 - protein, 63 aa, Human immunodeficiency virus
CTDLRNATNTTSSSWETMEKGEIKNCSFNITTSIRDKVQKEYALFYNLDVVPIDNASYRLISC

SEQ ID NO: 24 - protein, 24 aa, Human immunodeficiency virus
QQLLGIWGCSGKLICTTTVPWNTS

SEQ ID NO: 25 - protein, 64 aa, Human immunodeficiency virus
CKDVNATNTTNDSEGTMERGEIKNCSFNITTSIRDEVQKEYALFYKLDVVPIDNNNTSYRLISC

SEQ ID NO: 26 - protein, 24 aa, Human immunodeficiency virus
QQLLGIWGCSGKLICTTAVPWNAS

SEQ ID NO: 27 - protein, 66 aa, Human immunodeficiency virus
CTDLKNDTNTNSSSGRMIMEKGEIKNCSFNISTSIRGKVQKEYAFFYKLDIIPIDNDTTSYSLTSC

SEQ ID NO: 28 - protein, 24 aa, Human immunodeficiency virus
QQLLGIWGCSGKLICTTAVPWNAS

SEQ ID NO: 29 - protein, 65 aa, Human immunodeficiency virus
CTNLKNATNTKSSNWKEMDRGEIKNCSFKVTTSIRNKMQKEYALFYKLDVVPIDNDNTSYKLINC

SEQ ID NO: 30 - protein, 24 aa, Human immunodeficiency virus
QQLLGIWGCSGKLICTTAVPWNAS

SEQ ID NO: 31 - protein, 62 aa, Human immunodeficiency virus
CTDVKRNNTSNDTNEGEMKNCSFNITTEIRDKKKQVHALFYKLDVVPIDDNNSNTSYRLINC

SEQ ID NO: 32 - protein, 24 aa, Human immunodeficiency virus
QQLLGIWGCSGKHICTTTVPWNSS

SEQ ID NO: 33 - protein, 60 aa, Human immunodeficiency virus
CSDVNATNSATNTVVAGMKNCSFNITTEIRDKKKQEYALFYKLDVVQIDGSNTSYRLINC

SEQ ID NO: 34 - protein, 24 aa, Human immunodeficiency virus
QQLLGIWGCSGKHICTTNVPWNSS

SEQ ID NO: 35 - protein, 64 aa, Human immunodeficiency virus
CTDLMNATNTNTTIIYRWRGEIKNCSFNITTSIRDKVQKEYALFYKLDVVPIDNDNTSYRLISC

SEQ ID NO: 36 - protein, 24 aa, Human immunodeficiency virus
QQLLGIWGCSGKLICTTAVPWNAS

SEQ ID NO: 37 - protein, 61 aa, Human immunodeficiency virus
CTDLKKNVTSTNTSSIKMMEMKNCSFNITTDLRDKVKKEYALFYKLDVVQIDNDSYRLISC

SEQ ID NO: 38 - protein, 24 aa, Human immunodeficiency virus
QQLLGIWGCSGKLICTTAVPWNTS

SEQ ID NO: 39 - protein, 64 aa, Human immunodeficiency virus
CTDFNNTTNNTTNTRNDGEGEIKNCSFNITTSLRDKIKKEYALFYNLDVVQMDNDNSSYRLTSC

SEQ ID NO: 40 - protein, 24 aa, Human immunodeficiency virus
QQLLGIWGCSGKLICTTTVPWNAS

SEQ ID NO: 41 - protein, 62 aa, Human immunodeficiency virus
CSNVNVTNNTTNTHEEEIKNCSFNMTTELRDKKQKVYSLFYRLDVVQINENNSNSSYRLINC

SEQ ID NO: 42 - protein, 24 aa, Human immunodeficiency virus
QQLLGIWGCSGKLICTTNVPWNSS

SEQ ID NO: 43 - protein, 60 aa, Human immunodeficiency virus
CSNANTTNNSTMEEIKNCSYNITTELRDKTQKVYSLFYKLDVVQLDESNKSEYYYRLINC

SEQ ID NO: 44 - protein, 24 aa, Human immunodeficiency virus
QQLLGIWGCSGKLICATTVPWNSS

SEQ ID NO: 45 - protein, 60 aa, Human immunodeficiency virus
CHNNITNSNTTNNNAGEIKNCSFNMTTELRDKKQKVYALFYRLDVVQINKNNSQYRLINC

SEQ ID NO: 46 - protein, 24 aa, Human immunodeficiency virus
QQLLGIWGCSGKLICTTTVPWNSS

SEQ ID NO: 47 - protein, 67 aa, Human immunodeficiency virus
CTNANLTNVNNITNVSNIIGNITNEVRNCSFNMTTELRDKKQKVHALFYKLDIVQIEDNNSYRLINC

SEQ ID NO: 48 - protein, 24 aa, Human immunodeficiency virus
QKFLGLWGCSGKIICTTAVPWNST

SEQ ID NO: 49 - protein, 62 aa, Human immunodeficiency virus
CTDVNVTNNNTNNTKKEIKNCSFNITTEIRDKKKKEYALFYRLDVVPINDNGNSSIYRLINC

SEQ ID NO: 50 - protein, 24 aa, Human immunodeficiency virus
QQLLGIWGCSGKLICTTNVPWNTS

SEQ ID NO: 51 - protein, 63 aa, Human immunodeficiency virus
CSNVNTTNATNSRFNMQEELTNCSFNVTTVIRDKQQKVHALFYRLDVVPIDDNNSYQYRLINC

SEQ ID NO: 52 - protein, 24 aa, Human immunodeficiency virus
QQLLGIWGCSGKLICTTNVPWNSS

SEQ ID NO: 53 - protein, 61 aa, Human immunodeficiency virus
CTDVNATNNDTNDNKTGAIQNCSFNMTTEVRDKKLKVHALFYKLDIVPISNNNSKYRLINC

SEQ ID NO: 54 - protein, 24 aa, Human immunodeficiency virus
QQLLGLWGCSGKLICTTNVPWNSS

SEQ ID NO: 55 - protein, 65 aa, Human immunodeficiency virus
CTDANATANATKEHPEGRAGAIQNCSFNMTTEVRDKQMKVQALFYRLDIVPISDNNSNEYRLINC

SEQ ID NO: 56 - protein, 24 aa, Human immunodeficiency virus
QQLLGLWGCSGKLICTTNVPWNSS

SEQ ID NO: 57 - protein, 59 aa, Human immunodeficiency virus
CTDVNVTINTTNVTLGEIKNCSFNITTEIKDKKKKEYALFYRLDVVPINNSIVYRLISC

SEQ ID NO: 58 - protein, 24 aa, Human immunodeficiency virus
QQLLGIWGCSGKLICTTNVPWNSS

SEQ ID NO: 59 - protein, 55 aa, Human immunodeficiency virus
CTNATNATNTMGEIKNCSFNITTELRDKKQKVYALFYRLDIVPLNENNSYRLINC

SEQ ID NO: 60 - protein, 24 aa, Human immunodeficiency virus
QQLLGIWGCSGKLICTTAVPWNSS

SEQ ID NO: 61 - protein, 71 aa, Human immunodeficiency virus
CTNVSSNGNGTYNETYNESVKEIKNCSFNATTLLRDRKKTVYALFYRLDIVPLNDENSGKNSSEYYRLINC

SEQ ID NO: 62 - protein, 24 aa, Human immunodeficiency virus
QQLLGIWGCSGKLICTTAVPWNSS

SEQ ID NO: 63 - protein, 65 aa, Human immunodeficiency virus; identical to SEQ ID NO: 29

SEQ ID NO: 64 - protein, 63 aa, Human immunodeficiency virus; identical to SEQ ID NO: 21

SEQ ID NO: 65 - protein, 64 aa, Human immunodeficiency virus; identical to SEQ ID NO: 25

SEQ ID NO: 66 - protein, 63 aa, Human immunodeficiency virus
CTDLRNATNITSSSWETMEKGEIKNCSFNITTSIRDKVQKEYALFYNLDVVPIDNASYRLISC

SEQ ID NO: 67 - protein, 60 aa, Human immunodeficiency virus
CHHNVTKITQEMKGEIKNCSFNVTTELRDKKQKVYSLFYRLDVVPFDNSDDSSPYRLINC

SEQ ID NO: 68 - protein, 61 aa, Human immunodeficiency virus
CHHNVTNITQDMTGEKNCSFNVTTSELRDKKQKEVYSLFYRLDVVPLKNSDNNSSYRLINC

SEQ ID NO: 69 - protein, 71 aa, Human immunodeficiency virus
CSNVTRNSSVTGNNTVEAGEEIKNCSFNMTTELRDKKKKVYSLFYRLDVVQIDQGNSSSSSNNNEYRLVNC

SEQ ID NO: 70 - protein, 73 aa, Human immunodeficiency virus
CSNVNVTRNNSSTGNDTVKAGEEIKNCSFNMTTELRDKTKKVYSLFYRLDIVQIDQGNSSSSSNNSEYRLVNC

SEQ ID NO: 71 - protein, 60 aa, Human immunodeficiency virus
CTNVNNTTNVNNNTGWDEERKNCSFNVTTELRDKRQKVYSLFYKLDVVQIDNSSYRLINC

SEQ ID NO: 72 - protein, 64 aa, Human immunodeficiency virus
CTDVNNTNVNNTTNGNNSMWDEERKNCSFNITTEIRDKRQKVYSLFYKLDVVQIDNSSYRLINC

SEQ ID NO: 73 - protein, 61 aa, Human immunodeficiency virus
CSNVNVTGNKVNITGREDMKNCSFNITTEIRDKKKQAYSLFYRLDIVPINSSTNEYRLINC

SEQ ID NO: 74 - protein, 83 aa, Human immunodeficiency virus
CSNVNVTDRDVNVTSNVTVSKDTVTDNKGNNTGWEDMKNCSFNITTERGDKKKAYSLFYRLDIVPIDSSTNNNSTNEYILINC

SEQ ID NO: 75 - protein, 71 aa, Human immunodeficiency virus
CTNLKKNDMKKNDTNATNISWGKMEEGEIKNCSFNVTTRRGNKMQKEYAFFYKLDVVPIDDDNTSYTLINC

SEQ ID NO: 76 - protein, 77 aa, Human immunodeficiency virus
CTNVKINDTNANNATKANNTTTNITIWEKMEEGEIKNCSFNVTTSIGNQMQKNYALFYRLDVVPMNDDNTSYTLINC

(19) United States
(12) Patent Application Publication
(10) Pub. No.: US 2016/0122395 A1
(43) Pub. Date: May 5, 2016

(54) Immunogenic compositions and a process for producing same

(71) Applicant: THE MACFARLANE BURNET INSTITUTE FOR MEDICAL RESEARCH AND PUBLIC HEALTH LTD, Melbourne, Victoria (AU)

(72) Inventors: Heidi DRUMMER, Melbourne (AU); Pantelis POUMBOURIOS, Melbourne (AU)

(73) Assignee: The MacFarlane Burnet Institute for Medical Research and Public Health Ltd.

(21) Appl. No.: 14/776,802

(22) PCT Filed: Mar. 17, 2014
(86) PCT No.: PCT/AU2014/000287

§ 371 Date: Sep. 15, 2015

Related U.S. Application Data
(60) Provisional application No. 61/852,179, filed on Mar. 15, 2013.

(51) Int. Cl. C07K 14/005; C12N 7/00; A61K 39/21

(52) U.S. Cl. CPC C07K 14/005; A61K 39/21; C12N 7/00; A61K 2039/5258

(57) ABSTRACT

The present invention provides a modified HIV envelope glycoprotein (Env) antigen or a lipid containing vehicle comprising same. The Env antigen comprises one of a second site suppressor mutation in residue 674 of the membrane proximal ectodomain region (MPER) of HIV gp41; a second site suppressor mutation which ablates a glycosylation site in the variable region (V1) region of gp120; or a second site suppressor mutation ablating a glycosylation site in the V1 region of gp120 and a second site suppressor mutation in residue 674 of the MPER of HIV gp41. It is preferred that the lipid containing vehicle is a HIVLP.

### RELATED APPLICATION

The present application is a U.S. national phase of PCT Application No. PCT/AU2014/000287 filed on 17 Mar. 2014, which claims priority from U.S. Provisional Patent Application No. 61/852,179 filed on 15 Mar. 2013, the disclosure of which is included herein by reference in its entirety.

### FIELD

The present specification teaches in the general field of pathogenic viruses. More particularly, the specification relates to human immunodeficiency virus (HIV) vaccines and related fields. In one particular aspect, the specification relates to modified HIV envelope glycoproteins (Env) and provides a process for modifying HIV Env-based immunogens for use in vaccine protocols to enhance the ability of a subject to produce broadly neutralizing antibodies (brNAbs).

### SEQUENCE LISTING

This application contains a Sequence Listing which is submitted herewith in electronically readable format. The electronic Sequence Listing file was created on Jan. 6, 2016, is named “376507_ST25.txt” and had a size of 35.9 KB. The entire contents of the Sequence Listing in the electronic “376507_ST25.txt” file are incorporated herein by this reference.

### BACKGROUND

Bibliographic details of references referred to by number in Example 1 and Example 2 are listed at the end of the Examples under “Bibliography 1” and “Bibliography 2”, respectively. Bibliography details of references referred to by author are listed at the end of the Examples as “Bibliography 3”.

Bibliography details of references referred to in Table A are listed under Table A.

The reference to any prior art is not and should not be taken as an acknowledgment or any form of suggestion that this prior art forms part of the common general knowledge.

There has long been an interest in the ability of certain host immunoglobulins (antibodies) to reduce or block (neutralize) the ability of viral pathogens to initiate or perpetuate an infection in the host. For example, in the case of hepatitis B virus, administration of anti-hepatitis-B virus antibodies within twelve hours of birth to infants born to infected mothers is effective in preventing mother to infant transmission of the virus. Coupled with an active HBV vaccine this strategy is 98% effective (Beasley et al., Lancet, 2: 1099-102, 1983). For HIV Rubrecht et al. (Rubrecht et al., Vaccine. 21: 3370-3, 2003) showed that passive transfer of neutralizing monoclonal antibodies that were available in 2003 (i.e., neutralizing monoclonal antibodies b12, 2G12, 2F5 and 4E10) was effective in protecting macaques against a clade B primary HIV isolate.

While it is possible for passive transfer of neutralizing antibodies to be an effective treatment or preventative approach, it is currently considered that the induction of a broad neutralizing antibody response will be a critical ability of any effective vaccine. A major perceived obstacle to a vaccine against HIV has been its high mutation rate leading to multiple different genetic forms of virus and more particularly enhancing the diversity within the Env glycoprotein which is the target of many neutralizing antibodies. Based upon genetic similarities, HIV-1 viruses (which cause infections in man more commonly than HIV-2) are grouped into four groups, M, O, N and P. M is the major group and this group has been classified into different geographically and genetically distinct clades, clades A through to H, J, and K.

There are examples of chronically infected patients who have over time developed serum antibodies to Env that neutralize virus from diverse HIV clades. Most of the early neutralizing antibodies identified display activity predominantly against neutralization sensitive HIV strains (tier 1 strains) while most circulating HIV strains are less sensitive to neutralizing antibodies (tier 2 strains). Thus, an effective vaccine is thought to be one which induces broadly neutralizing antibodies against multiple clades of HIV, or at least against clade C HIV which predominates world wide and has caused more than half of HIV infections. Furthermore, vaccines which engender antibodies that recognise tier 1 and tier 2 strains are also sought.

An effective HIV vaccine remains an elusive goal. HIV infections continue to cause millions of deaths around the world every year and since the HIV was recognised in 1990 as the causative agent of the AIDS epidemic, over 30 million people have died from AIDS-related causes. The World Health Organisation conservatively estimates that there were 34 million people living with HIV/AIDS in 2010 with 2.7 million people newly infected in that year of whom 14% were children. The mainstay of successful treatment is combination anti-retroviral drug therapy (cART) which can slow disease progression through viral suppression. However, cART but has serious side effects.

More recently, new, highly potent, and broadly neutralizing antibodies have been identified. The most powerful method for identifying neutralizing antibodies has been to sort individual memory B cells from rare individuals who display broadly neutralising antibodies (brNAbs) followed by micro neutralization assays. These antibodies are directed against the CD4 binding site, to the membrane proximal ectodomain region (MPER) of gp41 and to oligomannose glycan-dependent epitopes. The following table illustrates a range of neutralizing antibodies, their target sites and their neutralizing capacity.

TABLE A
Considerations for HIV-1 antibody vaccine

| | CD4bs | glycan | glycan | V1V2V3 | QNE | glycan | outer | gp41 | MPER |
|---|---|---|---|---|---|---|---|---|---|
| % Neut | B12¹ | VRC01¹ | 2G12¹ | PG9/16^{1,2} | PGT145¹ | PGT121/8^{1,3} | 2F5³ | 4E10³ | 10E8³ |
| Breadth | | | | | | | | | |
| IC50 < 1 μg/ml | 10 | 85 | 10 | 60 | 50 | 60 | 16 | 40 | 72 |

¹Walker et al., Nature 477: 466-70, 2011
²Walker et al., Science 326: 285-9, 2009.
³Huang et al., Nature 491: 406-12, 2012.

As noted above, passive transfer of various brNAbs individually or in combination has been shown to confer protection in macaque. Illustrative examples include the use of b12 (Burton et al., Proc Natl Acad Sci USA 108: 11181-6, 2011; Veazy et al., Nat Med 9: 343-6, 2003) 2G12 (Hessell et al., PLOS pathog 5: e1000433, 2009), PGT121 (Moldt et al., Proc Natl Acad Sci USA 109: 18921-5, 2012) and b12/2G12/2F5/4E10 combinations (Ruprecht et al., Vaccine. 21: 3370-3, 2003). It is also relevant to note that Ibalizumab (αCD4bs) monotherapy of HIV-1 infected individuals leads to a transient drop in viral load to nadir levels followed by a rebound in viral titre due to emergence of resistant mutants (Bruno & Jacobson, Antimicrob Chemother 65: 1839-41, 2010; Toma et al J Virol 85: 3872-80, 2011).

Studies show that these potent neutralizing antibodies, determined by Env glycoprotein epitopes, are critical in providing protection against viral challenge and it is hoped that further study of the epitopes in gp120/gp41 may provide better Env-based immunogens. However, it is unknown how to present these epitope in a format that will be any more effective in generating neutralizing antibodies than current vaccine Env-based immunogens. For example, three of the most broadly reactive neutralizing antibodies against HIV (2F5, 4E10 and Z13) bind to the membrane proximal ectodomain region (MPER) and contribute to protection yet the design of a vaccine which elicits antibodies with the same specificities has proven difficult.

Given the lack of effective therapies for the treatment or prevention of HIV infection there is an urgent need for immunogens and vectors capable of engendering immune responses effective in preventing or reducing HIV infection.

### SUMMARY

The present disclosure is predicated in part on the experimental and theoretical determination that forced evolution of an attenuated virus, having a mutation in the viral complex that mediates viral fusion and host cell entry, provides a process for producing second site suppressor mutants likely to serve as enhanced immunogens for the production of neutralizing antibodies targeting the complex. The effectiveness of this strategy is illustrated in Example 1 which describes a second site mutation in the conserved membrane proximal ectodomain region (MPER) of HIV-1 glycoprotein (gp) 41. The MPER mutant displays increased sensitivity of gp41 MPER dependent neutralizing antibodies. Example 2 provides a second site mutation in the external glycan shield region (comprising asparagine-linked oligosaccharides) of glycoprotein (gp) 120 variable region 1 (V1). The V1 glycosylation mutant displays increased sensitivity of gp120 glycan dependent neutralizing antibodies. Example 3 shows how these two mutants have been combined to produce an Env mutant displaying enhanced sensitivities against each of the broadly neutralizing antibodies 2G12, PGT121, PGT126 and 4E10 (see Table A and FIGS. 20 and 21). As a result, the present invention enables for the first time a rational process for producing a remodelled gp120-gp41 immunogen to engender or select neutralizing antibodies in a mammalian subject.

While described with respect to HIV, the present invention extends to any lentivirus envelope immunogen such as one in respect to FIV, SIV and BIV. Furthermore, the invention extends to forced evolution of virus envelope protein selected from the group comprising a Flavivirus (e.g. hepatitis C virus), Coronavirus, Herpesvirus, Hepadnavirus, Retrovirus (including HIV), Orthomyxovirus (e.g., influenza) or Paramyxovirus (e.g. measles virus) envelope proteins.

The HIV-1 envelope glycoprotein complex comprises a trimer of gp120 subunits in non-covalent association with a trimer of transmembrane gp41 subunits and mediates viral attachment membrane fusion and viral entry. A gp120-gp41 association site is formed by the terminal segments of C1 and C5 of gp120 and the central disulfide-bonded region of gp41 (see FIG. 1). Thus, in one aspect of the present invention, a process is provided for producing or selecting a modified immunogen to confer a neutralizing antibody response to a target antigen in a mammal. In some embodiments, the antibody response is enhanced over that conferred by the unmodified immunogen. In one embodiment relating to HIV Env, the process generally comprises: step (i) maintaining a cell to cell transmission attenuated HIV particle comprising a gp120-gp41 association site mutation which attenuates cell to cell transmission by the HIV particle as well as infection by cell-freeviral particles in a permissive cell for a time sufficient to promote the development of a transmission competent variant (TCV). In some embodiments, the process comprises step (ii) isolating a transmission competent variant (TCV) HIV particle from step (i). In another embodiment, the process comprises step (iii) determining the amino acid sequence of all or part of Env from the TCV in step (ii) to identify a second site suppressor mutation.

Reference to a “second site suppressor” mutation refers herein to Env comprising at least “a second mutation” wherein the second mutation affects the phenotype which is caused by a first pre-determined mutation at a distinct location to the second mutation. In accordance with the present invention, the first mutation is a mutation in the gp120-gp41 association site which attenuates the viral particle such that cell to cell transmission and cell free virus infectivity is disabled.

In some embodiments, the association site mutation forms a pseudoreversion or reversion mutant and this embodiment is also encompassed. In some embodiments, the first mutation is deleterious and the second mutation or the pseudoreversion mutation complements the phenotype of the first mutation. While the invention is described with respect to second site mutations, the skilled person will understand that the invention is not limited to a “second” site and that “third” or “fourth” etc site mutations may be employed.

In some embodiments, the process comprises assessing the sensitivity of the transmission competent variant (TCV) to a neutralizing antibody. As known to those of skill in the art neutralization assays may be achieved using a number of different protocols. While the entire TCV from (ii) may be tested, it will be apparent that any variant comprising the gp120-gp41 association site mutation or a reversion mutation and/or a second site suppressor mutation may be assayed. In some embodiments, the second site mutation may be transferred into an infectious or pseudotyped virus particle for assessment.

In some embodiments, the gp120-gp41 association site mutant comprises a mutation in the central disulfide bonded region (DSR) of gp41 as alignment of the DSR from a range of HIV types is set out in FIG. 9. The DSR contacts gp120 and facilitates infection. Alternative association site mutants comprise mutations in the N-terminal or C-terminal segments of gp120 such as the terminal segment of C1 and C5 of gp120 (Binley et al. 2000). In one embodiment, the DSR mutation is of the conserved K601 within the DSR (using the amino acid numbering based on HXB2 Env, see FIGS. 17 and 9). In one embodiment, the DSR mutation is of conserved W596 within the DSR (using the amino acid numbering based on HXB2 Env). In one embodiment, the DSR mutation is W596L and K601D within the DSR. Further substitutions at these residues (596 and 601) are contemplated, such as conservative substitutions. However, mutants are only selected which retain the cell-cell transmission attenuated phenotype. In some embodiments, the mutations result in shedding of gp120 from the glycoprotein complex and block infectivity. Illustrative mutations are represented diagrammatically in FIG. 17.

In some embodiments, the modified Env immunogen binds preferentially to neutralizing antibodies that recognise the V1/V2 region of gp120. In other embodiments, the modified Env immunogen binds preferentially to neutralizing antibodies that bind to the V3 region of gp120. In other embodiments, the modified Env immunogen binds preferentially to neutralizing antibodies that bind to the MPER region of gp41. In some embodiments, the modified Env immunogen binds preferentially to neutralizing antibodies that recognise the V1/V2 region of gp120 and the MPER region of gp41.

In one embodiment, the specification enables a modified HIV envelope glycoprotein (Env) antigen or a lipid containing vehicle comprising same wherein the Env antigen comprises one of: (i) a second site suppressor mutation in residue 674 of the membrane proximal ectodomain region (MPER) of HIV gp41; (ii) a second site suppressor mutation which ablates a glycosylation site in the variable region (V1) region of gp120; or (iii) a second site suppressor mutation ablating a glycosylation site in the V1 region of gp120 and a second site suppressor mutation in residue 674 of the MPER of HIV gp41.

In another aspect, the present invention provides a modified HIV Env immunogen wherein the Env immunogen comprises: (i) a gp120-gp41 association site mutation or a reversion or pseudoreversion mutation thereof and a second site suppressor mutation in MPER; (ii) a gp120-gp41 association site mutation or a reversion or pseudoreversion mutation thereof and a second site suppressor mutation in a glycosylation site in the V1 region; or (iii) a gp120-gp41 association site mutation or a reversion or pseudoreversion mutation thereof and a second site suppressor mutation in a glycosylation site in the V1 region and a second site suppressor mutation in MPER.

Reference herein “MPER” means the conserved 23-residue tryptophan-rich domain which connects the helical region 2 (HR2) of the gp41 ectodomain to the transmembrane domain.

In certain embodiments the Env antigen comprises a gp120-gp41 association site mutation or a reversion or pseudoreversion mutation thereof in the disulfide bonded region (DSR) of gp120, preferrably at K601 and/or W596.

Examples of the mutation at K601 and W596 include K601D, K601H, K601N, K601Q and K601R, and W596I, W596L, W596H, W596M, W596Y, W596F and W596A.

It is also preferred that the Env antigen comprises a mutation such that residue 674 is other than aspartic acid and is preferrably is glutamic acid.

Glycosylation site mutations in V1 of HIV gp120 are preferably ΔN139INN or T138N or a mutation of asparagine(s), threonine(s) or serine(s) in other HIV strains that ablate analogous glycosylation sites.

The lipid vehicle may be a human immunodeficiency virus like particle (HIVLP) or an enveloped virus or virus-like particle that is other than human immunodeficiency virus. Examples of relevant non HIV viruses include SIV, murine leukemia virus and other retroviruses, vesicular stomatitis virus, rabies virus, herpesvirus and hepadnavirus. Clearly the lipid vehicle may also be a non-viral lipid.

In another aspect the present invention provides a modified Env antigen comprises a mutation selected from the group consisting of ΔN139INN/W596L/K601H/D674E, ΔN139INN/W596L/K601D/D674E, ΔN139INN/W596L/K601N/D674E, W596L/K601H/D674E, ΔN139INN/W596L/K601H, T138N/W596L/K601H/D674E, T138N/ΔN139INN, T138N, ΔN139INN and a mutation of asparagine(s), threonine(s) or serine(s) in other HIV strains that ablate analogous glycosylation sites.

In yet another aspect the present invention provides an isolated nucleic acid molecule encoding the modified Env antigen of the present invention.

In a still further aspect the present invention provides a composition comprising the Env antigen or lipid vehicle of the present invention a pharmaceutically or physiologically acceptable carrier or diluent. The composition may also comprise other HIV antigens.

The present invention also provides a method of eliciting an immune response in a subject, the method comprising administering an effective amount of a composition according to the present invention for a time and under conditions sufficient to elicit an immune response. The immune response may comprise the production of neutralizing antibodies or the production of antibodies that prevent HIV replication through mechanisms other than neutralization.

In some embodiments, the modified or isolated Env immunogen is provided in a lipid containing vehicle such as a virus-like particle (VLP) or other lipid containing vehicle.

In some embodiments, the gp120-gp41 association site mutation is a DSR mutation. In an illustrative embodiment, the DSR mutation is in K601 such as K601D or a conservative substitution thereof (e.g. K601E). Exemplary substitutions are set out in Table 2. In some embodiments, the association site mutation reversion or pseudoreversion is K601K (reversion), K601H (pseudoreversion) or K601N (pseudoreversion) or a conservative substitution for lysine such as glutamine (K601Q) or arginine (K601R).

In some embodiments, the MPER mutation is D674E of HIV gp41. In one illustrative embodiment the Env immunogen comprises the MPER mutation D674E together with the DSR pseudoreversion K601H/N and the DSR mutation W596L.

In some embodiments, mutation in VI of HIV gp120 is a glycosylation site mutation.

In some embodiments, mutation in V1 of HIV gp120 is the glycosylation mutant, ΔN¹³⁹INN. This deletion ablates overlapping Asn¹⁴¹-Asn¹⁴²-Ser-Ser potential N-linked glycosylation sequons (PNGS) in V1. Corresponding mutations are made to conserved asparagine-rich portions of V1 of different strains. An alignment of the V1 regions from multiple strains is set out in FIG. 12A.

In other embodiments the mutation in V1 of HIV gp120 is the glycosylation mutant, T138N. This substitution ablates the Asn136 potential N-linked glycosylation sequons (PNGS) in V1.

In some embodiments, the present invention provides a nucleic acid molecule encoding a modified HIV Env immunogen of the present invention, plasmids, expression vectors and cells comprising same. In one illustrative embodiment, the nucleic acid molecule encodes an Env immunogen comprising W596L and K601H substitutions in the DSR of gp41, a substitution D674E in the MPER of gp41 and a deletion comprising ΔN¹³⁹INN of V1 of gp120. Corresponding glycosylation site mutations include mutation/ablation of one or two PNGS in V1, as highlighted in FIG. 12A. An illustrative embodiment, the Env immunogen comprises W596L, K601H, D674E.

The invention provides an Env immunogen or a nucleic acid molecule encoding same identified, produced or selected by the process described herein for identifying, producing or selecting modified Env immunogens.

In some embodiments, the present invention provides a composition comprising a remodelled Env immunogen or a vector encoding same as described herein and a pharmaceutically or physiologically acceptable carrier or diluent.

In some embodiments, the composition is for use in therapy, such as HIV prophylaxis or treatment.

Vaccine compositions are contemplated comprising a HIV Env immunogen in a lipid containing particle as an immunologically active component. Vaccine compositions (vaccines) may also contain additional components to enhance the immunological activity of the active component in a mammalian subject, such as an adjuvant.

In another embodiments, the present invention provides the composition in, or in the manufacture of a medicament for, the treatment or prevention of HIV infection.

Also contemplated are kits or solid substrates comprising a remodelled Env immunogen as described herein or a lipid containing complex comprising same.

The above summary is not and should not be seen in any way as an exhaustive recitation of all embodiments of the present invention.

### BRIEF DESCRIPTION OF THE FIGURES

If figures contain colour representations or entities, coloured versions of the figures are available from the Patentee upon request or from an appropriate Patent Office. A fee may be imposed if obtained from a Patent Office.

FIG. 1 provides representations illustrating the location and phenotype and WL/KD in the context of the gp120-gp41 Ectodomain. gp120 was drawn using the coordinates 3JWD (Pancera et al, 2010) and 2QAD (Huang et al, 2007). The gp120 core is coloured blue, CD4 binding site (CD4bs) and CCR5-binding site (CCR5bs) in cyan and magenta, respectively, gp41 binding site in green. The DSR (green) and MPER were drawn using the coordinates 1IM7 (Du et al, 2002) and 2PV6 (Sun et al, 2008). The N- and C-terminal helical segments of the MPER are colored purple and magenta respectively and the interhelical hinge in yellow. The sidechains of the aromatic/hydrophobic face of the MPER that inserts into the lipid phase of the membrane are indicated. FP: fusion peptide. (B) gp120-gp41 association. Lysates of metabolically labelled WT, K601D, WL/KD, W596L or empty vector (mock) transfected 293T cells (c) and corresponding culture supernatants (s) were immuno precipitated with pooled IgG from HIV-1-infected persons and protein G-Sepharose. Proteins were analysed by reducing SDS-PAGE and phosphor imaging. (C) Cell-cell fusion activity. 293T effector cells were cotransfected with pCAG-T7 plus pcDNA3.1-AD8 env plasmids and then cocultured (18 h, 37° C.) with CD4 plus CCR5-expressing BHK21 harbouring a luciferase reporter plasmid. The mean relative light units (RLU) of a representative experiment are shown. (D) 14-day replication kinetics in U87.CD4.CCR5 cells. Virus produced in 293T cells was normalised for RT activity and used to infect U87.CD4.CCR5 cells. The RT activity of the culture supernatant was measured at days 3, 7, 10 and 14, post infection. The mean RT activity±standard deviation of triplicate samples is shown.

FIG. 2 illustrates the results of long-term culture of W596L/K601D virus. (A) Wild type and W596L/K601D-mutated HIV-1_{AD8} virus stocks produced by transfected 293T cells were normalized according to RT activity and used to infect U87.CD4.CCR5 cells. The cell-free virus obtained at day 10 was filtered through a 0.45 μm nitrocellulose filter, normalised according to RT activity and used to infect fresh U87.CD4.CCR5 cells. Viruses were subjected to 5 sequential passages in total. (B) Infection of U87.CD4.CCR5 cells was initiated with VSV G-pseudotyped WT and W596L/K601D mutant viruses. The cells were trypsinized 24-h later to remove residually adsorbed viruses. The passaging procedure described in A was then followed. The results shown represent the mean RT activity±standard deviation of triplicate samples. (C) Reversion pathways in WLKD-1 and WLKD-2. The env region was PCR amplified from proviral DNA isolated at days 10, 20, 30, 40 and 50, cloned into pΔKAD8 env and sequenced. Upper case lettering connected by a bold horizontal line denotes a major evolutionary pathway, while lower case lettering connected viathin horizontal lines denotes a minor pathway. Lower case lettering only: low-frequency genotypes arising at the specified days.

FIG. 3 provides a graphical representation of replication of representative WLKD-1 (A) and WLKD-2 (B) clones in U87.CD4.CCR5 cells. Virus produced in 293T cells was normalised according to RT activity and used to infect U87.CD4.CCR5 cells. Reverse transcriptase activity in culture supernatants was measured at days 3, 7, 10 and 14. The mean RT activity±standard deviation of triplicate samples is shown. (C) Single-cycle infectivity was determined in U87.CD4.CCR5 infected with env-pseudotyped luciferase reporter viruses at 48-h post-infection. Luciferase activity was normalised against the RT activity present in each virus inoculum. The mean RLU±standard errors of 3 independent assays are presented here. **, P<0.01, unpaired t test assuming unequal variances. (D) Serial 10-fold dilutions of env-pseudotyped luciferase reporter viruses were added to U87.CD4.CCR5 target cells and luciferase activity determined 48-h later. The mean RLU±standard deviations of a representative experiment are presented.

FIG. 4 provides graphical representation of the spread of cell-associated virus. U87.CD4.CCR5 cells were inoculated with VSV G-pseudotyped HIV-1 particles [50,000 (A) or 20,000 (B) cpm of RT activity per inoculum] and then trypsinized 24-h later to remove residual adsorbed virus. The cells were then replated and cultured for a further 10 days. The results shown represent the mean RT activity±standard deviation of triplicate samples. (C) As for B except that 1 μM C34 peptide was maintained in the culture following the trypsinization step.

FIG. 5 is a representation of data illustrating subunit association. (A) Western blotting of selected revertant clones. At 48-h post-transfection, pΔKAD env-transfected 293T cells were lysed and subjected to reducing SDS-PAGE followed by western blotting with DV012 to gp120 (upper panel) and mAb C8 to gp41 (lower panel). (B) gp120-gp41 association was determined as for FIG. 1B. gp120-shedding index was calculated according to the formula: ([mutant gp120]ₛᵤₚₑᵣₙₐₜₐₙₜ×[WT gp120]_{cell})/([mutant gp120]_{cell}×[WT gp120]ₛᵤₚₑᵣₙₐₜₐₙₜ) (Helseth et al, 1991). (C) Characterization of virions produced by pAD8 infectious clones. Pelleted HIV-1 virions were analysed by Western blotting using DV-012 (upper panel) and pooled IgG from HIV-1-infected persons (lower panel).gp120 and p24 band intensities were determined using a Licor Odyssey scanner.

FIG. 6 is a graphical representation of cell-cell fusion activities of revertant Envs. 293T effectors (co-transfected with pcDNA3.1AD8env plus pCAG-T7) were co-cultured with BHK-21 targets co-transfected with pT4luc and pcCCR5 vectors for 18 h and then lysed and assayed for luciferase activity. The data presented here are the means±standard errors of 3 independent assays. *, P<0.05, unpaired t test assuming unequal variances.

FIG. 7 is a graphical representation showing the sensitivity of revertants to neutralizing agents. The TZM-b1 cells were incubated with virus-inhibitor complexes for 2 days prior to lysis and then assayed for luciferase activity. For the maraviroc experiment, target cells were incubated in the presence of the inhibitor for 1-h prior to inoculation. Neutralizing activities are reported as the average percent maximal luciferase activity. The data presented here are the means±standard errors; n=2 for IgGb12, 2F5 and 4E10; n=3 for C34 and maraviroc.

FIG. 8 is a pictorial representation of modeling amino acid changes at position 674 modeled on the dodecylphosphocholine-associated MPER peptide (PDB entry 2PV6). The Asp-674 (A), Glu-674 (B), and Asn-674 (C) models were produced with Swiss Model and drawn with Pymol. The N- and C-terminal helical segments are shown in purple and magenta respectively, while Phe-673 that forms part of the hinge region is in yellow. The aromatic layer and Ile-675, which are associated with the hydrophobic phase of the lipid are indicated.

FIG. 9 is a representation illustrating the location and phenotype of K601D. A, Linear map of gp41. fp: fusion peptide, HR1: helical region 1, DSR: disulfide bonded region, HR2: helical region 2, TMD: transmembrane domain, CT: cytoplasmic tail. Residue numbering is in accordance with HXB2 Env. Magenta circles denote the residues mutated in this study. B, Alignment of the AD8 DSR amino acid sequence with consensus (CONS) sequences derived from HIV-1 main-group subtypes and circulating recombinant forms (Los Alamos National Laboratory's HIV sequence database, operated by Los Alamos National Security, LLC, for the U.S. Department of Energy's National Nuclear Security Administration). The residue numbers of amino acids implicated in gp120 association[27,75] are indicated. C, gp120-gp41 association. Lysates of metabolically labelled WT, K601D or empty vector (mock) transfected 293T cells (c) and corresponding culture supernatants (s) were immunoprecipitated with IgG14 and protein G Sepharose. Proteins were analysed under reducing conditions in SDS-PAGE and scanned in a Fuji phosphorimager. Panel prepared from a single gel using Adobe Photoshop. D, Virion characterization. HIV-1 virions produced by transfected 293T cells were pelleted from the culture supernatant through a 25% sucrose cushion and analysed by reducing SDS-PAGE and Western blotting with DV-012 (anti-gp120) and mAb 183 (anti-CA). K601D.1 and K601D.2 are 2 independent clones of pAD8-K601D; NL4.3: virus derived from the pNL4.3 clone [109]. E, Lysates of 293T cells transfected with 1 or 0.25 μg pcDNA3.1-AD8env vectors analysed by Western blot using the gp41-specific mAb, C8. The asterisk denotes degradation products of gp160. F, Cell-cell fusion. 293T effector cells were cotransfected with 1 μg pCAG-T7 plus 1 or 0.25 μg of pcDNA3.1-AD8env and then cocultured (16 h, 37° C.) with BHK21 target cells that had been cotransfected with pc.CCR5 and pT4luc. The mean relative light units (RLU) standard deviation of a representative experiment is shown. G, Long-term PBMC culture of HIV-1_{AD8}-WT and HIV-1_{AD8}-K601D. Viruses produced by pAD8-transfected 293T cells were normalized according to RT activity and then used to infect independent cultures of PHA stimulated PBMCs (cultures P0 and P2). The PBMCs used in each passage were obtained from different donors. Cell-free virus collected at day 10 of each passage was normalized for RT activity and used to infect fresh PHA stimulated PBMCs. The mean RT activity±standard deviation of duplicate samples obtained from culture supernatants is shown.

FIG. 10 is a representation illustrating the genotypes of HIV-1_{AD8}-K601D revertants. The env region of proviral DNA isolated at days 20, 30, 40 and 50 from P0 (A), and days 30 and 50 from P2 (B) was amplified by PCR and cloned into pGEM-T or pΔKAD8env. The entire env region present in individual clones was sequenced using Bigdye terminator 3.1. The amino acid numbering is based on HXB2 Env.

FIG. 11 is a representation illustrating the analysis of revertants. A and B, 14-day replication kinetics of representative P0 and P2 genotypes in PBMCs. Virus produced in 293T cells was normalised according to RT activity and used to infect PHA stimulated PBMCs from 2 independent donors (A and B, respectively). RT activity was measured in culture supernatants obtained at days 3, 7, 10 and 14 postinfection. The mean RT activity±standard deviation of duplicate samples is shown. C, gp120-gp41 association. Lysates of metabolically labelled Env-expressing cells (c) and corresponding culture supernatants (s) were immunoprecipitated with IgG14 plus protein G-Sepharose and subjected to reducing SDS-PAGE and phosphorimager scanning. The panel was prepared from 2 gels obtained from a single experiment (representative of 3 independent experiments) using Adobe Photoshop. gp120-shedding index was calculated according to the formula: ([mutant gp120]ₛᵤₚₑᵣₙₐₜₐₙₜ×[WT gp120]_{cell})/([mutant gp120]_{cell}×[WT gp120]ₛᵤₚₑᵣₙₐₜₐₙₜ) [26]. The data shown are the mean association indices±standard error from at least 3 independent experiments. D, Cell-cell fusion activities of representative P0 and P2 genotypes. Assays were conducted with 0.25 μg AD8 Env expression plasmids as for FIG. 9E. Mean RLU±standard error is shown (n>3). ***, P<0.001 versus K601D, 2-tailed unpaired t test assuming unequal variances. E,T138N and ΔN¹³⁹INN mutations on a WT Env background do not affect cell-cell fusion activity (Mean RLU±standard error, n>3). F, T138N and ΔN¹³⁹INN mutations on a WT Env background do not affect the ability of Env-pseudotyped luciferase reporter viruses to mediate a single cycle of infection in U87.CD4.CCR5 cells. Mean RLU±standard deviation from a representative experiment is shown.

FIG. 12 is a representation illustrating the evidence for a specific functional linkage between position 601 of the DSR and the Asn¹³⁶ and Asn¹⁴² glycans of V1. A, Alignment of V1V2 and corresponding DSR sequences. PNGSs are highlighted in green and numbered according to HXB2 Env. Variable residues in the DSR are highlighted in grey. CONS, subtype consensus sequence. B, Cell-cell fusion. Assays were conducted with 0.25 μg Env expression plasmids as for FIG. 9F. Mean RLU±standard error (n≧4) is shown. |, P<0.05; ∥, P<0.01 versus WT; *, P<0.05; ** P<0.01 versus K601N; 2-tailed unpaired t test assuming unequal variances. C, Lysates of Env-expressing 293T cells were analysed by Western blot using the gp41-specific mAb, C8. The asterisk denotes degradation products of gp160. D, gp120-gp41 association. Lysates of metabolically labelled Env-expressing cells (c) and corresponding culture supernatants (s) were immunoprecipitated with HIVIG plus protein G-Sepharose and subjected to reducing SDS-PAGE and phosphorimager scanning. The data are representative of 2 independent experiments. gp120-shedding indices (mean±standard error) are shown below the corresponding immunoprecipitations and were calculated as for FIG. 11C from 2 independent experiments.

FIG. 13 is a representation illustrating the receptor binding properties and C34 susceptibility of revertants. A, CD4 binding by WT and mutated gp120 molecules. Soluble CD4 binding curves were obtained by incubating a constant amount of biosynthetically labelled WT and mutated gp120 with the indicated amounts of sCD4. gp120-sCD4 complexes were coimmunoprecipitated with mAb OKT4 and protein G-Sepharose, followed by reducing SDS-PAGE and densitometry of gp120 bands. gp120-sCD4 binding is expressed as a percentage of gp120 immunoprecipitated by IgG14. The mean±standard deviation from 2 independent experiments is shown. B, Inhibition of cell-cell fusion by sCD4. Assays were conducted with 0.25 μg Env expression plasmids as for FIG. 9F except that Env-293T cells were incubated with a dilution series of sCD4 for 3.5 h prior to coculture with CD4-plus-CCR5-expressing BHK21 targets for 8 h. The data are expressed as a percentage of the maximal fusion achieved by each Env and represent the means±standard error from 4 independent experiments. C, Utilization of CCR5 mutants in cell-cell fusion by T138N/L494I/K601N and ΔN¹³⁹INN/K601N. Assays were conducted with 0.25 μg Env expression plasmids as for FIG. 9F. The fusion activity of each Env clone was normalized against its fusion activity with WT CCR5. Mean relative fusion activity±standard deviation is shown from a representative experiment. CCR5 N-terminal domain: Nt, extracellular loops 1, 2, and 3: ECL1, ECL2 and ECL3, respectively. D, Utilization of the CCR5-Y14N coreceptor mutant in cell-cell fusion by T138N/L494I/K601N and ΔN¹³⁹INN/K601N. 293T effector cells cotransfected with 1 μg pCAG-T7 plus 0.25 μg Env expression plasmids were cocultured (16 h, 37° C.) with BHK21 targets cotransfected with 1 pT4luc plus the indicated amounts of WT or Y14N-mutated pc.CCR5 prior to luciferase assay. The fusion activity of each Env clone was normalized against its fusion activity with WT CCR5. Mean relative fusion activity±standard deviation is shown from a representative experiment. E, Sensitivity of T138N/L494I/K601N and ΔN¹³⁹INN/K601N to the fusion inhibitor peptide, C34. 293T effector cells cotransfected with 1 μg pCAG-T7 plus 0.25 μg Env expression plasmids were cocultured (16 h, 37° C.) with BHK21 targets cotransfected with pT4luc plus pc.CCR5 in the presence of the indicated amounts of C34 prior to luciferase assay. The data are expressed as a percentage of cell-cell fusion activity in the absence of inhibitor (mean±standard error; n=3).

FIG. 14 is a representation illustrating the effects of T138N, ΔN¹³⁹INN and L4941 mutations on gp120-gp41 association and cell-cell fusion activities of DSR mutants. A, gp120-gp41 association. Lysates of metabolically labelled Env-expressing cells (c) and corresponding culture supernatants (s) were immunoprecipitated with IgG14 plus protein G-Sepharose and subjected to SDS-PAGE and phosphorimager scanning. The panel was prepared from 3 gels obtained from a single experiment using Adobe Photoshop. gp120-shedding indices (mean±standard error) are shown below the corresponding imunoprecipitations and were calculated as for FIG. 11C from at least 3 independent experiments. B, Cell-cell fusion. Assays were conducted with 0.25 μg Env expression plasmids as for FIG. 9F. Mean RLU±standard error (n≧3) is shown. *, P<0.05; ** P<0.01; **, P<0.001; 2-tailed unpaired t test assuming unequal variances.

FIG. 15 is graphical representations illustrating the sensitivity of T138N and ΔN¹³⁹INN mutant pseudovirions to NAbs. U87.CD4.CCR5 cells were incubated with pseudovirus-IgG mixtures for 2 days prior to lysis and assay for luciferase activity. Neutralizing activities were measured in triplicate and reported as the average percent luciferase activity. The data are representative of 2-4 independent experiments. WT: blue squares, T138N: red triangles, ΔN¹³⁹INN: green “X”s.

FIG. 16 is a representation illustrating structural models. A, Homology model of oligomannose-glycosylated AD8 V1V2 based on the crystal structure of CAP45 V1V2 [7]. The V1V2 model, generated using the Modeller algorithm [106] within Discovery Studio 3.0, was glycosylated in silico with oligomannose side chains using the glycosciences.de server [107,108]. The β2-β3 hairpin that forms the V1V2 base is colored green, V1 in yellow, V2 in orange. PNGSs (Asn residues shown in CPK) and oligomannose side chains are colored according to the V1V2 subdomain to which they are attached. B, Model of oligomannose-glycosylated gp120 monomer. The model, prepared with the UCSF Chimera package [110], is based on the crystal structures of the complex formed between HXBc2 gp120 with gp41-interactive region (gp120 residues 31-284, 334-501), sCD4 and 48 d Fab (PDB ID 3JWD) and the YU2 gp120 (residues 285-333)-418d Fab-sCD4 complex (PDB ID 2QAD) [5,8]. Oligomannose addition was performed in silico as for A. The gp41 association site formed by the N- and C-terminal segments is colored green, the 7 stranded β-sandwich in purple, layer 1 and the V1V2 β2-β3 hairpin base in green, layer 2 in pink, the outer domain in red. Asn residues representing PNGSs are shown in CPK. Oligomannose glycans implicated in 2G12 recognition are colored crimson. The homology models were drawn using Pymol. C, Alignment of V1V2 amino acid sequences. Selected V1V2 sequences were initially aligned using clustalx and then adjusted manually. PNGSs are highlighted in green. Residue numbering is according to HXB2. sens: neutralization-sensitive; res: neutralization-resistant. ^{a}, Neutralization susceptibility of Envs derived from subtype B HIV-1 reference strains as determined using macaque antisera raised to SF162 gp140 immunogen[36]; ^{b}, Neutralization susceptibility phenotype associated with primary (M1) and chronic (M47, M46, M32 and M31) phase subtype A V1V2 sequences as determined with autologous plasma [42]; ^{c} Consensus V1V2 sequences derived from neutralization-sensitive (VP-1CON) and neutralization-resistant (VP-2CON) isolates obtained from a patient with cross-reactive neutralizing activity as determined with autologous plasma [44].

FIG. 17 is a pictorial representation of the mutations identified herein.

FIG. 18 is a representation illustrating the location of the ΔN¹³⁹INN/W596/K601/D674 (“WL/KH/DE” mutation) mutations in the context of Env.

FIG. 19 is a representation illustrating the infectivity of ΔNINN/WL/KH/DE Env-pseudotyped luciferase reporter viruses for U87.CD4.CCR5 cells. Serial 10-fold dilutions of WT or ΔNINN/WL/KH/DE Env-pseudotyped luciferase reporter viruses were added to U87.CD4.CCR5 target cells and luciferase activity determined 48-h later. The mean RLU standard deviations of a representative experiment are presented. Empty: luciferase reporter viruses lacking Env.

FIG. 20 is a table setting out the neutralization properties and epitopes of brNAbs.

FIG. 21 is graphical representations illustrating the sensitivity of ΔNINN/WL/KH/DE Env-pseudotyped luciferase reporter viruses to brNAbs. U87.CD4.CCR5 cells were incubated with pseudovirus-IgG mixtures for 2 days prior to lysis and assay for luciferase activity. Neutralizing activities were measured in triplicate and reported as the average percent luciferase activity.

FIG. 22 is graphical representations illustrating the sensitivity of ΔNINN/WL/KH/DE Env-pseudotyped luciferase reporter viruses to brNAbs PGT121, PGT126, IgGb12, VRCO1 and 10E8. Control IgG1 is IgG1 obtained from an individual who has not been exposed to HIV-1. U87.CD4.CCR5 cells were incubated with pseudovirus-IgG mixtures for 2 days prior to lysis and assay for luciferase activity. The mean RLUs±standard errors of the means from 3 independent experiments are reported, except for control IgG1 (n=2).

FIG. 23 is a representation illustrating the incorporation of WT and ΔNINN/WL/KH/DE Env glycoproteins into HIVLPs produced by co-transfecting 293T cells with pcGagPolVpu plus pΔKAD8-WT (WT lane), or pcGagPolVpu plus pΔKAD8-ΔNINN/WL/KH/DE (ΔNINN/WL/KH/DE lane) using the Fugene HD procedure. Control HIVLPs lacking Env were produced by cotransfecting pcGagPolVpu with pCMV-Rev (No Env lane) into 293T cells. At 72 h post-transfection, HIVLPs present in culture supernatants were partially purified by ultracentrifugation through a 1.5 ml 25% sucrose cushion. The pelleted virions were resuspended in PBS and then subjected to SDS-PAGE under reducing conditions followed by Western blotting with a sheep polyclonal antiserum raised to recombinant gp120 (DV-012) and IgG purified from the plasma of a HIV-1-infected individual (HIV+IgG). Left-hand side panel: Western blot with DV-012 showing that the HIVLPs contain gp120 as well as the uncleaved Env precursor, gp160. Right-hand side panel: Blotting with HIV+IgG reveals Gag- and GagPol-derived products in addition to gp120 and gp160. M, molecular weight markers.

FIG. 24 is graphical representations illustrating the binding of brNAb PGT121 to ΔNINN/WL/KH/DE Env-containing HIVLPs in ELISA. 96-well ELISA plates were coated with HIVLP suspensions that had been adjusted to normalize the CA content (based on the intensity of the CA band observed in the western blot) at 37° C. for 2 h. The plates were blocked with 3% bovine serum albumin-PBS (37° C., 1 h). Left-hand side panel: a dilution series of PGT121 was added to the HIVLP-coated plates (1 h, 37° C. incubation) followed by horseradish peroxidase-conjugated rabbit immunoglobulins to human immunoglobulins (1 h, room temperature incubation). The assay was developed with 3,3′,5,5′-tetramethylbenzidine and the reaction terminated with 1N HCl. The background absorbance at 620 nm was subtracted from the absorbance at 450 nm. The ELISA was conducted in the absence of detergent. The data are the mean absorbance values±standard error of the mean from 3 independent experiments. Right-hand side panel: The plate-bound HIVLPs were treated with 1% Triton X100 at 4° C. for 1 h prior to washing and addition of a mAb 183 (anti-CA) dilution series (1 h, 37° C. incubation) and then horseradish peroxidase conjugated rabbit immunoglobulins to mouse immunoglobulins. The ELISA was developed as described above. O.D.: optical density at 450 nm minus optical density at 620 nm.

### BRIEF DESCRIPTION OF THE TABLES

Table A illustrates available neutralizing antibodies, their specificity in Env and their neutralizing potency and breadth.

Table 1 provides an amino acid sub-classification.

Table 2 provides exemplary conservative amino acid substitutions.

### DETAILED DESCRIPTION

The subject invention is not limited to particular screening procedures for agents, specific formulations of agents and various medical methodologies, as such may vary.

Unless defined otherwise, all technical and scientific terms used herein have the same meanings as commonly understood by one of ordinary skill in the art to which this invention belongs. Any materials and methods similar or equivalent to those described herein can be used to practice or test the present invention. Practitioners are particularly directed to Sambrook et al., 1989 Chapters 16 and 17, Coligan et al., Current Protocols In Protein Science, John Wiley & Sons, Inc., 1995-1997, in particular Chapters 1, 5 and 6. and Ausubel et al., Current Protocols in Molecular Biology, Supplement 47, John Wiley & Sons, New York, 1999; Colowick and Kaplan, eds., Methods In Enzymology, Academic Press, Inc.; Weir and Blackwell, eds., Handbook of Experimental Immunology, Vols. I-IV, Blackwell Scientific Publications, 1986; Joklik ed., Virology, 3rd Edition, 1988; Fields and Knipe, eds, Fundamental Virology, 2nd Edition, 1991; Fields et al., eds, Virology, 3rd Edition, Lippincott-Raven, Philadelphia, Pa., 1996, for definitions and terms of the art and other methods known to the person skilled in the art.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the singular forms “a”, “an” and “the” include plural aspects unless the context clearly dictates otherwise.

Each embodiment in this specification is to be applied mutatis mutandis to every other embodiment unless expressly stated otherwise.

Thus, in one aspect of the present invention, a process is provided for producing a modified or isolated immunogen to confer a neutralizing antibody response to a target antigen in a mammalian subject which is enhanced over that conferred by the unmodified immunogen. In one embodiment relating to HIV Env, the process generally comprises the following steps: (i) maintaining a cell to cell transmission attenuated HIV particle comprising a gp120-gp41 association site mutation which attenuates cell to cell transmission in a permissive cell for a time sufficient to promote the development of a cell to cell transmission competent variant (TCV). In some embodiments, the process comprises (ii) isolating a cell to cell transmission replication competent variant (TCV) HIV particle from (i). In another embodiment, the process comprises (iii) determining the amino acid sequence of all or part of Env from the TCV in step (ii) to identify a second site suppressor mutation.

Reference to a “second site suppressor” includes a second mutation distant from a first mutation wherein the second mutation affects the phenotype which is caused by the first mutation. In some embodiments, the first mutation is deleterious and the second mutation complements the phenotype of the first mutation.

Reference to “enhanced” includes qualitative as well as quantitative improvement relative to a control (unmodified) immunogen. This may be determined using any method in the art such as by determining the epitope specificity, cross-clade sensitivity, IC50 or IC90s or percentage neutralisation by monoclonal or polyclonal antibodies (such as sera from an immunised subject). In some embodiments, neutralisation potency, breadth or sensitivity is improved by at least 20%, 40%, 60%, 90%, 100%, 200%, 300%, 400% etc. Qualitative differences can be determined readily such as by comparing the absence of neutralizing antibody to a particular epitope, moiety or region of Env compared to the presence of neutralizing antibody to that epitope, moiety or domain.

Reference to maintaining includes passage and serial passage of replication defective virus in a permissive cell. Suitable cells or cell lines are known in the art and include PMBC and U87 cells comprising CD4 and an appropriate viral co-receptor.

In some embodiments, the process comprises assessing the sensitivity of the transmission competent variant (TCV) to a neutralizing antibody. As known to those of skill in the art neutralization assays may be achieved using a number of different protocols. While the entire TCV from (ii) may be tested, it will be apparent that any TCV comprising the gp120-gp41 association site mutation and the second site suppressor mutation may be assayed. In some embodiments, the second site mutation may be transferred into another infectious particle for testing. In this way the mutations may be assessed against a background of at least two and preferably multiple clades and tiers of virus.

Neutralization assays are generally conducted using a panel of neutralizing agents such as antibodies as described in the examples. Exemplary monoclonal antibodies are PG16, 2G12, b12, 2F5, 4E10, VRC01, PGT121/8, PGT145 and 10E8.

Neutralization sensitivity assays are generally conducted to determine the ability of the modified Env in the context of a virion or lipid-containing vehicle to be neutralized by any at least two neutralizing antibodies recognising a combination of specificities (e.g., CD4bs and gp41MPER), (ii) by neutralizing antibodies that neutralise tier 1 and tier 2 isolates (ii) by neutralising antibodies that neutralise one or more HIV clades of interest; and to the potency of the neutralization with neutralizing antibodies (i.e. comparing the IC50 or IC90 values with control HIV particles selected from neutralisation sensitive or neutralization resistance particles known in the art.

Immunisation protocols are known to the skilled address and described herein. These may include the use of a range of adjuvants, sustained release formulations and administration protocols designed to test the ability of the remodelled Env immunogen to elicit an immune response, and preferably a neutralizing antibody response. Cross-clade neutralization assays are also known in the art and are contemplated herein.

In some embodiments, gp120-gp41 association site mutant comprises a mutation in the disulfide bonded region (DSR) of gp41. Alternative association site mutants may comprise mutations in the N-terminal or C-terminal segments of gp120. In one embodiment, the DSR mutation is of K601 within the DSR. In some embodiments, the K601 mutation is K601D.

In some embodiments, the process is conducted in vitro.

In some embodiments the virus is HIV, preferably HIV-1.

In some embodiments, the neutralizing antibody is a gp120 glycan directed antibody. The epitope recognised by a particular antibody is determined using standard art recognised protocols.

In other embodiments, the neutralizing antibody is a gp41 MPER directed antibody.

In further embodiments, the neutralizing antibody is a CD4 binding site directed antibody.

In a further embodiment, the process comprises assessing the sensitivity of the TCV or a virus comprising second site suppressor mutation to human sera or antibodies selected therefrom, or another potential anti-HIV agent, such as a fusion inhibitor peptide.

In some embodiments, the part of Env is the V1 domain of HIV gp120.

In some embodiments, the part of Env is the MPER domain of HIV gp41.

In some embodiments, a modified HIV envelope glycoprotein (Env) antigen or a lipid containing vehicle comprising same wherein the Env antigen comprises one of: (i) a second site suppressor mutation in residue 674 of the membrane proximal ectodomain region (MPER) of HIV gp41; (ii) a second site suppressor mutation which ablates a glycosylation site in the variable region (V1) region of gp120; or (iii) a second site suppressor mutation ablating a glycosylation site in the V1 region of gp120 and a second site suppressor mutation in residue 674 of the MPER of HIV gp41.

In another aspect, the present invention provides a modified HIV Env immunogen wherein the Env immunogen comprises: (i) a gp120-gp41 association site mutation or a reversion or pseudoreversion mutation thereof and/or a second site suppressor mutation in MPER; (ii) a gp120-gp41 association site mutation or a reversion or pseudoreversion mutation thereof and/or a second site suppressor mutation in a glycosylation site in the V1 region; or (iii) a gp120-gp41 association site or a reversion or pseudoreversion mutation thereof mutation and/or a second site suppressor mutation in a glycosylation site in the V1 region and a second site suppressor mutation in MPER.

Reference to Env immunogen or Env or Env polypeptide and the like includes an Env glycoprotein from any genotype, group, clade or isolate of HIV. The term further includes non-naturally occurring variants including portions of the full length Env provided they are able to display the portions of the glycoprotein necessary to induce a neutralizing immune response in a subject.

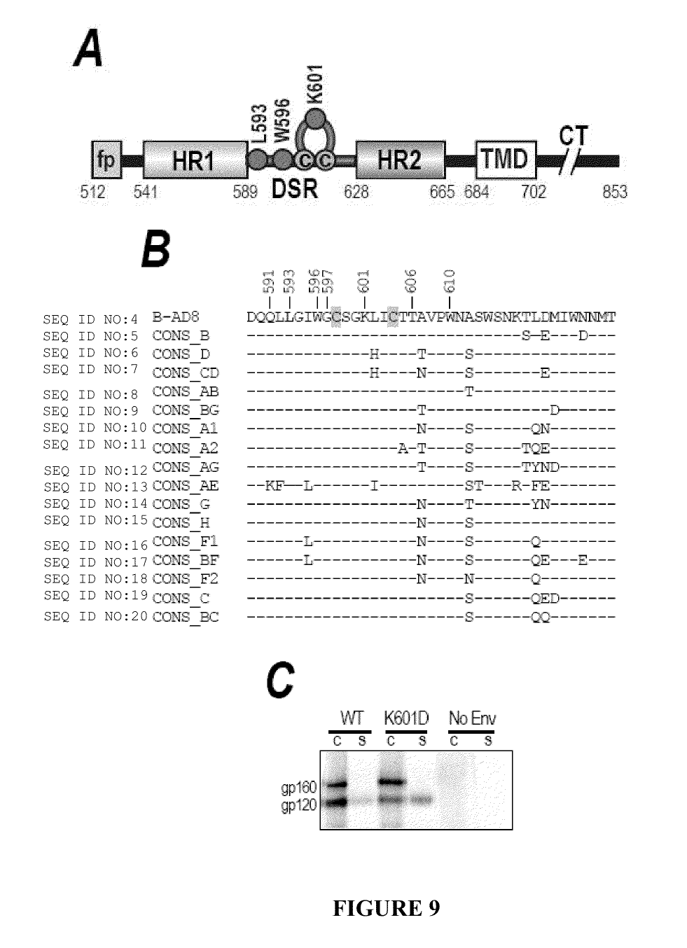

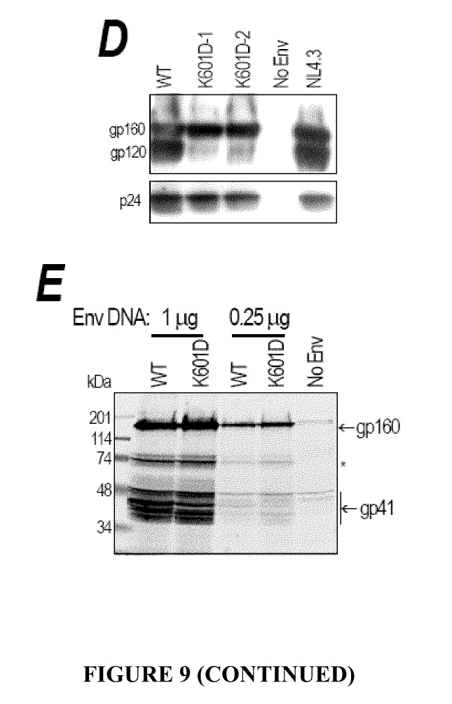

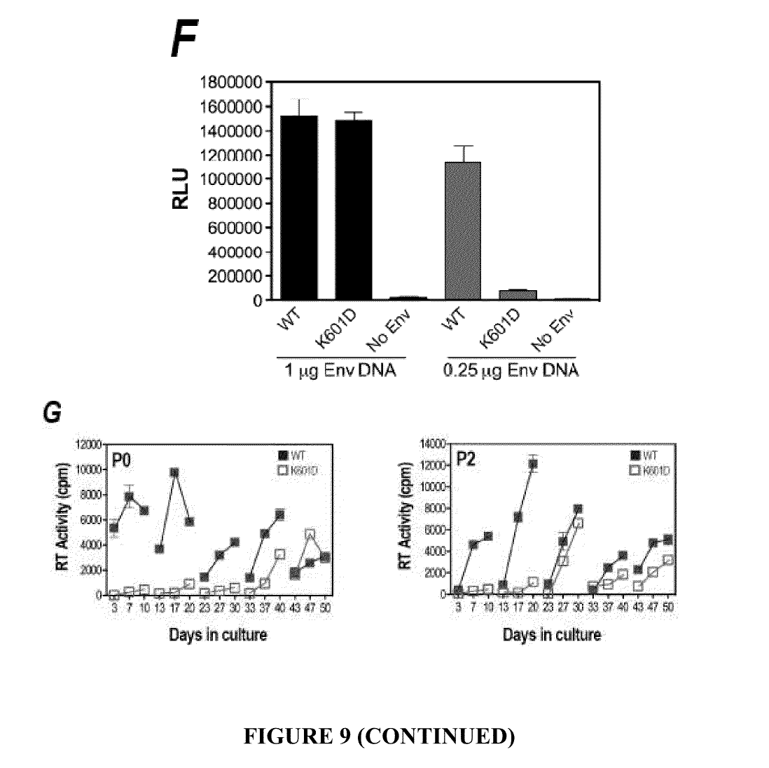

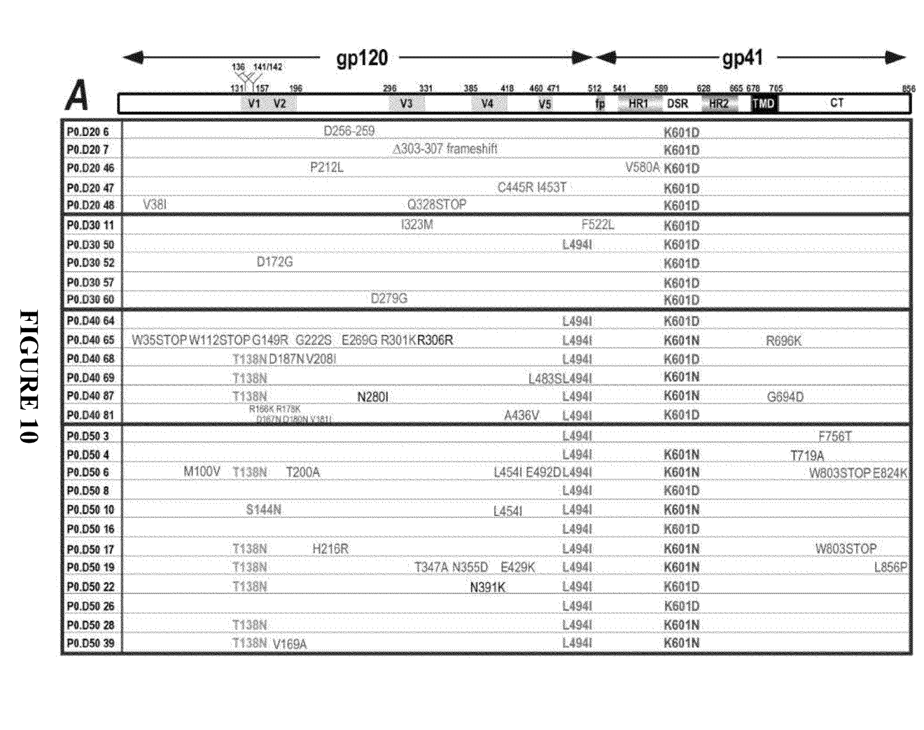

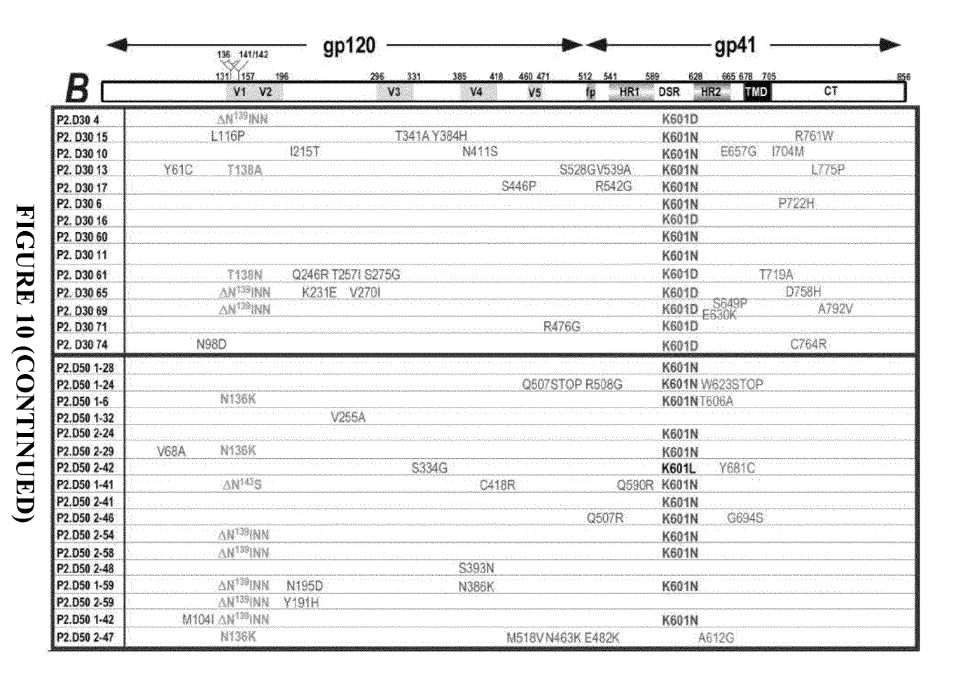

In nature, the Env complex comprises a trimer of gp120 subunits in non-covalent association with a trimer of transmembrane gp41 subunits and this complex mediates viral attachment, membrane fusion and viral entry (for review see 1 in Bibliography 2). Within gp120, five conserved regions (C1-C5) alternate with five variable regions (V1-V5). The conserved regions largely form the gp120 core comprising inner and outer subdomains that are bridged by four antiparallel β-strands (the bridging sheet), whereas the variable regions form external solvent-exposed loops (see 3, 4, 5, 6, 7, 8 in Bibliography 2). gp120 is anchored to the viral envelope by the trimeric transmembrane/fusion glycoprotein, gp41. The ectodomain of gp41 comprises an N-terminal fusion peptide linked through N- and C-terminal α-helical heptad repeat sequences (HR1 and HR2, respectively) to a C-terminal membrane anchor and cytoplasmic tail. A central disulfide-bonded loop region or DSR joins HR1 to HR2 (See FIG. 9A, B).

The membrane fusion and viral entry function of gp120-gp41 involves conformational changes that are triggered by receptors. CD4 ligation is believed to reorganize V1V2 and V3 to expose a binding site for the chemokine receptors CCR5 and CXCR4, which function as fusion cofactors (see 3, 4, 5, 6, 9, 10, 11, 12 in Bibliography 2). The V3 loop mediates important contacts with the negatively charged N-terminal domain and extracellular loop 2 of CCR5 and CXCR4 and determines the chemokine receptor preference of HIV-1 isolates. In a virion context, CD4 binding causes an “opening up” of the gp120 trimer due to outward rotation and displacement of gp120 monomers (see 10, 12 in Bibliography 2).

The gp120-receptor interactions cause gp41 to transition from a dormant metastable structure into a fusion active state (see 1, 2, 13, 14 in Bibliography 2). Structural transitions in gp41 that are associated with fusion function include the insertion of the fusion peptide into the target membrane and formation of a “prehairpin intermediate” structure wherein a triple-stranded coiled coil of HR1 segments provides a binding surface for the HR2 (see 15, 16, 17, 18, 10 in Bibliography 2). Antiparallel HR1-HR2 interactions forma 6-helix bundle which opposes the N- and C-terminal membrane inserted ends of gp41, and the associated viral and cellular membranes, leading to merger and pore formation (see 20, 21, 22, 23, 24 in Bibliography 2).

How conformational signals are transmitted between receptor-bound gp120 and gp41 to trigger the refolding of gp41 into the fusion-active state is being elucidated. A gp120-gp41 association site formed by the terminal segments of C1 and C5 of gp120 and the central DSR of gp41 (see 25, 26, 27, 28 in Bibliography 2) may play an important role in this process as mutations in the DSR can inhibit CD4-triggered gp41 prehairpin formation and the initial hemifusion event (see 29 in Bibliography 2). Furthermore, the introduction of Cys residues to C5 and to the DSR generates an inactive disulfide-linked gp120-gp41 complex that is converted to a fusion-competent form by reduction (see 30, 31 in Bibliography 2). These findings implicate the C1-C5-DSR synapse in maintaining gp120-gp41 in the prefusion state and in subsequent transmission of fusion activation signals emanating from receptor-bound gp120. The terminal C1 and C5 gp41-contact regions project ˜35-Å from a 7-stranded β-sandwich at the base of the gp120 inner domain (8 in Bibliography 2). This β-sandwich appears to also play an important role in conformational signalling between gp120 and gp41 by linking CD4-induced structural changes in three structural layers of gp120 that emanate from the β-sandwich to gp41 activation (32 in Bibliography 2).

Understanding how conserved functional determinants of the HIV-1 glycoproteins tolerate or adapt to the rapid evolution of other Env regions is important for their evaluation and exploitation as potential drug and/or vaccine targets. For example, mutations in the DSR confer resistance to a novel low molecular weight fusion inhibitor, PF-68742, implicating this gp41 region as an inhibitor target (33 in Bibliography 2). Neutralizing antibodies exert strong evolutionary pressures on Env that can result in an increase in the number and/or a change in the position of potential N-linked glycosylation sites (PNGSs) that modify NAb-Env interactions (34, 35 in Bibliography 2). V1V2 is a key regulator of neutralization resistance, which generally correlates with its elongation and acquisition of PNGSs (34, 36, 37, 38, 39, 40, 41, 42, 43, 44 in Bibliography 2). Previously, the C1-C5-DSR association site was identified as a conserved determinant that exhibits structural and functional plasticity. This idea is based on the finding that whereas the overall gp120-gp41 association function of the DSR is conserved, the contribution of individual DSR residues to gp120 anchoring and membrane fusion function varies among HIV-1 strains and is controlled by V1V2 and V3 (28 in Bibliography 2. It is proposed that this plasticity enables the maintenance of a functional glycoprotein complex in a setting of host selective pressures that drive the rapid coevolution of gp120 and gp41.

Reference herein “MPER” means the conserved 23-residue Trp-rich sequence that connects the helical region 2 (HR2) of the gp41 ectodomain to the transmembrance domain.

Env immunogens may be produced by recombinant means typically in eukaryotic cells using methods known in the art. Eukaryotic cells include mammalian, plant, yeast and insect cells as known in the art. Recombinant proteins are produced by culturing the host cells for a period of time sufficient to allow for expression of the protein in the host cells or, more preferably, secretion of the protein into the culture medium in which the host cells are grown.

“Recombinant host cells”, “host cells”, “cells”, “cell lines”, “cell cultures”, and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected.

Suitable mammalian cell lines include, but are not limited to, BHK, VERO, HT1080, 293, 293T, 293F, RD, COS-7, CHO, Jurkat, HUT, SUPT, C8166, MOLT4/clone8, MT-2, MT-4, H9, PM1, CEM, myeloma cells (e.g., SB20 cells) and CEMX174 are available, for example, from the ATCC. Other host cells include without limitation yeast, e.g. Pichia pastoris, or insect cells such as Sf9 cells.

Synthetic DNA may be recombinantly expressed by molecular cloning into an expression vector containing a suitable promoter and other appropriate transcription regulatory elements, and transferred into prokaryotic or eukaryotic host cells to produce recombinant protein. Techniques for such manipulations are described by Sambrook et al., Molecular Cloning: A Laboratory Manual (2nd ed.). Cold Spring Harbour Laboratory, Cold Spring Harbour, N Y, 1989; Ausubel et al., Current Protocols in Molecular Biology, Green Pub. Associates and Wiley-Interscience, New York, 1988.

For example, a construct for expression in yeast preferably contains a synthetic gene, with related transcriptional and translational control sequences operatively linked to it, such as a promoter (such as GAL10, GALT, ADH1, TDH3 or PGK), and termination sequences (such as the S. cerevisiae ADH1 terminator). The yeast may be selected from the group consisting of: Saccharomyces cerevisiae, Hansenula polymorpha, Pichia pastoris, Kluyveromyces Kluyveromyces lactis, and Schizosaccharomyces pombe. See also Yeast Genetics: Rose et al., A Laboratory Course Manual, Cold Spring Harbor Laboratory, Cold Spring Harbor, N. Y., 1990. Nucleic acid molecules can be codon optimized for expression in yeast as known in the art (see Sharp and Cowe, Yeast, 7: 657-678, 1991). Appropriate vectors and control elements for any given cell type can be selected by one having ordinary skill in the art in view of the teachings of the present specification and information known in the art about expression vectors.

Vectors available for cloning and expression in host cell lines are well known in the art, and include but are not limited to vectors for cloning and expression in mammalian cell lines or yeast (fungal) cells, vectors for cloning and expression in bacterial cell lines, vectors for cloning and expression in phage and vectors for cloning and expression in insect cell lines. The expressed proteins can be recovered using standard protein purification methods.

Translational control elements have been reviewed by M. Kozak (e.g., Kozak, Mamm Genome, 7(8): 563-74, 1996; Kozak, Biochimie., 76(9): 815-21, 1994; Kozak, J Cell Biol, 108(2): 229-241, 1989; Kozak and Shatkin, Methods Enzymol, 60: 360-375, 1979).

Recombinant glycoproteins can be conveniently prepared using standard protocols as described for example in Sambrook, et al., 1989 (supra), in particular Sections 13, 16 and 17; Ausubel et al., Current Protocols in Molecular Biology, John Wiley & Sons Inc, 1994, in particular Chapters 10 and 16; and Coligan et al., 1995-1997 (supra), in particular Chapters 1, 5 and 6. The polypeptides or polynucleotides may be synthesized by chemical synthesis, e.g., using solution synthesis or solid phase synthesis as described, for example, in Chapter 9 of Atherton and Shephard, Peptide Synthesis. In Nicholson ed., Synthetic Vaccines, published by Blackwell Scientific Publications, and in Roberge et al., Science, 269(5221): 202-204, 1995.

In some embodiments, the modified Env immunogen is provided in a lipid containing vehicle such as a virus-like particle (VLP) or other lipid containing vehicle.

In one embodiment the specification provides a modified HIV envelope glycoprotein (Env) antigen or a lipid containing vehicle comprising same wherein the Env antigen comprises one of: (i) a second site suppressor mutation in residue 674 of the membrane proximal ectodomain region (MPER) of HIV gp41; (ii) a second site suppressor mutation which ablates a glycosylation site in the variable region (V1) region of gp120; or (iii) a second site suppressor mutation ablating a glycosylation site in the V1 region of gp120 and a second site suppressor mutation in residue 674 of the MPER of HIV gp41.

In another embodiment, the present invention provides a modified HIV Env immunogen or a lipid containing vehicle comprising same wherein the Env immunogen comprises one of: (i) a gp120-gp41 association site mutation or a reversion or pseudoreversion mutation thereof and/or a second site suppressor mutation in MPER; (ii) a gp120-gp41 association site mutation or a reversion or pseudoreversion mutation thereof and/or a second site suppressor mutation in a glycosylation site in the V1 region; or (iii) a gp120-gp41 association site mutation or a reversion or pseudoreversion mutation thereof and/or a second site suppressor mutation in a glycosylation site in the V1 region and a second site suppressor mutation in MPER.

In some embodiments, the gp120-gp41 association site mutation in the modified Env antigen is a DSR mutation or a reversion thereof or a pseudoreversion. In some embodiments, the reversion or pseudoreversion mutation permits cell to cell transmission competence in an intact viral particle. In some embodiments, the DSR mutation, reversion or pseudoreversion is K601H/N/K, i.e. to lysine, histidine or asparagine. Lysine is the wild-type residue, and histidine or asparagine are pseudoreversions. In some embodiments the W596L mutation is retained and allows cell to cell transmission in the context of K601H and D674E or ΔN¹⁷⁹ INN.

In some embodiments, the MPER mutation is in D674E of HIV gp41.

In some embodiments, the glycosylation mutation in V1 of HIV is gp120 the Env antigen or lipid vehicle of any one of claims 1 to 4 wherein the glycosylation site mutation in V1 of HIV gp120 is loss of one or two potential N-linked glycosylation sites in asparagine(s) 141 and/or 142 of AD8 or a corresponding mutation in other HIV strains.

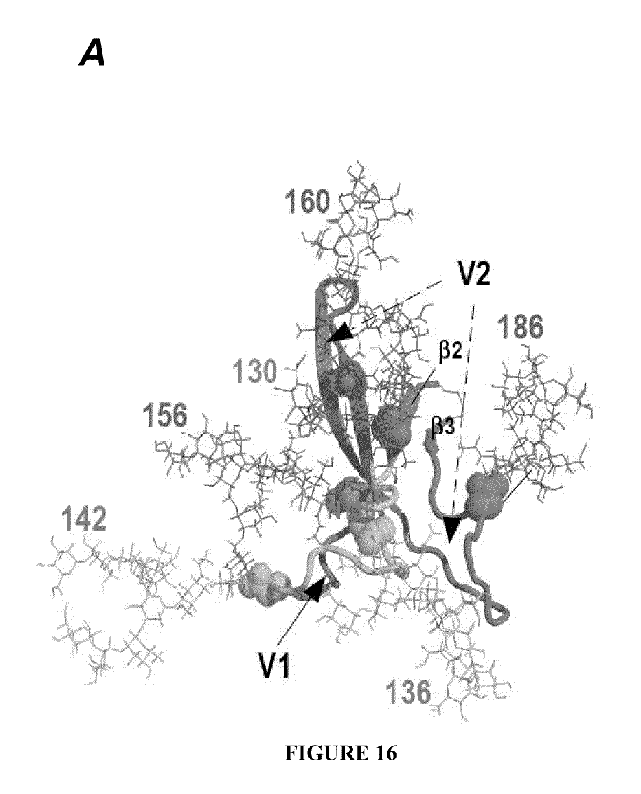

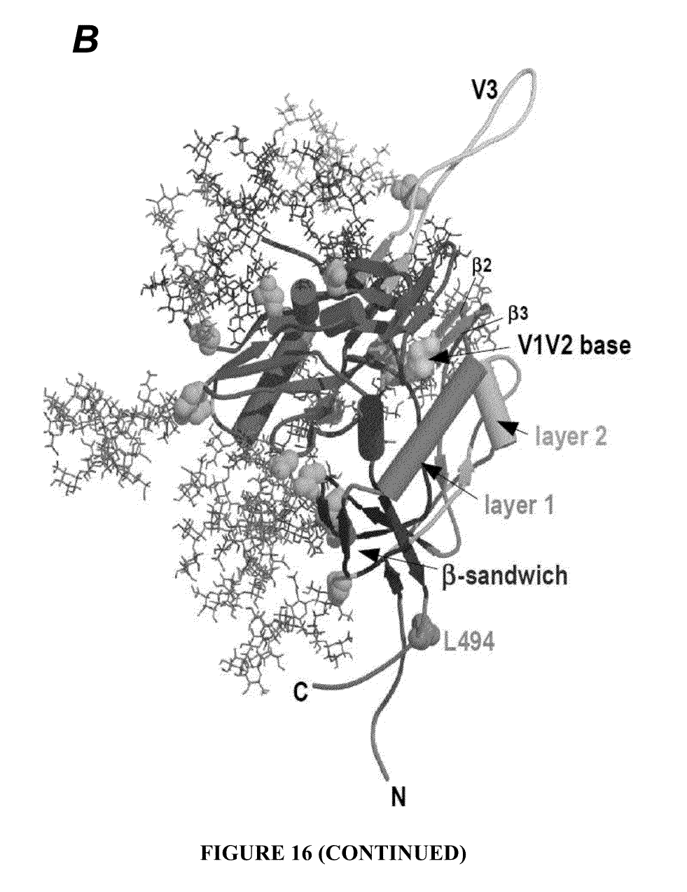

In some embodiments, the glycosylation mutation in V1 of HIV is the Env antigen or lipid vehicle of any one of claims 1 to 4 wherein the glycosylation site mutation in V1 of HIV gp120 is ΔN139INN or a corresponding deletion of conserved asparagine(s) in other HIV strains. See FIG. 16 for an alignment of V1 from different strains.

In some embodiments, mutation in V1 of HIV gp120 is ΔN¹³⁹INN.

In some embodiments the mutation in V1 is a glycosylation site mutation.

In some embodiments, the present invention provides a composition comprising a remodelled Env immunogen as described herein and a pharmaceutically or physiologically acceptable carrier or diluent.

Pharmaceutical compositions are conveniently prepared according to conventional pharmaceutical compounding techniques. See, for example, Remington's Pharmaceutical Sciences, 18th Ed., Mack Publishing, Company, Easton, Pa., U.S.A., 1990. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral or parenteral. In some embodiments, the composition comprises an adjuvant.

The response of a mammalian subject to immunogens can be enhanced if they are administered as a mixture with one or more adjuvants. Immune adjuvants typically function in one or more of the following ways: (1) immunomodulation (2) enhanced presentation (3) CTL production (4) targeting; and/or (5) depot generation. Illustrative adjuvants include: particulate or non-particulate adjuvants, complete Freund's adjuvant (CFA), aluminium salts, emulsions, ISCOMS, LPS derivatives such as MPL and derivatives thereof such as 3D, mycobacterial derived proteins such as muramyl di- or tri-peptides, particular saponins from Quillaja saponaria, such as QS21 and ISCOPREP 703, ISCOMATRIX™ adjuvant, and peptides, such as thymosin alpha 1. An extensive description of adjuvants can be found in Cox and Coulter, “Advances in Adjuvant Technology and Application”, in Animal Parasite Control Utilizing Biotechnology, Chapter 4, Ed. Young, W. K., CRC Press 1992, and in Cox and Coulter, Vaccine 15(3): 248-256, 1997.

In some embodiments, the adjuvant is ISCOMATRIX.

In some embodiments, the composition is for use in therapy, such as HIV prophylaxis or treatment in a mammalian subject.

A mammalian subject for the purpose of treating an HIV infection includes a mammal including humans, primates, laboratory animals, domestic and farm animals, zoo, sport and pet animals. Preferably, the mammal is a human subject.

The term treatment refers to therapeutic measures taken to prevent or slow, reduce HIV infection and its associated disorders or symptoms or the risk of developing advanced symptoms of HIV infection, or reliance on cART, or reducing the side effects of cART by reducing the frequency with which cART medication must be taken to maintain low viral loads. The term refers to any measurable or statistically significant amelioration in at least some subjects in one or more symptoms of HIV infection, or in the risk of developing same or transmitting infection.

The term prophylaxis or prevention and the like include administration of a composition as described herein to a subject not known to be infected with HIV for the purpose of preventing or attenuating an infection or reducing the risk of becoming infected or reducing the severity or onset of a condition or signs of a condition associated with HIV infection such as AIDS or an AIDs related condition or infection.

The administration of the vaccine composition is generally for prophylactic purposes. The prophylactic administration of the composition serves to prevent or attenuate any subsequent infection. A “pharmacologically acceptable” composition is one tolerated by a recipient patient. It is contemplated that an effective amount of the vaccine is administered. An “effective amount” is an amount sufficient to achieve a desired biological effect such as to induce enough humoral or cellular immunity. This may be dependent upon the type of vaccine, the age, sex, health, and weight of the recipient. Examples of desired biological effects include, but are not limited to, production of no symptoms, reduction in symptoms, reduction in virus titre, complete or partial protection against infection by HIV. The terms “effective amount” including “therapeutically effective amount” and “prophylactically effective amount” as used herein mean a sufficient amount of a composition of the present invention either in a single dose or as part of a series or slow release system which provides the desired therapeutic, preventative, or physiological effect in some subjects. Undesirable effects, e.g. side effects, may sometimes manifest along with the desired therapeutic effect; hence, a practitioner balances the potential benefits against the potential risks in determining an appropriate “effective amount”. The exact amount of composition required will vary from subject to subject, depending on the species, age and general condition of the subject, mode of administration and the like. Thus, it may not be possible to specify an exact ‘effective amount’. However, an appropriate ‘effective amount’ in any individual case may be determined by one of ordinary skill in the art using routine skills or experimentation. One of ordinary skill in the art would be able to determine the required amounts based on such factors as prior administration of the compositions or other agents, the subject's size, the severity of a subject's symptoms or the severity of symptoms in an infected population, viral load, and the particular composition or route of administration selected.

In some embodiments, a vaccine or composition of the present invention is physiologically significant if its presence results in a detectable change in the physiology of a recipient patient that enhances or indicates an enhancement in at least one primary or secondary humoral or cellular immune response against at least one strain of HIV. The vaccine composition is administered to protect against viral infection. The “protection” need not be absolute, i.e., the HIV infection need not be totally prevented or eradicated, if there is a statistically significant improvement compared with a control population or set of patients. Protection may be limited to reducing the severity or rapidity of onset of symptoms of the HIV infection.

In one embodiment, a vaccine composition of the present invention is provided to a subject either before the onset of infection (so as to prevent or attenuate an anticipated infection) or after the initiation of an infection, and thereby protects against viral infection. In some embodiments, a vaccine composition of the present invention is provided to a subject before or after onset of infection, to reduce viral transmission between subjects.

It will be further appreciated that compositions of the present invention can be administered as the sole active pharmaceutical agent, or used in combination with one or more agents to treat or prevent HIV or symptoms associated with HIV infection.

In another embodiments, the present invention provides the composition in, or in the manufacture of a medicament for, the treatment or prevention of HIV infection.

In another embodiment, the present invention provides for use of the composition in, or in the manufacture of a diagnostic agent for, the diagnosis or monitoring of HIV infection or for monitoring an anti HIV treatment protocol.

In some embodiments, the diagnostic agent is an antibody or comprises an antigen binding fragment thereof.

The present invention, an a related embodiment provides, a method of eliciting an immune response in a mammalian subject, the method comprising administering an effective amount of the composition as described herein for a time and under conditions sufficient to elicit an immune response.

Administration of the herein described HIV Env composition or a antibody determined thereby is generally for a time and under conditions sufficient to elicit an immune response comprising the generation of neutralizing antibodies. The immunogenic compositions may be administered in a convenient manner such as by the pulmonary, oral, intravenous (where water soluble), intraperitoneal, intramuscular, subcutaneous, intradermal, intrathecal or suppository routes or implanting (e.g. using slow release formulations). Administration may be systemic or local, although systemic is more convenient. Other contemplated routes of administration are by patch, cellular transfer, implant, sublingually, intraocularly, topically, orally, rectally, vaginally, nasally or transdermally.

As used herein, an “immune response” refers to the reaction of the body as a whole to the presence of a composition of the present invention which includes making antibodies and developing immunity to the composition. Therefore, an immune response to an immunogen also includes the development in a subject of a humoral and/or cellular immune response to the immunogen of interest. A “humoral immune response” is mediated by antibodies produced by plasma cells. A “cellular immune response” is one mediated by T lymphocytes and/or other white blood cells. As used herein, “antibody titres” can be defined as the highest dilution in post-immune sera that resulted in a value greater than that of pre-immune samples for each subject.

The assays for assessing immune responses may comprise in vivo assays, such as assays to measure antibody responses, neutralisation assays and delayed type hypersensitivity responses. In an embodiment, the assay to measure antibody responses primarily may measure B-cell function as well as B-cell/T-cell interactions. For the antibody response assay, antibody titres in the blood may be compared following an antigenic challenge. These levels can be quantitated according to the type of antibody, as for example, IgG, IgG1, IgG2, IgG3, IgG4, IgM, IgA or IgD. Also, the development of immune systems may be assessed by determining levels of antibodies and lymphocytes in the blood without antigenic stimulation. The assays may also comprise in vitro assays. The in vitro assays may comprise determining the ability of cells to divide, or to provide help for other cells to divide, or to release lymophokines and other factors, express markers of activation, and lyse target cells. Lymphocytes in mice and man can be compared in in vitro assays. In an embodiment, the lymphocytes from similar sources such as peripheral blood cells, splenocytes, or lymph node cells, are compared. It is possible, however, to compare lymphocytes from different sources as in the non-limiting example of peripheral blood cells in humans and splenocytes in mice. For the in vitro assay, cells may be purified (e.g., B-cells, T-cells, and macrophages) or left in their natural state (e.g., splenocytes or lymph node cells). Purification may be by any method that gives the desired results. The cells can be tested in vitro for their ability to proliferate using mitogens or specific antigens. The ability of cells to divide in the presence of specific antigens can be determined using a mixed lymphocyte reaction (MLR) assay. Supernatant from the cultured cells can be tested to quantitate the ability of the cells to secrete specific lymphokines. The cells can be removed from culture and tested for their ability to express activation antigens. This can be done by any method that is suitable as in the non-limiting example of using antibodies or ligands which bind to the activation antigen as well as probes that bind the RNA coding for the activation antigen. Also, in an embodiment, phenotypic cell assays can be performed to determine the frequency of certain cell types. Peripheral blood cell counts may be performed to determine the number of lymphocytes or macrophages in the blood. Antibodies can be used to screen peripheral blood lymphocytes to determine the percent of cells expressing a certain antigen as in the non-limiting example of determining CD4 cell counts and CD4/CD8 ratios.

In accordance with these embodiments, the composition is preferably administered for a time and under conditions sufficient to elicit an immune response comprising the generation of Env-specific neutralizing antibodies. The compositions of the present invention may be administered as a single dose or application. Alternatively, the compositions may involve repeat doses or applications, for example the compositions may be administered 2, 3, 4, 5, 6, 7, 8, 9, 10 or more times over relatively long periods in order to enable B-cell maturation and somatic mutation of antibody genes and engender neutralizing antibodies. In some embodiments, the immune response comprises the production of neutralizing antibodies.

In other embodiments, the present invention provides a method of immunising a subject against an HIV infection comprising administering the composition as described herein to the mammalian subject.

In some embodiments, a method of treating or preventing an HIV infection in a mammalian subject is provided, said method comprising administering the composition comprising a remodelled Env immunogen as described herein to the subject for a time and under conditions sufficient to treat an HIV infection in the subject.

Also contemplated are a kit or solid substrate comprising a remodelled Env immunogen or lipid containing particle comprising same as described herein.

The invention provides an Env antigen or a nucleic acid molecule encoding same identified by the process described herein for identifying, producing or selecting modified Env immunogens.

In a further aspect, the present invention provides a process for producing a neutralizing antibody comprising injecting into a subject an immunologically effective amount of the modified Env composition of the present invention, and isolating and purifying the antibody produced. In another embodiment, the present invention provides purified antibodies raised against one or more of the subject HIV Env-based compositions described herein. Preferably, neutralizing antibodies are broadly neutralizing antibodies which neutralize more than one HIV virus from different clades.

Antibodies may be polyclonal or monoclonal. Further, antibodies may be selected for diagnostic, prognostic, therapeutic, prophylactic, and screening purposes typically using criteria known to those of skill in the relevant art. In some embodiments, neutralization potency and cross-clade and cross-Env specificity neutralization ability is tested relative to suitable controls to identify antibodies with superior ability. In other embodiments, cell binding analysis may be performed. Antibodies may be tested on multi-clade pseudovirus panels of hundreds of HIV viruses to assess neutralization breadth and potency. Neutralization breadth may be determined as a percent neutralization with an IC50 or IC90 of less than about 2 ug/ml to less than about 0.1 ug/ml to less than 0.01 ug/ml. Recombinant rescue of monoclonal antibodies may involve the use of B-cell culture systems as described previously. Antibodies may be tested before and after deglycosylation of gp120.

The terms “antibody” and “antibodies” include polyclonal and monoclonal antibodies and all the various forms derived from monoclonal antibodies, including but not limited to full-length antibodies (e.g. having an intact Fc region), antigen-binding fragments, including for example, Fv, Fab, Fab′ and F(ab′)₂ fragments; and antibody-derived polypeptides produced using recombinant methods such as single chain antibodies. The terms “antibody” and “antibodies” as used herein also refer to human antibodies produced for example in transgenic animals or through phage display, as well as antibodies, human or humanized antibodies, primatized antibodies or deimmunized antibodies. It also includes other forms of antibodies that may be therapeutically acceptable and antigen-binding fragments thereof, for example single domain antibodies derived from cartilagenous marine animals or Camelidae, or from libraries based on such antibodies. The selection of fragmented or modified forms of the antibodies may also involve consideration of any affect the fragments or modified forms have on the half-lives of the antibody or fragment.

In some embodiments, the antibody is provided with a pharmaceutically or pharmacologically acceptable carrier, diluent or excipient.

In other embodiments, the antibody is selected for diagnosis or prognosis. In some embodiments, kits comprising antibodies determined by the modified Env glycoproteins of the present invention are contemplated.

A “pharmaceutically acceptable carrier and/or a diluent” is a pharmaceutical vehicle comprised of a material that is not otherwise undesirable i.e., it is unlikely to cause a substantial adverse reaction by itself or with the active composition. Carriers may include all solvents, dispersion media, coatings, antibacterial and antifungal agents, agents for adjusting tonicity, increasing or decreasing absorption or clearance rates, buffers for maintaining pH, chelating agents, membrane or barrier crossing agents. A pharmaceutically acceptable salt is a salt that is not otherwise undesirable. The agent or composition comprising the agent may be administered in the form of pharmaceutically acceptable non-toxic salts, such as acid addition salts or metal complexes.

For oral administration, the compositions can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. Tablets may contain a binder such as tragacanth, corn starch or gelatin; a disintegrating agent, such as alginic acid; and a lubricant, such as magnesium stearate. If desired, tablets may be sugar-coated or enteric-coated by standard techniques. The active composition can be encapsulated to make it stable to passage through the gastrointestinal tract. See for example, International Patent Publication No. WO 96/11698.

For parenteral administration, the composition may be dissolved in a carrier and administered as a solution or a suspension. For transmucosal or transdermal (including patch) delivery, appropriate penetrants known in the art are used for delivering the composition. For inhalation, delivery uses any convenient system such as dry powder aerosol, liquid delivery systems, air jet nebulizers, propellant systems. For example, the formulation can be administered in the form of an aerosol or mist. The compositions may also be delivered in a sustained delivery or sustained release format. For example, biodegradable microspheres or capsules or other polymer configurations capable of sustained delivery can be included in the formulation. Formulations can be modified to alter pharmacokinetics and biodistribution. For a general discussion of pharmacokinetics, see, e.g., Remington's Pharmaceutical Sciences, 1990 (supra). In some embodiments the formulations may be incorporated in lipid monolayers or bilayers such as liposomes or micelles. Targeting therapies known in the art may be used to deliver the agents more specifically to certain types of cells or tissues.

The actual amount of active agent administered and the rate and time-course of administration will depend on the nature and severity of the disease. Prescription of treatment, e.g. decisions on dosage, timing, etc. is within the responsibility of general practitioners or specialists and typically takes into account the condition of the individual patient, the site of delivery, the method of administration and other factors known to practitioners. Examples of techniques and protocols can be found in Remington's Pharmaceutical Sciences, 1990 (supra).

Sustained-release preparations that may be prepared are particularly convenient for inducing immune responses. Examples of sustained-release preparations include semipermeable matrices of solid hydrophobic polymers containing the polypeptide, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers, and poly-D-(−)-3-hydroxybutyric acid. While polymers such as ethylene-vinyl acetate and lactic acid-glycolic acid enable release of molecules for over 100 days, certain hydrogels release proteins for shorter time periods. Liposomes may be used which are of the small (about 200-800 Angstroms) unilamellar type in which the lipid content is greater than about 30% cholesterol, the selected proportion being adjusted for the optimal therapy.

Stabilization of proteins may be achieved by modifying sulfhydryl residues, lyophilizing from acidic solutions, controlling moisture content, using appropriate additives, and developing specific polymer matrix compositions. The in vivo half life of proteins may be extended using techniques known in the art, including, for example, by the attachment of other elements such as polyethyleneglycol (PEG) groups.

Prime-boost immunization strategies as disclosed in the art are contemplated. See for example International Publication No. WO/2003/047617. Thus, compositions may be in the form of a vaccine, vector, DNH priming or boosting agent.

In some embodiments, kits comprising the herein described modified Enc glycoprotein are conveniently used for (or are for use in) diagnosis or prognosis of viral infection, or pathogen monitoring or serosurveillance kits, and optionally include packaging, instructions and various other components such as buffers, substrates, antibodies or ligands, control antibodies or ligands, and detection reagents.

The term “isolated” and “purified” means material that is substantially or essentially free from components that normally accompany it in its native state. For example, an “isolated nucleic acid molecule” refers to a nucleic acid or polynucleotide, isolated from the sequences which flank it in a naturally-occurring state, e.g., a DNA fragment which has been removed from the sequences that are normally adjacent to the fragment. In particular, an isolated Env includes in vitro isolation and/or purification of a protein from its natural cellular environment, or from association with other components of a cell. Without limitation, an isolated nucleic acid, polynucleotide, peptide, or polypeptide can refer to a native sequence that is isolated by purification or to a sequence that is produced by recombinant or synthetic means.

Reference to variants includes mutations, parts, derivatives and functional analogs. While the mutations described in the Examples were selected for by forced evolution, further mutants may be included using an iterative approach. In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site. Amino acid sequence deletions generally range from about 1 to 15 residues, more preferably about 1 to 10 residues and typically about 1 to 4, 1 to 5 or 1 to 10, or 1 to 15 or 1 to 20 contiguous amino acid residues. Substitution mutants have at least one amino acid residue in the polypeptide molecule removed and a different residue inserted in its place. The sites of greatest interest for substitutional mutagenesis have been identified herein as the DSR, MPER and the V1 regions. Glycosylation variants can be assessed by known methods such as by mass spectrometry.

The present invention is further described by the following non-limiting Examples.

### Example 1

### Methods

### Env Expression Vectors and Proviral Clones

The CMV promoter-driven HIV-1 _{AD8} Env expression vector, pCDNA3.1-AD8env, is described elsewhere [33]. pΔKAD8env was derived by religation of the end-filled HindIII and EcoRI sites of pCDNA3.1-AD8env. Mutants of the pAD8 infectious clone (obtained from K. Peden [70]) were prepared by transferring the EcoRI-BspMI env-containing fragment from pCDNA3.1-AD8env vectors into pAD8. In vitro mutagenesis of the gp41 region was carried out using the Quikchange protocol (Stratagene).

### Infection of U87.CD4.CCR5 Cells

Virus stocks were prepared by transfecting 293T cell monolayers with pAD8 infectious clones using Fugene 6 or Fugene HD (Roche). Virus-containing transfection supernatants were normalized according to reverse transcriptase (RT) activity, and then used to infect U87.CD4.CCR5 astroglioma cells (from H. Deng and D. Littman [71], NIH AIDS Research and Reference Reagent Program) in 25 cm² culture flasks. The supernatants were assayed for RT activity at various time points. To assess the transmission of cell-associated viruses, HIV-1 particles were pseudotyped with VSV G by cotransfection of 293T cells with pAD8 and pHEF-VSV G (from Dr. L.-J. Chang [72] NIH AIDS Research and Reference Reagent Program). U87.CD4.CCR5 cells in 25 cm² culture flasks were inoculated with the HIV-VSV G pseudotypes, and then, at 24-h postinfection, trypsinized to remove surface-adsorbed virions. The cells were replated and then cultured for 10 days. The culture supernatants were assayed for RT activity at days 3, 7 and 10. For long-term cultures of viral mutants, the day-10 cell-free culture supernatants were filtered (0.45 μm pore size) and normalized according to RT activity prior to the next passage (5 passages in total). Genomic DNA was extracted from infected cells using Qiagen DNeasy Blood and Tissue kit. The viral DNA fragment encompassed by nucleotides 5954-9096 (HIV-1_{HXB2R} numbering convention) was PCR-amplified using Expand HiFi (Roche) and the primers, 5′-GGCTTAGGCATCTCCTATGGCAGGAAGAA, SEQ ID NO:1, (Env1A) and 5′-TAGCCCTTCCAGTCCCCCCTTTTCTTTTA, SEQ ID NO:2, (Env1M) [73]. The amplified sequences were ligated into pΔKAD8env (KpnI-XbaI) and the entire env open reading frame sequenced using ABI BigDye terminator v3.1.

### Single Cycle Infectivity Assays

Single cycle infectivity assays were conducted as described [24]. Env-pseudotyped luciferase reporter viruses were produced by cotransfecting 293T cells with pΔKAD8env plus the luciferase reporter virus vector, pNL4.3.Luc.R⁻E⁻ (NIH AIDS Research and Reference Reagent Program, from N. Landau [74]), using Fugene HD. The infectivity of pseudotyped viruses was determined in U87.CD4.CCR5 cells using the Promega luciferase assay system at 48 h postinfection.

### Western Blotting

Twenty four h after transfection with pΔKAD8env vectors, 293T cells were lysed for 10 min on ice in PBS containing 1% Triton X-100, 0.02% sodium azide, 1 mM EDTA. The lysates were clarified by centrifugation for 10 min at 10,000×g at 4° C. prior to SDS-PAGE under reducing conditions. The proteins were transferred to nitrocellulose and blotted with antibodies C8 to gp41 [75] and DV-012 to gp120 [76] (from G. Lewis and M. Phelan, respectively, NIH AIDS Research and Reference Reagent Program). The immunoblots were developed with Alexa Fluor 680-conjugated goat anti-mouse or donkey anti-sheep immunoglobulin (Invitrogen) and scanned in a LI-COR Odyssey infrared imager. For virion analysis, supernatants from pAD8-transfected 293T cells were centrifuged over 1.5 ml 25% w/v sucrose/PBS cushions (Beckman SW41 Ti rotor, 25,000 rpm, 2.5 h, 4° C.) prior to reducing SDS-PAGE and western blotting with DV-012 to detect gp120 and pooled IgG from HIV-1-infected individuals to detect Gag proteins.

### Biosynthetic Labelling and Immunoprecipitation

293T cells were transfected with pΔKAD8env vectors. At 24-h post transfection, the cells were incubated for 30 min in cysteine and methionine-deficient medium (MP Biomedicals), and then labelled for 45 min with 150 μCi Tran-³⁵S-label (MP Biomedicals). The cells were washed and then chased in complete medium for 5 h prior to lysis. Cell lysates and clarified culture supernatants were immunoprecipitated with pooled IgG from HIV-1-infected persons and protein G Sepharose and subjected to SDS-PAGE in the presence of β-mercaptoethanol. The labelled proteins were visualized by scanning in a Fuji phosphorimager.

### Luciferase Reporter Assay of Cell-Cell Fusion

Cell-cell fusion assays were conducted as previously described [24]. Briefly, 293T cells were cotransfected with pΔKAD8env and the bacteriophage T7 RNA polymerase expression vector, pCAG-T7 [77]. BHK21 target cells were cotransfected with pc.CCR5 (AIDS Research and Reference Reagent Program from N. Landau [78]) and pT4luc, a bicistronic vector that expresses human CD4 from a CMV promoter and firefly luciferase from a T7 promoter [13]. At 24 h posttransfection, targets and effectors were cocultured in triplicate in a 96-well plate (18 h, 37° C.) and then assayed for luciferase activity (SteadyGlo, Promega).

### Neutralization Assay

Purified IgG of brNAbs 2F5 [63], 4E10 [64] and IgGb12 [79, 80] were obtained from Polymun Scientific, while the HR2 peptide analogue, C34 [40], was purchased from Genscript. Neutralization assays were conducted using TZM-bl cells (obtained from J. C. Kappes, X. Wu and Tranzyme Inc., NIH AIDS Research and Reference Reagent Program [81-83]), a HeLa cell line expressing CD4 and CCR5 and harbouring integrated copies of the luciferase and β-galactosidase genes under control of the HIV-1 promoter. Virus stocks produced by pAD8-transfected 293T cells and determined to give ˜1.5×10⁶ relative light units (RLU) following infection of TZM-bl cells, were mixed with an equal volume of serially diluted IgG or C34 peptide and incubated for 1H at 37° C. One hundred μl of the virus-IgG mixture was then added to TZM-bl cells (10⁴ cells in 100 μl per well of a 96-well tissue culture plate) and incubated for 2 days prior to lysis and assay for luciferase activity (Promega, Madison, Wis.). For experiments with the CCR5 antagonist maraviroc (NIH AIDS Research and Reference Reagent Program [84]), the cells were preincubated for 1 h at 37° C. with the drug prior to incubation with virus for 48 h. Neutralizing activities were measured in triplicate and reported as the average percent luciferase activity.

### Phenotype of WL/KD

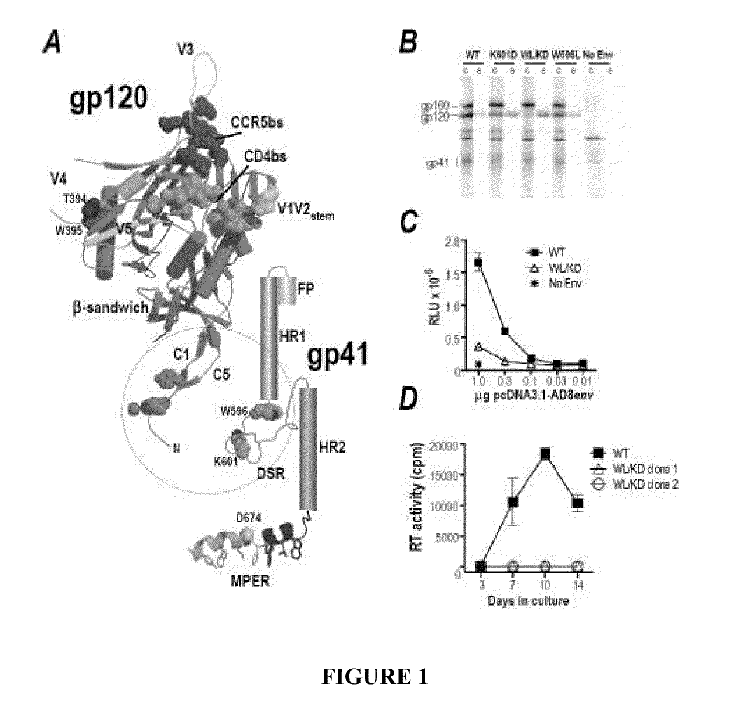

The gp120-gp41 association phenotype of the WL/KD mutant was investigated by immunoprecipitation of biosynthetically labelled Env glycoproteins expressed in 293T cells [33]. The WL/KD mutation led to >95% of total gp120 being sloughed into the culture supernatant (FIG. 1B) indicating a shedding phenotype that was more severe than those of the component single K601D and W596L mutants. The loss of gp120-gp41 association for WL/KD corresponded with the inhibition of cell-cell fusion function in a luciferase reporter assay employing Env-293T effector cells and CD4 and CCR5-expressing BHK21 targets (FIG. 1C). Consistent with these gp120-shedding and fusion defects, WL/KD blocked HIV-1_{AD8} viral replication in U87.CD4.CCR5 cells (FIG. 1D).

Long-Term Culture of HIV-1_{AD8}-WL/KD.

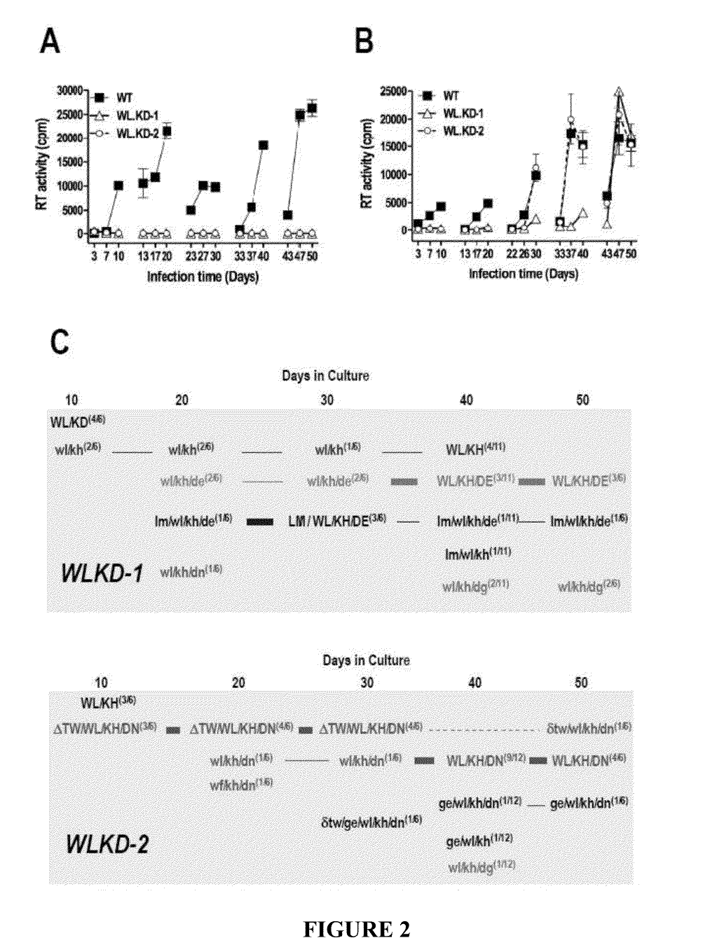

Viruses derived from 2 independent HIV-1_{AD8} proviral clones carrying WL/KD were subjected to long-term culture in U87.CD4.CCR5 cells with serial passaging of cell-free virus onto fresh cells every 10 days. Evidence of replication was not observed for either clone, even after 50 days of culture (FIG. 2A). The markedly diminished gp120-anchoring ability of WL/KD gp41 was assumed to have blocked viral entry and therefore reverse transcription, which is required for the generation of suppressor mutations. Mutant WL/KD HIV-1 particles were therefore pseudotyped with vesicular stomatitis virus glycoprotein G (VSV G) in trans in order to initiate HIV-1 envelope glycoprotein (Env)-independent infection via the endosomal pathway. Twenty-four h after infection, the U87.CD4.CCR5 cells were extensively washed and trypsinized to remove residual adsorbed virus prior to further culture for 10 days. The sequential passaging of the resultant cell-free virus in U87.CD4.CCR5 cells led to restored infectivity after 47 and 30 days in WL/KD cultures 1 and 2 (WLKD-1 and WLKD-2), respectively (FIG. 2B).

The env region was PCR-amplified from genomic DNA isolated at days 10, 20, 30, 40 and 50, the PCR products were cloned into pΔKAD8env, and the entire env region was sequenced. WLKD-1. A D601H pseudoreversion emerged at day 10 (2/6 clones, WL/KH) prior to the appearance of D674E in the MPER at day 20, which persisted throughout the culture period. The genotypes observed over the 50-day culture period included WL/KH (9/35 clones), W596L/K601H/D674E (WL/KH/DE [10/35 clones]), L85M/W596L/K601H/D674E (LM/WL/KH/DE [6/35 clones]), W596L/K601H/D674G (WL/KH/DG [4/35 clones]), L85M/W596L/K601H (LM/WL/KH [1/35 clones]), and W596L/K601H/D674N (WL/KH/DN [1/35 clones]) (FIG. 2C). WLKD-2. At day 10, 3/6 clones contained WL/KH, while 3 others contained the Thr-394-Trp-395 deletion in V4 (ATW), together with W596L and D601H in the DSR, and D674N in the MPER (ATW/WL/KH/DN). The ATW/WL/KH/DN genotype persisted to day 30 but at days 40 and 50, the dominant genotype was WL/KH/DN (13/18 clones) (FIG. 2C). The W596L mutation was retained in 70/71 env clones obtained from the WLKD-1 and WLKD-2 cultures indicating a strong selection pressure to maintain Leu at 596. The K601H and D674E mutations were not observed in env clones obtained following passaging of the WT virus, while D674N was observed in 1 clone (data not shown).

### Infectivity of WL/KD Revertants.

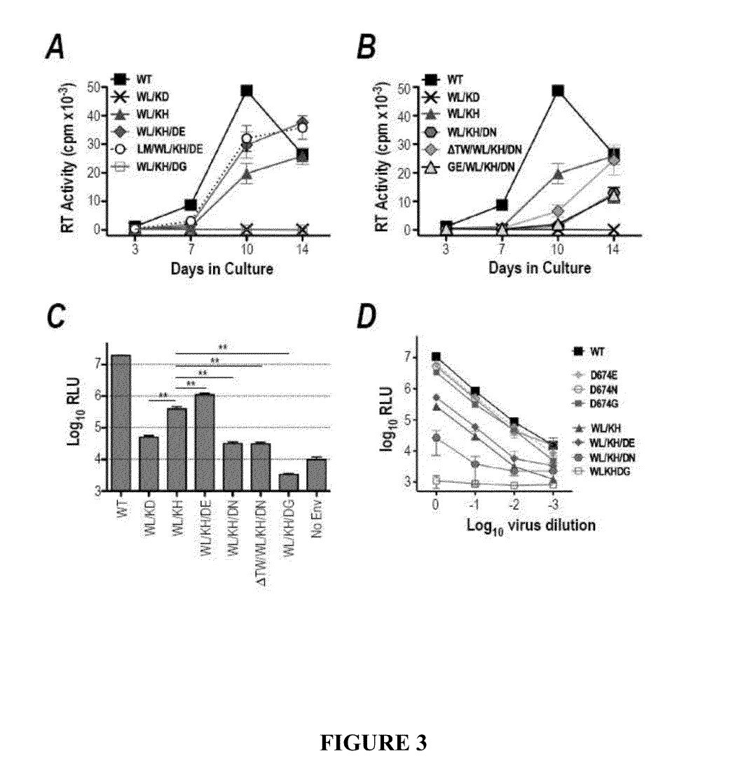

The dominant genotypes were reconstructed in the context of the pAD8 proviral clone. In the case of WLKD-1, cell-free virus-initiated replication in U87.CD4.CCR5 cells was partially restored by D601H in the DSR (WL/KH) and was optimised further by D674E in the MPER (WL/KH/DE) (FIG. 3A). The addition of L85M to WL/KH/DE did not improve replication any further. Interestingly, the WL/KH/DG combination was replication-incompetent. Gly-674 can arise via an A-to-G mutation in the 2^{nd} position of the Asp and Glu codons but in a WL/KH context appears to be an evolutionary dead-end. These data suggest that D601H and D674E can act synergistically to suppress the original replication defect. For WLKD-2, step-wise improvements in replication competence were observed with WL/KH/DN, ATW/WL/KH/DN and WL/KH, respectively (FIG. 3B). Thus D674N is inhibitory to cell-free virus initiated replication in the context of WL/KH with ATW partially relieving this inhibition. The G145E V1 mutation observed at days 30-40 did not confer a replication advantage to WL/KH/DN. Interestingly, the replication competence of WLKD-2 genotypes were inferior to those derived from the WLKD-1 culture even though revertant virus emerged in the WLKD-2 culture first, suggesting that additional mechanisms of reversion were operating in this culture.

The infectivity associated with revertant genotypes was further examined in a single cycle infectivity assay employing Env-pseudotyped luciferase reporter viruses. The infectivity of WL/KD for U87.CD4.CCR5 cellular targets was reduced by ˜2.5 log₁₀ with respect to WT (FIG. 3C). The D601H pseudoreversion in WL/KH increased this infectivity by ˜10-fold while the addition of D674E led to a further 2-fold improvement, but the entry competence of WL/KH/DE remained 20-fold lower than WT. The addition of D674G to WL/KH (WL/KH/DG) markedly suppressed viral entry consistent with the observed lack of replication. The alternate MPER mutation, D674N, was inhibitory on the WL/KH background, consistent with their relative replicative capacity, while the addition of ATW to WL/KH/DN did not improve single-cycle entry competence any further.

It was investigated whether the modulation of infectivity by D674E, D674N and D674G occurred via a functional link to Leu-596 and His-601 or whether it could be explained by a generalized enhancement or inhibition in Env function. FIG. 3D indicates that the D674 mutations did not alter the infectivity of Env-pseudotyped luciferase reporter virus when introduced to the WT background, indicating a specific functional interaction between Leu-596, His-601 in the DSR and position 674 in the MPER.

### Cell-Cell Spread of Revertant Viruses.

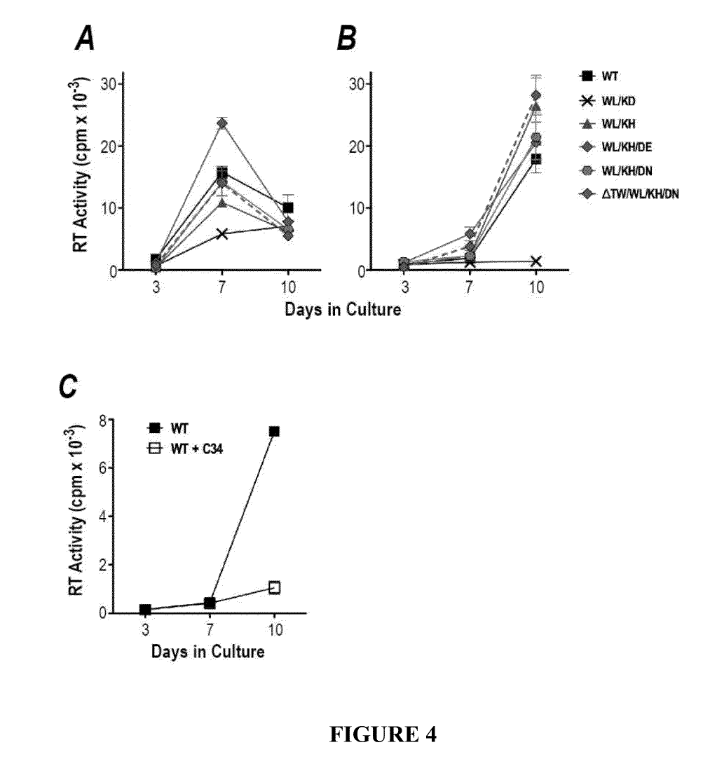

The functional advantages conferred by D601H and D674E were less obvious in the single cycle infectivity assay when compared to 14-day replication experiments (compare FIGS. 3A, B and C). This apparent discrepancy may be explained by the fact that only a single cycle of infection mediated by cell-free virus occurs in the reporter assay, whereas multiple rounds of infection mediated by both cell-free and cell-associated virus occur in the replication assay [34, 35]. U87.CD4.CCR5 cells were therefore inoculated with HIV-1-VSV G pseudotyped particles, reasoning that the highly fusogenic nature of VSV G will normalize the cellular entry of cell-free WT and revertant viruses in the first 24 hours of infection, thereby enabling an assessment of virus production following multiple rounds of cell-cell and cell-free viral transmission. At 24-h postinfection, the cells were trypsinized to remove residual surface-adsorbed virus, replated and then cultured for a further 10 days. Virus production was elevated for WL/KH/DE with respect to WT at day 7 and approached WT levels at day 10, whereas WL/KH, WL/KH/DN and ΔTW/WL/KH/DN replication was almost identical to WT over the 10-day culture (FIG. 4A). A low level of RT activity was observed for WL/KD, which is likely due to a combination of virus production by cells infected by VSV G-pseudotypes in the initial 24 h plus low-level cell-cell spread. The results were confirmed in an experiment employing smaller inocula (20,000 cpm RT activity-equivalents of VSV G-HIV-1 pseudotypes) but virus production was delayed to day 10 in this case (FIG. 4B). Notably, the presence of D674N was not inhibitory to replication when combined with WL/KH in this infection system. We found that viral spread in VSV G-HIV-1 pseudotype initiated cultures was blocked by the C34 fusion inhibitor peptide, consistent with viral spread being HIV-1 Env dependent (FIG. 4C). These data suggest that the D601H pseudoreversion and 2^{nd} (and 3^{rd}) site mutations optimise viral spread mediated by cell-associated virus.

### Glycoprotein Expression and Subunit Association.

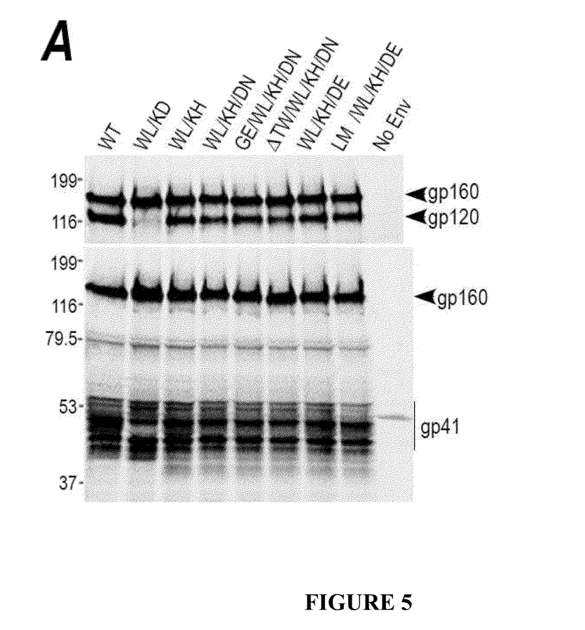

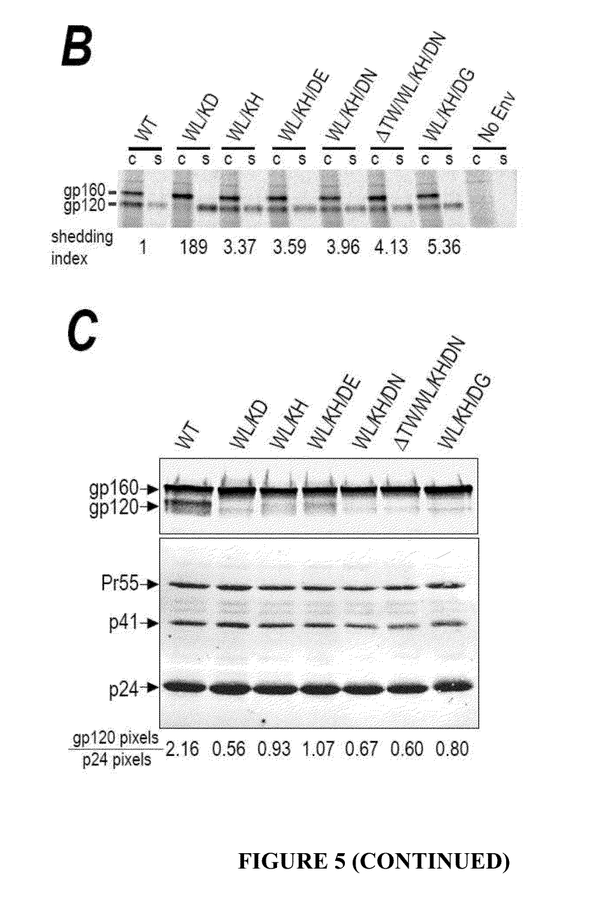

The synthesis and processing of the cloned Env glycoproteins were examined by western blot. The gp120-specific polyclonal antibody, DV012, (FIG. 5A, upper) revealed that similar levels of gp160 were expressed for all clones and that cell-associated gp120 was present for WT and His-601-containing clones. Consistent with the shedding defect seen previously (FIG. 1B), gp120 was largely absent for WL/KD. The gp41-directed monoclonal antibody (mAb) C8, revealed similar levels of gp160 and gp41 expression for the Env constructs examined (FIG. 5A, lower). Interestingly, the presence of the WL/KD mutation in gp41 resulted in a distinct glycosylation pattern relative to the other clones, suggesting a subtly different structure. The gp120 anchoring ability of the revertant Env proteins was confirmed by immunoprecipitation of pulse-chase biosynthetically labelled Env transfected 293T cells. FIG. 5B again confirms the gp120 shedding defect of WL/KD and indicates that the subsequent D601H mutation, present in the clone WL/KH was sufficient to partially restore gp120 association levels with no further improvements to association following the addition of 2^{nd} and 3^{rd} site mutations. Western blot analysis of viruses derived from pAD8 proviral clones revealed gp120 shedding phenotypes for the revertants (FIG. 5C).

### Examination of the 601-674 Functional Linkage in Cell-Cell Fusion.

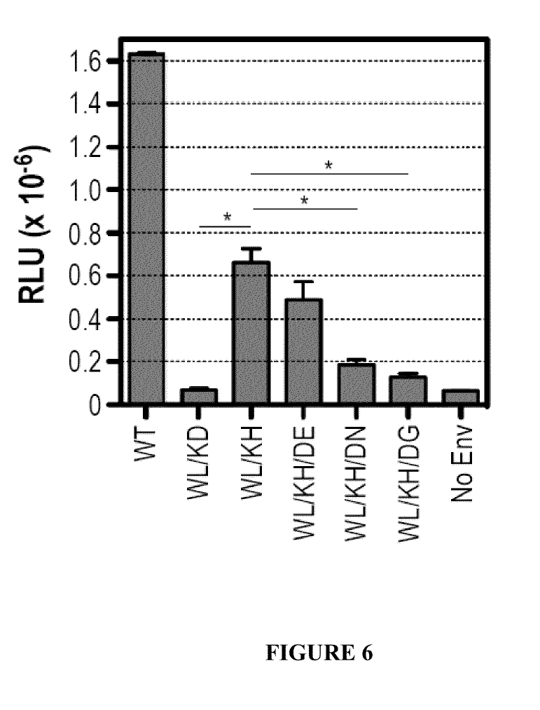

The membrane fusion activities of selected revertant Env sequences were examined in a cell-to-cell fusion assay. In this context, Env is expressed in the absence of other viral proteins and is therefore not subjected to the conformational constraints that may be imposed by matrix-gp41 cytoplasmic tail interactions present in virus [36-39]. The assay was conducted at limiting Env concentrations (0.25 μg pΔKADenv) to enable detection of subtle changes in fusion function. Consistent with the cell-free virus infectivity data, WL/KD blocked cell-cell fusion, WL/KH exhibited partially restored fusion function and D674N and D674G mutations were inhibitory in a WL/KH context (FIG. 6). However, in contrast to the infectivity data, D674E did not enhance fusogenicity when added to WL/KH. These data suggest that the functional interaction between Leu-596, His-601 and Glu-674 largely operates in the context of assembled virions transmitted via the cell-cell route and the conformational constraints imposed by Gag-gp41 cytoplasmic tail interactions [36-39].

### Neutralization Sensitivity of WL/KH and WL/KH/DE Mutants.

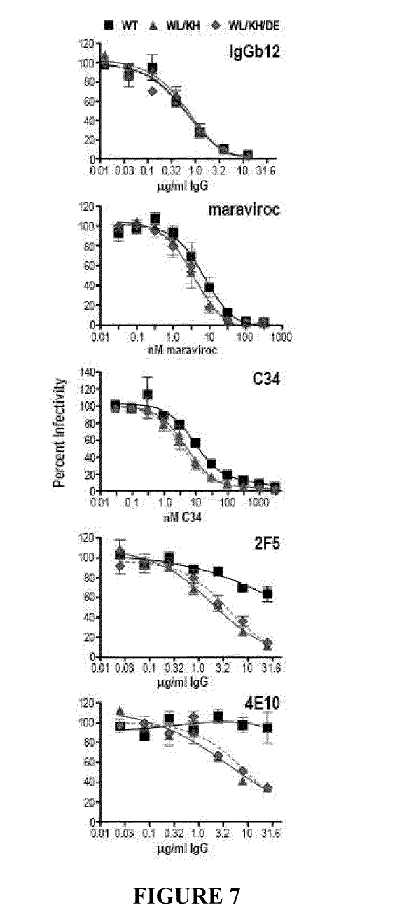

To determine whether WL/KH and WL/KH/DE are associated with structural changes in gp41, neutralizing agents were used to probe functional virion-associated gp120-gp41 complexes. Virus stocks, produced by transfecting 293T cells with pAD8 infectious clones, were adjusted to produce ˜1.5×10⁶ RLU following 48 h of infection of TZM-bl cells. The viruses were then incubated with the neutralizing agents for 1 h prior to infection of naïve TZM-b1 cells. In the case of maraviroc, target cells were pretreated with the CCR5 antagonist for 1 h prior to infection. The CD4 binding site brNAb, IgGb12, and the CCR5 antagonist, maraviroc, neutralized WT, WL/KH and WL/KH/DE to similar extents (the maraviroc IC₅₀ and IC₉₀ values for WT were not significantly different to those obtained with the mutants) indicating that gp120-CD4-CCR5 interactions had not been affected by the mutations in gp41 (FIG. 7). Small, but significant ˜0.5 log₁₀-decreases in C34 IC₅₀, a gp41 HR2 peptide analog that binds to the helical region 1 (HR1) coiled coil in a fusion intermediate conformation of gp41 [6, 40, 41], were observed for the revertants in relation to the WT (P<0.05, WL/KH and WL/KH/DE versus WT; 2-tailed t test, unequal variances). Notably, WL/KH and WL/KH/DE exhibited markedly greater sensitivity to neutralization by the MPER-specific brNAbs 2F5 and 4E10 when compared to the WT. These data are consistent with structural changes in the gp120-gp41 complex that increase the availability of neutralization targets in the MPER of gp41.

### DISCUSSION

The forced evolution of WL/KD mutant viruses with severely disrupted gp120-gp41 association led to the emergence of replication-competent revertants containing a D601H pseudoreversion in the DSR plus D674E or D674N 2^{nd} site mutations in the MPER. In the case of WLKD-2 clones the ΔT394-W395 deletion in V4 was also observed. The WL/KH and WL/KH/DE viruses exhibited greater sensitivity to the brNAbs, 2F5 and 4E10, indicating that the restoration of function was associated with structural changes in Env that increase the accessibility of neutralization epitopes within the MPER. Our data reveal a functional linkage between the DSR and MPER of gp41 and a novel approach for improving the accessibility of conserved neutralization epitopes within the MPER in a virion context.

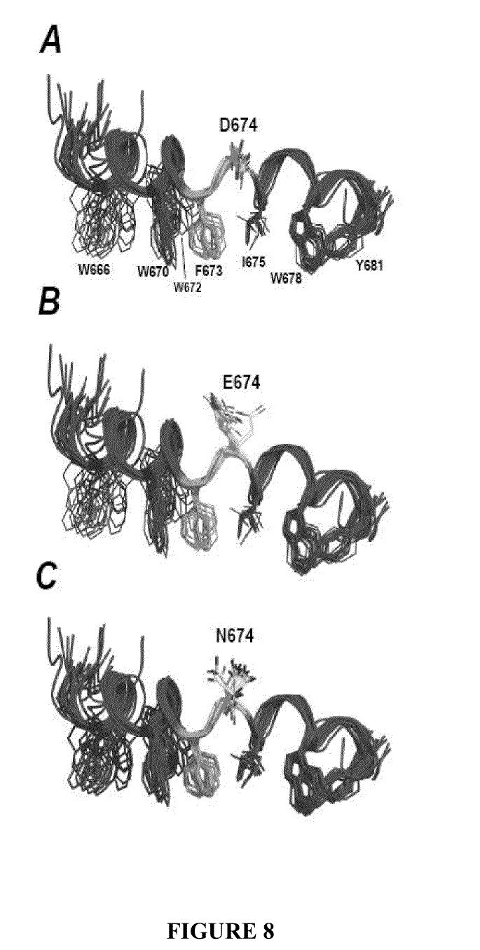

The severe shedding phenotype of WL/KD is likely to have resulted from the combined effects of decreased hydrophobic side chain bulk and an additional negative charge in the contact site that destabilizes gp120-gp41 association. Phenotypic analysis of the revertant genotypes indicated that D601H is a key evolutionary step that partially restores gp120-gp41 association. Histidine at 601 introduces animidazole moiety into the association site, which would partially compensate for the loss of the indole ring of Trp-596 and removes the negative charge contributed by Asp-601. Interestingly, Leu-596 was maintained in both long-term cultures, indicating that the smaller hydrophobic sidechain is preferred at this position when His is present at 601. The combination of His-601 with D674E led to improved single-cycle and multi-cycle cell-free virus-initiated infectivity without detectable further improvement in gp120-gp41 association. The MPER mutation therefore appears to act at the level of virus entry. The modelling of this change into the 3D structure of an MPER peptide determined by NMR (PDB entry, 2PV6 [25]) suggests that the compensatory nature of D674E is related to MPER flexibility. In membranes, the MPER comprises an N-terminal helix connected to a C-terminal helix via a hinge composed of Phe-673, which is buried in the lipid phase, and a polar residue at position 674, which is solvent-exposed (FIG. 8) [25]. The modelling into this structure of Asp-674 suggests that its sidechain will hydrogen bond via Oδ1 with the backbone amides of Asn-674 and Ile-675 in 17 of 17 conformers (FIG. 8A), thereby conferring rigidity to the interhelical hinge. By contrast, an additional methyl group within the Glu-674 sidechain moves the terminal carboxylate out of hydrogen bonding range in 15/17 conformers, consistent with hinge flexibility (FIG. 8B). An intermediate situation is predicted for the alternative D674N site MPER mutation, where sidechain-backbone hydrogen bonds occur in 10 of 17 conformers (FIG. 8C). Interestingly, the presence of Gly-674 in WL/KH-containing clones led to complete blockade of viral infectivity. This observation suggests that a high degree of backbone rotational flexibility within the MPER hinge due to the absence of a side chain in the case of Gly is deleterious to WL/KH function.

In contrast to the data obtained with cell-free virions, replication levels at (WL/KH, WL/KH/DN, ΔTW/WL/KH/DN) or better (WL/KH/DE) than those of WT were achieved by the revertants when infections were initiated with VSV G pseudotyped viruses. In this latter system, the initial infection rounds will be largely mediated by the highly fusogenic VSV G present in the viral envelope thereby normalizing WT and mutant virus infectivity, while subsequent infection rounds will be mediated by gp120-gp41 present on virions transmitted directly from cell to cell via virological synapses, in addition to nascent cell-free virions. Direct cell-cell viral transmission has been calculated to be at least 8-times more efficient than cell-free viral spread and is believed to be due to higher effective multiplicity of infection and virus viability within virological synapses [35]. It may be that in the case of cell-free virions, which encounter receptors following solution-phase diffusion, gp120 is shed from unstable WL/KH, WL/KH/DE and WL/KH/DN gp120-gp41 complexes during the lag time between budding and attachment, thereby decreasing infectivity over time. By contrast, in directed viral transmission across virological synapses, budding, receptor binding, virion maturation and entry appear to be closely linked and to occur over short timeframes [34, 42, 43], which may limit the loss of gp120 from virions prior to receptor encounter. Dale et al. [42] recently reported that in cell-cell viral transmission, receptor attachment is mediated by immature virions, and Env activation for fusion occurs later following viral maturation in the endosome. This contrasts cell-free virus infection where receptor binding is largely mediated by mature virions. It may be that the reverting mutations act optimally in the context of a virion gp120-gp41 complex maintained in an inactive form through interactions between the gp41 cytoplasmic domain and immature Gag [37, 39], prior to receptor engagement and activation for fusion. Overall, these data suggest that the cell-cell mode of viral spread plays the key role in the mechanism of reversion. This idea is consistent with the finding that the WL/KH/DN and ΔTW/WL/KH/DN env genotypes coexist with WL/KH in the WLKD-2 culture even though the former exhibited lower cell-free virus infectivity. It is interesting that the boosts to infectivity associated with the addition of D674E to W596L/K601H did not correlate with enhanced cell-cell fusion activity, where the cell-surface expressed glycoproteins function independently of other virion components. These data are consistent with a Leu-596-His-601-Glu-674 functional interaction that is dependent on virion assembly and the structural constraints imposed on the Envectodomain, including the MPER, by Gag-gp41 cytoplasmic tail interactions [36-39].

Early mutational studies indicated that the DSR is associated with the C1 and C5 regions of gp120 [11-13, 33], which project from the base of gp120 [17] (FIG. 1A). The modelling of these projections in a trimeric context suggests that they will form a layer that encases the DSR [16], thereby occluding it from antibody recognition [44, 45]. On the other hand, the MPER is believed to occupy a spatially distinct location at the base of the gp41 trimer, partially embedded in the envelope and in certain HIV-1 strains, available for antibody binding [44-46]. The MPER-DSR functional linkage is therefore likely to operate via an allosteric mechanism that involves other structural elements of the ectodomain. The HR1 of gp41 is implicated as one such element by the finding that T569A (HR1) and I675V (MPER)polymorphisms synergise in conferring a neutralization sensitive Env conformation [47]. In turn, HR1 has been functionally linked to the receptor and coreceptor binding sites of gp120 and HR2 by fusion inhibitor and neutralizing antibody-driven viral evolution studies [48, 49]. While we did not observe significant changes in sensitivity to the CD4-binding site-directed brNAb b12 or to maraviroc, suggesting that alterations to the receptor and coreceptor binding sites did not contribute to the mechanism of reversion, a small (0.5 log₁₀) but significant increase in C34 sensitivity was observed for WL/KH and WL/KH/DE viruses. Subtle alterations to the function of HR1 may therefore accompany the WL/KH and WL/KH/DE changes.

The WL/KH (and WL/KH/DE) mutations were associated with sensitivity to the 2F5 and 4E10 MPER-directed brNAbs, indicating a structural change in Env that increases MPER accessibility. Two neutralization mechanisms have been proposed for 2F5 and 4E10: i) direct interaction with the MPER in neutralization-sensitive viral strains; ii) Env-receptor interaction-triggered MPER accessibility to brNAb in resistant strains [46]. We have found that the macrophage-adapted AD8 strain is relatively resistant to these brNAb suggesting that the MPER is sterically occluded in the WT viral AD8 Env complex and the WL/KH gp120-gp41 association site mutation leads to a more open Env structure, enabling better epitope access for 2F5 and 4E10. Alternatively or additionally, WL/KH may be associated with structural change in the MPER itself, within creased MPER flexibility and/or altered membrane interactions facilitating paratope-mediated extraction of the 2F5 and 4E10 epitopes from the envelope. We previously reported that the contributions of Trp-596 and Lys-601 to gp120-gp41 association and membrane fusion are influenced by sequence changes in V1, V2, and V3 [33], which are predominantly associated with the evolution of neutralization resistance [50-56], as well as coreceptor preference and cellular tropism [57-60]. Thus the gp120-gp41 association site appears structurally and functionally adaptable, perhaps to maintain glycoprotein function during gp120-gp41 evolution. The observation here of functional crosstalk between the DSR and MPER implies that the structural adaptation of the gp120-gp41 synapse in order to cope with the evolution of other glycoprotein domains is also linked to changes in MPER structure that alters the ability of conserved neutralization epitopes therein to be bound by antibody.

MPER-specific brNAbs are of particular interest to the HIV-1 vaccine field due to the conserved nature of their epitopes and their neutralization breadth [20, 61-64]. Biophysical and structural studies have indicated that membrane-anchored MPER conformations are optimally bound by 2F5 and 4E10-like brNAbs [65], however the goal of developing a vaccine that presents the MPER in a lipid environment and produces high-titre 2F5- and 4E10-like brNAbs has not yet been realized [32, 66-69]. The data presented here indicate that changes to the gp120-gp41 association site can increase the availability of the 2F5 and 4E10 epitopes in virus and point to a new approach for improving the accessibility of MPER epitopes in virion-based immunogens.

### Example 2

### Materials and Methods

### Env Expression Vectors and Proviral Clones

The preparation of the cytomegalovirus promoter-driven HIV-1_{AD8} Env expression vector, pCDNA3.1-AD8env, is described elsewhere [28]. pΔKAD8env was derived by religation of the end-filled HindIII and EcoRI sites of pCDNA3.1-AD8env. In vitro mutagenesis of the gp41 region was carried out by overlap extension PCR. Mutants of the pAD8 infectious clone (obtained from K. Peden [89] were prepared by transferring the EcoRI-BspMI env-containing fragment from pCDNA3.1-AD8env vectors into pAD8. Bacteriophage T7 promoter-driven gp120 expression vectors, based on pTM.1 [90], were generated by ligating PCR-amplified HIV-1_{AD8} gp120 fragments into the NdeI and StuI sites of pTMenv.2 [91] to give pTM-AD8gp120.

### Infection of PBMCs

PBMC infections were conducted as described previously [92]. Briefly, PBMCs isolated from buffy packs (Red Cross Blood Bank, Melbourne) were stimulated with phytohemagglutinin (10 μg/ml; Murex Diagnostics) for 3 days in RPMI 1640 medium containing 10% fetal calf serum and interleukin-2 (10 units/ml; Boehringer-Mannheim). Virus stocks were prepared by transfecting 293T cell monolayers with pAD8 infectious clones using Fugene 6 (Roche). Virus-containing transfection supernatants were normalized according to reverse transcriptase (RT) activity, and then used to infect 10⁵ PBMCs in a 96-well tissue culture plate (eight 10-fold serial dilutions of each virus were tested in triplicate). The supernatants were assayed for RT activity at various time points.

### Sequential Passage of Cell-Free K601D Virus in PBMC

Phytohemagglutinin-stimulated PBMCs were infected with equivalent amounts of wild type (WT) and K601D-mutated HIV-1_{AD8} (according to RT activity) in parallel and maintained in culture for 10 days. Cell-free culture supernatants were filtered (0.45 μm pore size) and normalized according to RT activity prior to the next passage (5 passages in total). Genomic DNA was extracted from infected PBMCs using Qiagen DNeasy. The viral DNA fragment encompassed by nucleotides 5954-9096 (HXB2R numbering convention) was PCR-amplified using Expand HiFi (Roche) and the primers, 5′-GGCTTAGGCATCTCCTATGGCAGGAAGAA, SEQ ID NO:1, (Env1A) and 5′-TAGCCCTTCCAGTCCCCCCTTTTCTTTTA, SEQ ID NO:2, (Env1M) [93]. The amplified sequences were ligated into pGEM-T or pΔKAD8env (KpnI-XbaI) and the entire env open reading frame sequenced using ABI BigDye terminator 3.1.

### Western Blotting

Lysates of Env-expressing 293T cells or virions pelleted from pAD8-transfected 293T cell supernatants were subjected to SDS-PAGE under reducing conditions, transferred to nitrocellulose and then probed with mAb C8 to gp41 (from G. Lewis [94], DV-012 to gp120 (from M. Phelan [95,96], or mAb 183 to CA (from B. Chesebro and K. Wehrly [97,98](AIDS Research and Reference Reagent Program, NIAID) as described [28].

### Luciferase Reporter Assay of Cell-Cell Fusion

Cell-cell fusion assays were conducted as described [76]. Briefly, 293T effector cells were cotransfected with pCDNA3.1-AD8env or pΔKAD8env and pCAG-T7 [99] plasmids, while BHK21 target cells were cotransfected with pT4luc [27] and pc.CCR5 (AIDS Research and Reference Reagent Program from N. Landau [100] or a panel of CCR5 mutants in the pcDNA3 expression vector (kind gifts of J. S. Sodroski and R. W. Doms [101,102]). The Y14N mutation was introduced to pc.CCR5 using the Quikchange II XL kit (Stratagene). At 24 h posttransfection, targets and effectors were cocultured in triplicate in a 96-well plate (18 h, 37° C.) and then assayed for luciferase activity (Promega SteadyGlo, Madison, Wis.). The sensitivities of WT and mutant Env proteins to the fusion inhibitor peptide C34 (WMEWDREINNYTSLIHSLIEESQNQQEKNEQELL, SEQ ID NO:3; Mimotopes, Australia [57]) were determined by coculturing effector and target cells in the presence of serially diluted C34. The sensitivities of WT and mutant Env proteins to sCD4 (NIH AIDS Research and Reference Reagent Program) were determined by incubating the Env-expressing 293T cells with a dilution series of sCD4 for 3.5 h followed by coculturing the effector and target cells in the presence of sCD4 for 8 h.

### Single Cycle Infectivity Assays

Single cycle infectivity assays were conducted as described [76]. Briefly, Env-pseudotyped luciferase reporter viruses were produced by cotransfecting 293T cells with pCDNA3.1-AD8env or pΔKAD8env vectors plus the luciferase reporter virus vector, pNL4.3.Luc.R⁻E⁻ (AIDS Research and Reference Reagent Program, N. Landau [103]), using Fugene 6. The infectivity of pseudotyped viruses was determined in U87.CD4.CCR5 cells (AIDS Research and Reference Reagent Program, H. Deng and D. Littman[104]).

### Biosynthetic Labelling and Immunoprecipitation

293T cells were transfected with pCDNA3.1-AD8env or pΔKAD8env vectors. At 24-h posttransfection, the cells were incubated for 30 min in cysteine and methionine-deficient medium (MP Biomedicals, Seven Hills, NSW, Australia), and then labelled for 45 min with 150 μCi Tran-³⁵S-label (MP Biomedicals). The cells were then washed and chased in complete medium for 5-6 h prior to lysis. Cell lysates and clarified culture supernatants were immunoprecipitated with IgG14 or HIVIG and protein G Sepharose and subjected to SDS-PAGE in the presence of β-mercaptoethanol [28]. The labelled proteins were visualized by scanning in a Fuji phosphorimager. Quantitation of bands was performed using Image Gauge (FUJIFILM) software.

gp120-sCD4 Binding Assay

293T cells were cotransfected with pTM-AD8gp120 and pCAG-T7 vectors using Fugene 6. At 24-h posttransfection, the cells were incubated for 30 min in cysteine and methionine-deficient medium, labelled for 45 min with 150 μCi Tran-³⁵S-label, and then washed and chased in complete medium for 6 h. The clarified culture supernatants were adjusted to 0.6 M KCl, 1 mM EDTA and 1% w/v Triton-X100 and the gp120 content quantified following immunoprecipitation with IgG14 and protein G Sepharose, reducing SDS-PAGE and scanning in a Fuji phosphorimager. Equivalent amounts of WT and mutant gp120 proteins were incubated with serial dilutions of sCD4 (1 h, room temperature) and then incubated with monoclonal antibody (mAb) OKT4 and BSA-Sepharose on a vibrating platform (30 min, room temperature). After pelleting the BSA-Sepharose, protein complexes were coprecipitated using protein G-Sepharose and gp120 quantified following reducing SDS-PAGE and scanning in a Fuji phosphorimager.

### Neutralization Assay

Neutralization assays were conducted using a modification of the method of Dhillon et al. [105]. Briefly, a solution of Env-pseudotyped luciferase reporter viruses determined previously to give 5×10⁵ relative light units (RLU) following infection of U87.CD4.CCR5 cells was mixed with an equal volume of serially diluted IgG and incubated for 1 h at 37° C. One hundred microliters of the virus-IgG mixtures was then added to U87.CD4.CCR5 cells (10⁴ cells per well of a 96-well tissue culture plate, 100 microlitres) and incubated for 2 days prior to lysis and assay for luciferase activity (Promega, Madison, Wis.). Neutralizing activities for antibody samples were measured in triplicate and reported as the average percent luciferase activity. Purified IgG of monoclonal NAbs 2F5, 2G12 and IgGb12 were obtained from Polymun Scientific (Austria), PG16 was obtained from the International AIDS Vaccine Initiative, while HIVIG was obtained from F. Prince through the NIAID AIDS Research and Reference Reagent Program.

### Molecular Modelling

A homology-based model of HIV-1_{AD8} V1V2 was generated from PDB entry 3U4E [7] using the Modeller algorithm [106] within Discovery Studio, version 3.0 (Accelrys). Five independent models were generated from iterative cycles of conjugate-gradient energy minimization against spatial constraints derived from the template crystal structure. The model with the lowest energy (probability density function) was glycosylated in silico with oligomannose side chains using the glycosciences.de server [107,108].

### Results

### Phenotype and Long-Term Culture of the HIV-1_{AD8}-K601D DSR Mutant

The gp120-gp41 association defective DSR mutant HIV-1_{AD8}-K601D was subjected to serial PBMC passage in order to select suppressor mutations. It was reasoned that the locations of suppressors would point to structural elements that are functionally linked to the DSR, thereby shedding light on its role in Env function. It was first confirmed that the K601D mutation promoted the shedding of gp120 into the culture supernatant of 293T cells transfected with pcDNA3.1-AD8env, as determined by radio immunoprecipitation (FIG. 9C). Consistent with this finding, K601D-mutated HIV-1_{AD8} virus particles were largely devoid of gp120 (FIG. 9D). The presence of gp160 in the pelleted virion preparations was noted. It likely represents one of a number of non-native Env forms that have been observed in virions and have been implicated as immune decoys [45,46,47,48,49,50]. The gp120 shedding defect did not appear to be associated with a precursor processing defect as Western blotting with the gp41-directed C8 monoclonal antibody, indicated that similar amounts of gp41 were derived from gp160 for both WT and K601D (FIG. 9E). Cell-to-cell fusion function was blocked by K601D at sub-saturating Env conditions (FIG. 9F), while HIV-1_{AD8} viral replication over 10 days in phytohemagglutinin-stimulated peripheral blood mononuclear cells (PBMCs) from independent donors was severely attenuated by the mutation (FIG. 9G). The restoration of HIV-1_{AD8}-K601D replication competence was observed with sequential passage of cell-free virus in independent PHA-stimulated PBMC cultures (FIG. 9G, cultures P0 and P2).

### Genotypes of HIV-1_{AD8}-K601D Revertants

In order to identify suppressors of K601D, the env region was PCR-amplified from genomic DNA isolated from infected cells at days 20, 30, 40 and 50 for culture P0, and days 30 and 50 for culture P2. The PCR products were cloned into the pΔKAD8env expression vector for DNA sequencing. The K601D mutation was retained in P0 clones isolated at days 20 and 30 (FIG. 10A), when replication was not observed in the mutant virus culture (FIG. 9G, P0). The emergence of replication-competent virus at day 40 in the P0 culture correlated with the appearance of L494I in C5 of gp120 (6/6 clones), together with either a D601N pseudoreversion (3/6 clones), and/or T138N, which abolishes a conserved PNGS at position 136 (N¹³⁶VT) within V1 of gp120 (3/6 clones). By day 50, when mutant viral replication approached that of WT, the T138N/L494I/K601N triple mutation was present in 42% of clones. A single clone (P0.D50.10) contained S144N, which ablates the nearby PNGS at N¹⁴²SS.

In an independent PBMC long-term culture (P2), the replication competence of the mutant virus approached that of WT by the 3rd passage (FIG. 9G, P2). Deletion of residues N¹³⁹INN (ΔN¹³⁹INN) from V1, that results in the loss of overlapping PNGSs at positions 141 and 142 (N¹⁴¹N¹⁴²SS), was observed in 3/14 P2 day-30 clones, while 2/14 possessed mutations at Thr¹³⁸; K601N was also observed in 50% of P2-day 30 clones (FIG. 10B). The dominant genotypes in P2 day-50 clones were ΔN¹³⁹INN together with K601N, and K601N alone. A 2-residue N¹⁴²S¹⁴³ deletion was observed in 1 day-50 clone (P2.D50.141), which also ablates the overlapping PNGSs at 141 and 142. Interestingly, the L494I mutation was not observed in P2 clones. These data indicate that PNGSs in V1 are functionally linked to the gp120-gp41 association synapse.

### Functional Linkages Between the DSR, PNGSs in V1, and Leu⁴⁹⁴ in C5

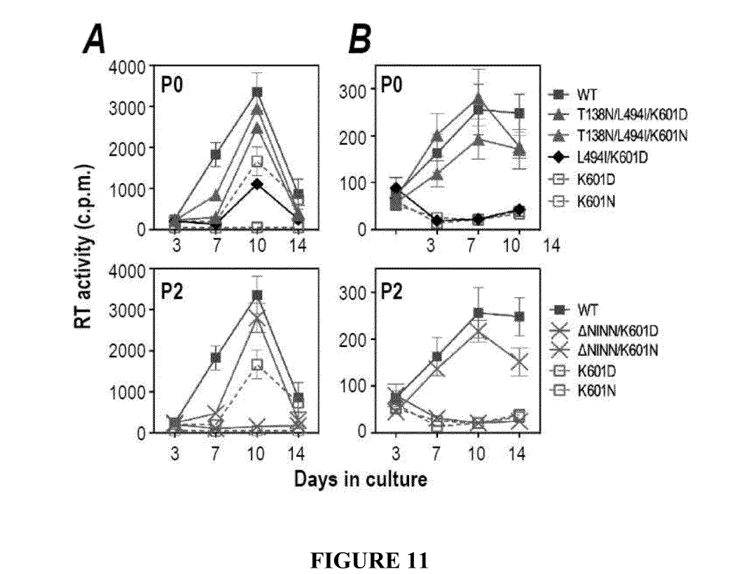

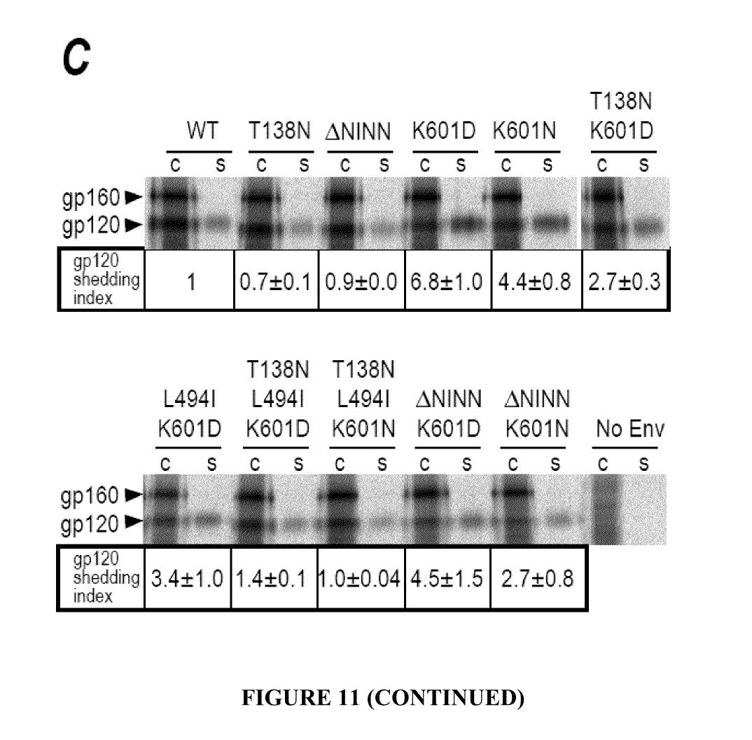

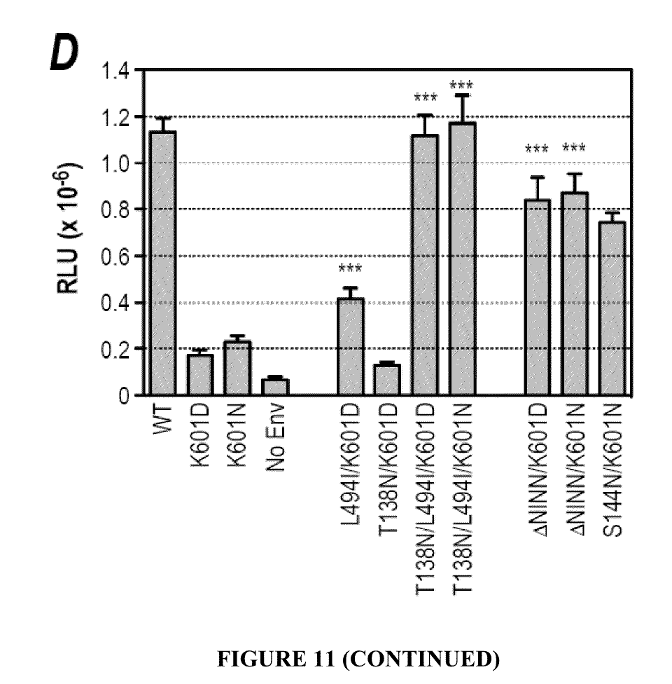

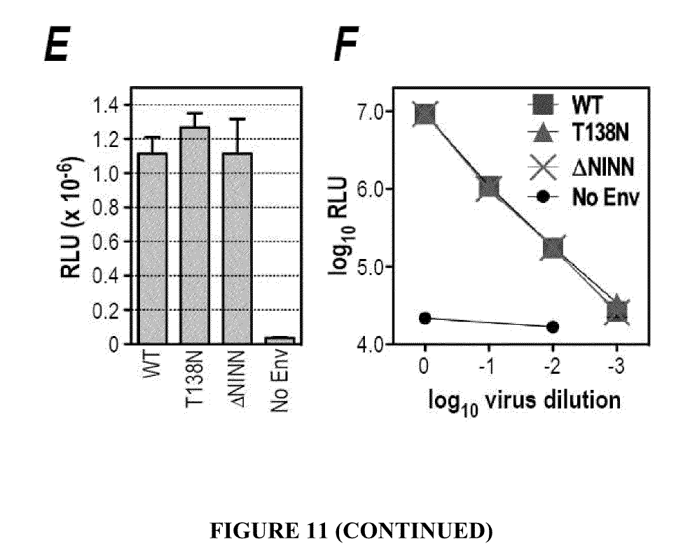

To determine how replication was restored to the K601D virus in the long-term PBMC cultures, we reconstructed the dominant P0 and P2 genotypes in the context of the pAD8 proviral clone and examined viral replication in two independent PBMC donors. Both P0 mutations, L494I within C5 and K601N within the DSR, partially restored replication in the PBMCs of one donor but not in the second, apparently less permissive donor (FIGS. 11A and B; P0 panels). The addition of T138N, which ablates the PNGS at Asn¹³⁶ within V1, led to near-WT replication kinetics for T138N/L494I/K601D and T138N/L494I/K601N genotypes in PBMCs from both donors. These data suggest that T138N and L494I act synergistically to suppress K601D. The analysis of P2 genotypes in PBMC cultures indicated that the N¹³⁹INN deletion in V1 was not sufficient to suppress K601D, but its combination with K601N led to near-WT levels of viral replication (FIG. 11A, B; P2 panels).

The pΔKAD8env expression vector was next employed to further dissect the functional linkages between position 601 within the DSR, the Asn¹³⁶ and Asn^{141/142}PNGS mutations in V1 and the L494I mutation in C5 in gp120-gp41 association and cell-cell fusion assays. The results presented in FIG. 11C indicate that K601D and K601N DSR mutations promote shedding of gp120 into culture supernatants of transfected cells by ˜6.8- and 4.4-fold with respect to WT, respectively, while cell-cell fusion activity was reduced by ˜85-90% (FIG. 11D). These data indicate that the K601D and K601N DSR mutations inhibit cell-cell fusion function by destabilizing the gp120-gp41 complex. However, the effect of K601N on Env function in the context of virus replication appears to be donor dependent (see FIGS. 11A and B). It may be that lower receptor numbers and/or alternate coreceptor post-translational modifications on target cells, or differences in target cell population numbers [51,52,53,54] in the case of the latter donor's PBMCs do not enable the fusion-activation threshold to be reached for K601N Env. A general correlation between the restoration of cell-cell fusion activity and gp120-gp41 association was observed for the P0 combination mutants. Thus L494I/K601D exhibited ˜50% of WT cell-cell fusion activity with improved gp120-gp41 association, whereas T138N/L494I/K601D and T138N/L494I/K601N were functionally similar to the WT. The phenotype of T138N/K601D was an outlier as gp120-gp41 association was improved by ˜2-fold with respect to K601D without a restorative effect on Env fusion function. These data indicate that T138N and L494I act cooperatively to restore gp120-gp41 association to K601D with concomitant restoration of membrane fusion function and viral replication competence. T138N-containing gp120 molecules migrated to lower molecular weight positions with respect to WT in reducing SDS-PAGE, consistent with loss of the glycan at Asn¹³⁶ (FIG. 11C).

The ΔN¹³⁹INN/K601D and ΔN¹³⁹INN/K601N P2 clones exhibited 74-77% of WT fusion activity (P<0.05) with only marginal restoration of gp120-gp41 association, even though only the latter clone was competent for replication in PBMCs (FIGS. 11A-D). The suppression of the K601N fusion and replication phenotype by ΔN¹³⁹INN is therefore not dependent on the full restoration of gp120-gp41 association nor on the L494I C5 mutation.gp120 molecules with the ΔN¹³⁹INN mutation migrated faster in SDS-PAGE than non-ΔN¹³⁹INN-containing Envs, again consistent with the loss of glycan (FIG. 11C). The ΔN¹³⁹INN mutation disrupts the overlapping N-linked glycosylation sequons: Asn¹⁴¹-Asn¹⁴²-Ser-Ser. Such overlapping N-linked glycosylation sequons are observed in V1 of a subset of HIV-1 strains, although the position varies. Combining S144N (a PNGS mutation observed in clone P0.D50.10) with K601N resulted in almost identical fusion activity to ΔN¹³⁹INN/K601N (FIG. 11D), suggesting that loss of the Asn¹⁴² glycan can substitute for the N¹³⁹INN deletion.

It was next asked whether the V1 PNGS mutations restored function via a functional link to Asp or Asn at position 601 in the DSR or whether a generalized enhancement in Env function could explain the restored phenotypes. FIGS. 11E and F indicate that T138N and ΔN¹³⁹INN do not increase the cell-cell fusion activity of surface expressed Env or the infectivity of Env-pseudotyped luciferase reporter virus when introduced to the WT background. It was also noted that the L494I mutation in a WT background did not substantially increase cell-cell fusion or gp120-gp41 association (data not shown). These data are consistent with specific functional crosstalk between the V1 glycans, the Asp⁶⁰¹ or Asn⁶⁰¹ in the DSR and position 494 in C5.

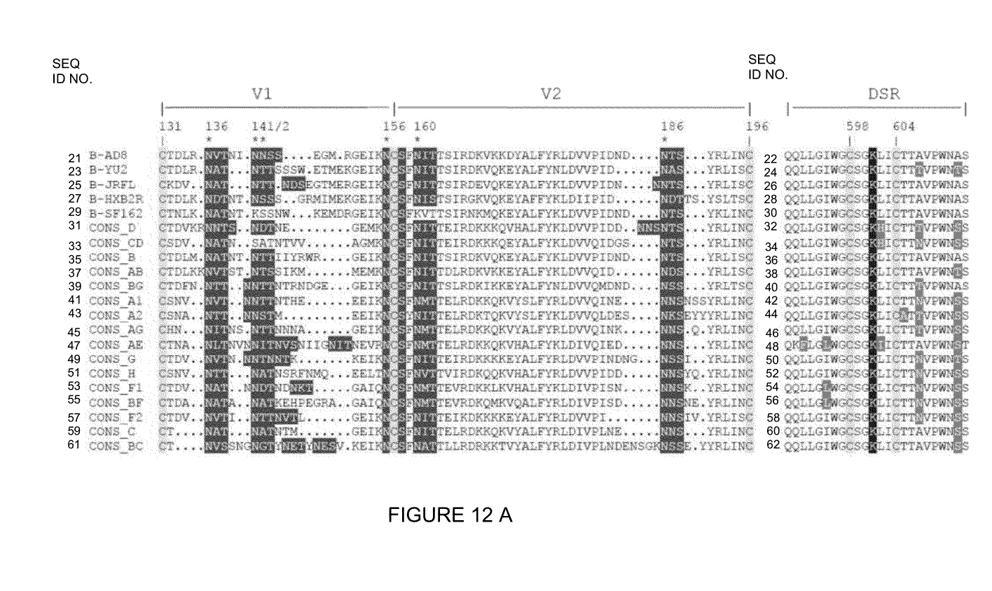

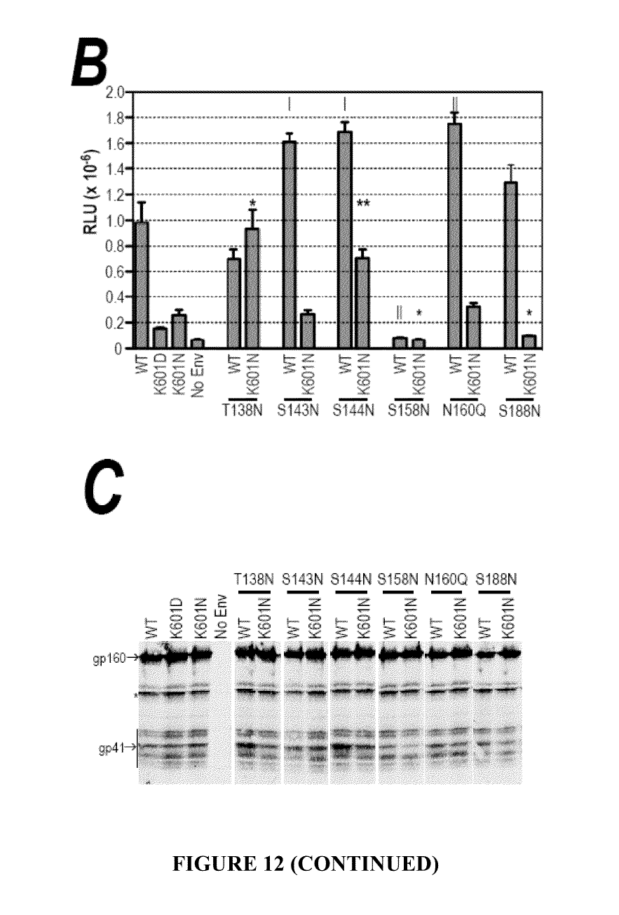

Finally, it was asked if the V1 PNGS mutations at 136 and 142 restored function via a specific link to position 601 in the DSR, or whether deletion of any of the 6 PNGSs in V1V2 of the AD8 strain (FIG. 12A) could restore functional defects related to a DSR mutation. To this end, T138N, S143N, S144N, S158N, N160Q and S188N mutations were introduced to the WT and K601N pΔKADenv vectors and determined their effects on cell-cell fusion, glycoprotein processing and gp120-gp41 association. FIG. 12B indicates that in a WT Env context, T138N and S188N did not affect cell-cell fusion, S143N, S144N and N160Q led to small but significant functional enhancements, whereas S158N blocked fusion completely. Even though, most of the V1V2 glycan mutants were fusogenic on a WT background, only T138N and S144N restored function to K601N. A Western blot of ΔKADenv-transfected 293T cell lysates with the gp41-specific mAb, C8, confirmed that the mutant glycoproteins were expressed and processed to gp41 at levels that were close to the WT (FIG. 12C). Pulse-chase biosynthetic labelling followed by radio immunoprecipitation with pooled IgG derived from HIV-1-infected individuals (HIVIG) indicated WT-like gp120-gp41 association for T138N, S143N, S144N, N160Q and S188N and a shedding phenotype for the fusion-defective S158N (FIG. 12D). Near-WT levels of gp120-gp41 association were observed for the fusion-competent T138N/K601N mutant, whereas S144N/K601N, which is also fusion competent, exhibited a mild gp120 shedding phenotype. By contrast, improvements in gp120-gp41 association did not follow the combination of K601N with S143N, S158N, N160Q or S188N, consistent with their lack of fusion activity. Overall, these data are consistent with a specific functional linkage between the DSR and the Asn¹³⁶ and Asn¹⁴² glycosylation sites in V1.

### Mechanism of Reversion

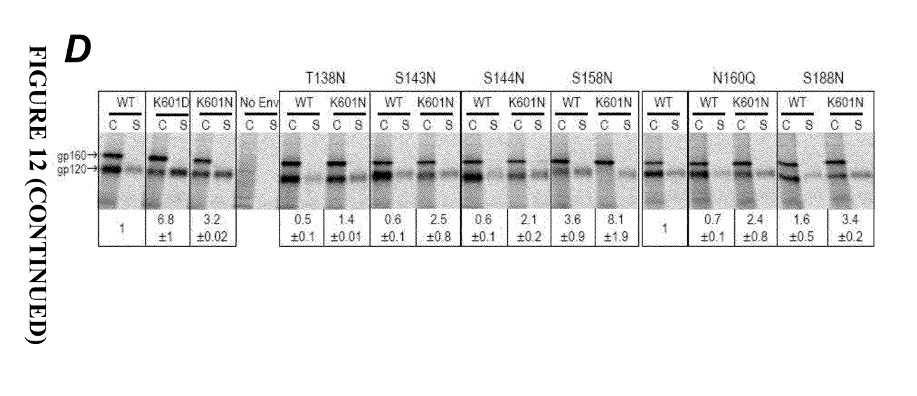

The effects of T138N, L494I and ΔN¹³⁹INN on CD4 binding ability, sensitivity to sCD4 inhibition, CCR5 utilization and sensitivity to the C34 fusion HR2-based inhibitor peptide were examined in order to further elucidate the mechanism whereby function was restored in the context of a mutated DSR. CD4 binding curves were generated by incubating a constant amount of biosynthetically labelled WT and mutated gp120 with serial dilutions of sCD4, coimmunoprecipitation of gp120-sCD4 complexes with mAb OKT4, followed by SDS-PAGE and densitometry of gp120 bands. Similar CD4 binding curves were observed for WT, T138N/L494I and ΔN¹³⁹INN gp120 mutants (FIG. 13A) indicating that the glycosylation site mutations in V1 and L494I in C5 did not alter the CD4-binding ability of gp120. The sensitivity of T138N/L494I/K601N-, ΔN¹³⁹INN/K601N- and WT-Env-mediated fusion to inhibition with sCD4 was next tested. The Env-expressing 293T cells were incubated with a sCD4 dilution series for 3.5 h prior to an 8-h coculture with CD4-plus-CCR5-expressing BHK21 targets harboring a luciferase reporter. FIG. 13B indicates that WT and T138N/L494I/K601N have almost identical sensitivities to sCD4 IC₅₀s of 60 μg/ml, which are comparable to previously published values for both T cell-line adapted and primary HIV-1 Envs [55]. The ΔN¹³⁹INN/K601N inhibition curve, however, was shifted by ˜1 log₂ to the left, indicating that this Env is slightly more resistant to sCD4 even though gp120 containing the ΔN¹³⁹INN mutation had similar sCD4 binding characteristics to WT and T138N/L494I.

The abilities of T138N/L494I/K601N, ΔN¹³⁹INN/K601N and WT Envs to utilize a panel of CCR5 coreceptor mutants were next compared in order to determine if alterations to the mode of CCR5 engagement could account for reversion. The results presented in FIG. 13C indicate that the T138N/L494I/K601N, ΔN¹³⁹INN/K601N and WT Envs exhibited almost identical patterns of mutant CCR5 utilization. For example, Q4A and Y14A (N-terminal domain, Nt), H88A (extracellular loop 1, ECL1), K171A, E172A and Q188A (extracellular loop 2, ECL2), and F264A and R274A (extracellular loop 3, ECL3) CCR5 mutants supported fusion with the 3 Env constructs to the same extent as WT CCR5, whereas fusion with Q280A (extracellular loop 3) was decreased to 25-40% of WT CCR5 activity. The comparative abilities of T138N/L494I/K601N, ΔN¹³⁹INN/K601N and WT Envs to mediate cell-cell fusion with the CCR5-Y14N tyrosine sulfation mutant, which exhibits a lower affinity for gp120-gp41 and functions as a HIV-1 coreceptor in a cell surface concentration-dependent manner was determined [56]. The coreceptor activity of WT CCR5 for WT and T138N/L494I/K601N remained at consistently high levels for fusion with BHK21 targets cotransfected with a constant amount of pT4luc vector and a dilution series of pc.CCR5 DNA; the fusion activity of ΔN¹³⁹INN/K601N was slightly diminished across the pc.CCR5 dilution series (FIG. 13D), consistent with the data presented in FIG. 11D. These data are consistent with previous findings indicating that trace amounts of CCR5 are sufficient to mediate efficient fusion and entry by HIV-1 [56]. By contrast, fusion mediated by the 3 Env constructs exhibited a strict dependence on the amount of transfected CCR5-Y14N plasmid without any significant changes in the CCR5-Y14N concentration curves due to the Env mutations being observed. These data indicate that the restoration of function to the mutated DSR by the T138N/L494I or ΔN¹³⁹INN suppressor mutations in gp120 is unlikely to be a result of altered CD4 and CCR5 utilization.

It was considered that an increase in the efficiency of a post receptor-binding event, such as 6-helix bundle formation, could aid in the functional compensation of the DSR mutations. We therefore asked if the mutations in gp120 and K601N led to changes in sensitivity to the HR2 synthetic peptide analogue, C34, which blocks fusion by binding to the coiled coil of HR1 helices in a receptor-triggered prehairpin intermediate conformation of gp41 [17,57]. It was expected that faster 6-helix bundle folding kinetics would correspond to decreased C34 sensitivity. FIG. 13E indicates that T138N/L494I/K601N, ΔN¹³⁹INN/K601N and WT Envs exhibited almost identical C34 fusion inhibition curves with IC₅₀s of ˜100 nM. These data indicate that the T138N/L494I/K601N reversion mechanism is unlikely to be due to changes in gp120-receptor interactions nor post-receptor binding events such as efficiency of 6-helix bundle formation, whereas ΔN¹³⁹INN/K601N may be subtly altered in sCD4-induced changes that inhibit membrane fusion function.

### Modulation of the Gp120-Gp41 Association Site by Adjacent Glycosylation Sites in V1

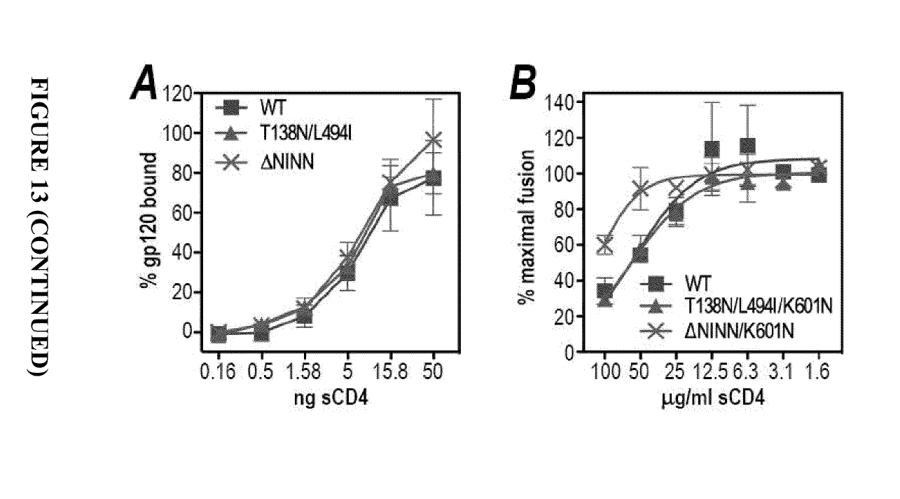

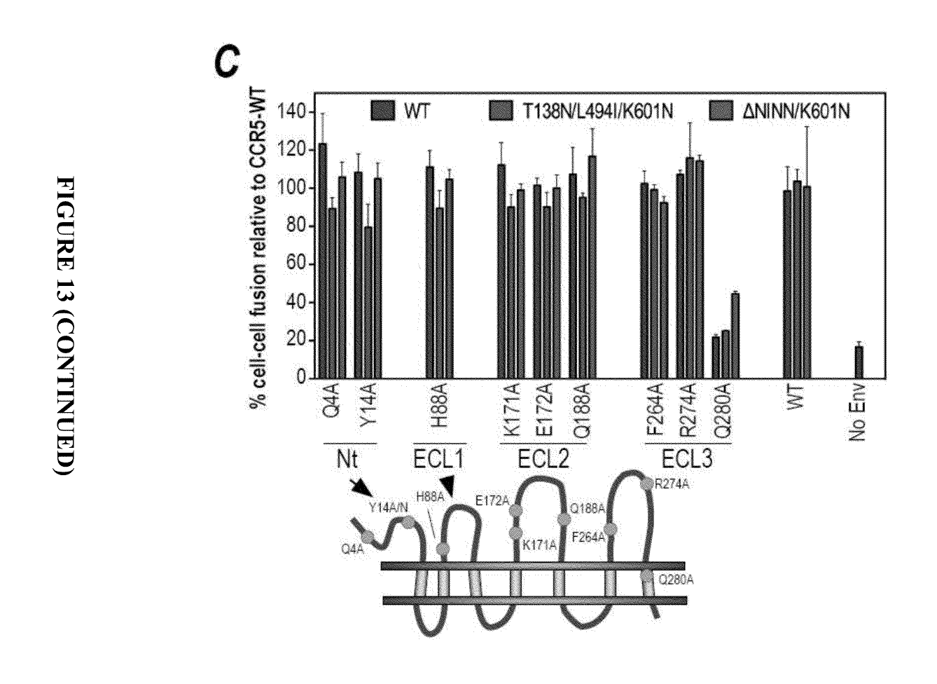

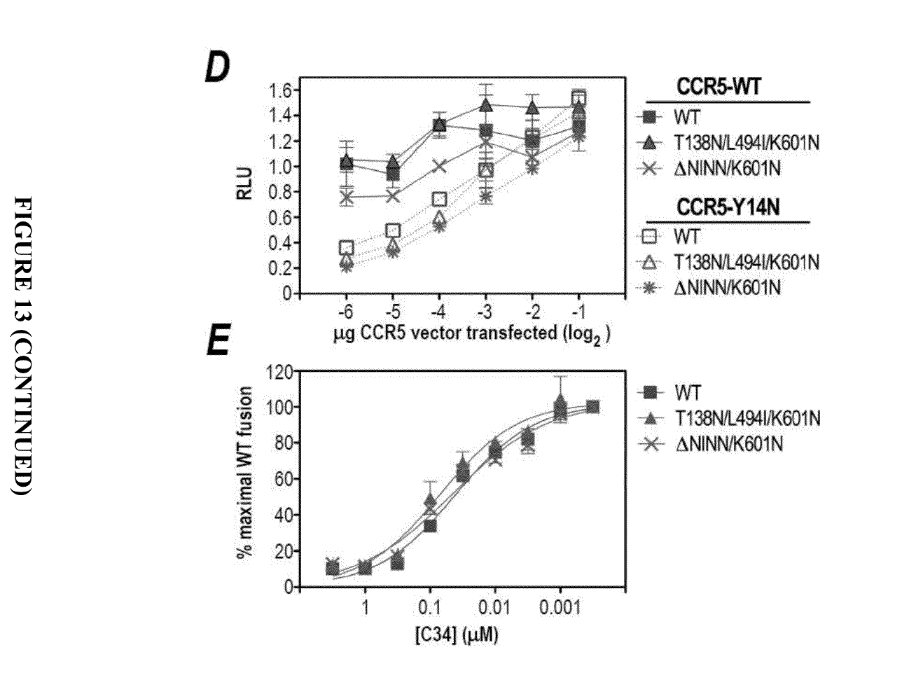

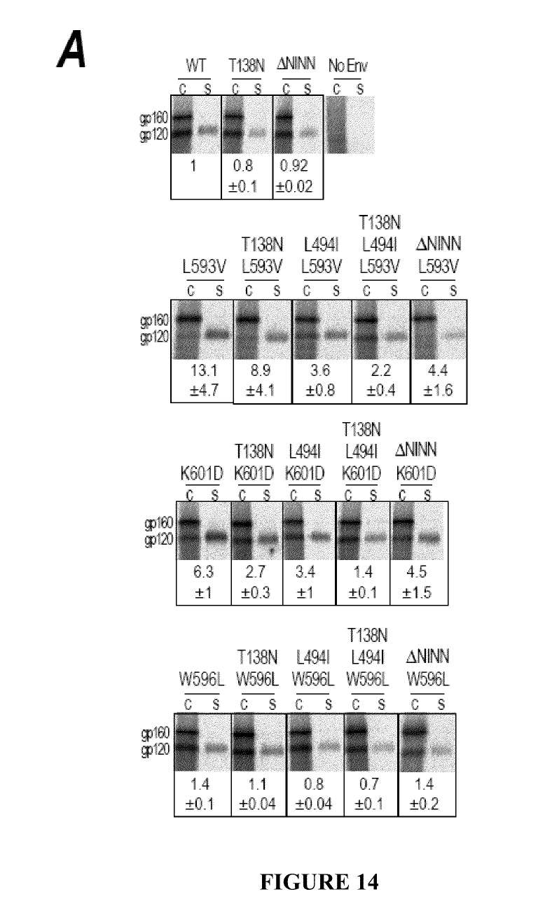

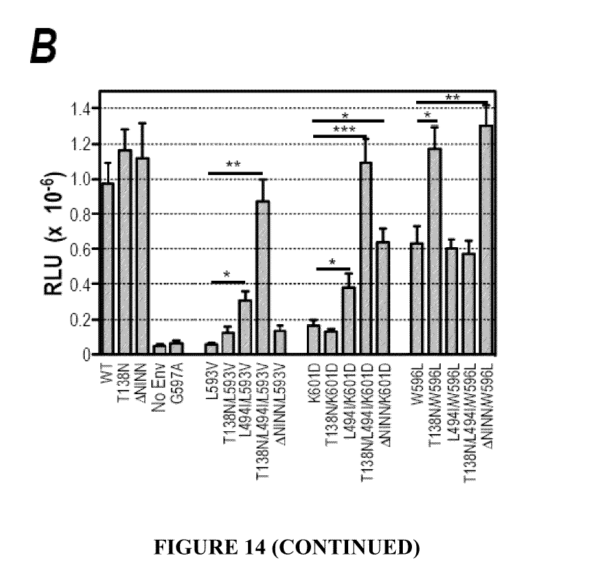

The DSR mediates association with gp120 and may play a role in the activation of gp41 by responding to receptor-induced changes in gp120 [27,28,29,30,31]. To better understand how glycosylation in V1 impacts on gp120-gp41 interactions, the functional effects of T138N, L494I, T138N/L494I and ΔN¹³⁹INN mutations on two other gp120 contact residues within the DSR, Leu⁵⁹³ and Trp⁵⁹⁶, in addition to Lys⁶⁰¹ were assessed [27,28]. While L593V and K601D mutations resulted in decreased gp120-gp41 association and cell-cell fusion function, the W596L mutant exhibited WT levels of gp120-gp41 association but reduced cell-cell fusion by ˜40% at subsaturating Env (P<0.02, 2 sample t-test, unequal variances) (FIG. 14A, B). FIGS. 14A and B indicate that T138N had no effect on cell-cell fusion when combined with L593V or K601D, whereas small improvements in glycoprotein association and fusion function were observed when L494I was added to the DSR mutants; wild type levels of gp120-gp41 association and fusion were attained when both T138N and L494I were combined with L593V or K601D. By contrast, W596L exhibited WT fusion levels in combination with T138N. The N¹³⁹INN deletion did not provide any improvement to L593V fusogenicity, but restored increasing levels of fusion function to K601D and W596L, respectively. These data indicate that T138N in V1 alters the gp120-gp41 association site such that fusion function is less dependent on Trp⁵⁹⁶ and, when L494I is also present, on Leu⁵⁹³ and Lys⁶⁰¹. The N¹³⁹INN deletion renders fusion function less dependent on Trp⁵⁹⁶ and Lys⁶⁰¹.

### Modulation of Neutralization Sensitivity by Adjacent Glycans in V1

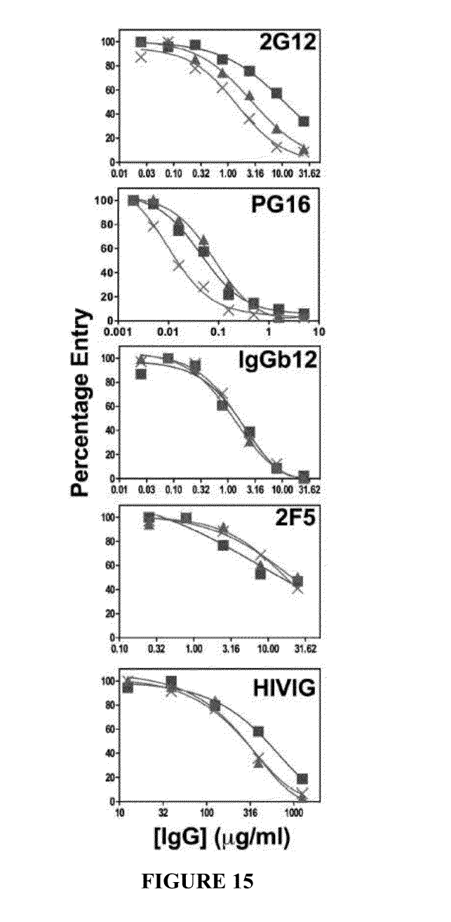

It was therefore asked whether the T138N and ΔN¹³⁹INN mutations were linked to changes in the neutralization sensitivity of Env-pseudotyped reporter viruses. The results (FIG. 15) show step-wise ˜0.5 log₁₀-increases in the neutralization sensitivities of T138N and ΔN¹³⁹INN to the monoclonal NAb 2G12, which is directed to a glycan cluster involving Asn²⁹⁵, Asn³³² Asn³³⁹, Asn³⁸⁶ and Asn³⁹² on the outer face of gp120 [58,59,60,61,62,63]). The 2G12 IC₅₀s for WT, T138N and ΔN¹³⁹INN were 13, 4 and 1.5 μg/ml, respectively. In the case of PG16, which recognizes an epitope in V1V2 involving the Asn¹⁵⁶ and Asn¹⁶⁰ oligomannose glycans [7,64,65], neutralization was enhanced for ΔN¹³⁹INN only (IC₅₀=0.075 and 0.015; IC₉₀=1.6 and 0.16 μg/ml, respectively, for WT and ΔN¹³⁹INN). By contrast, neutralization by IgGb12 (CD4 binding site [66,67]) and 2F5 (membrane proximal external region of gp41 [68,69,70]) was not affected by the V1 mutations. Pooled IgG from HIV-1 infected individuals (HIVIG) was used as a reference reagent. In this case, a reproducible ˜2-fold increase in sensitivity to neutralization by HIVIG was observed with the V1 PNGS mutations (IC₅₀˜550 μg/ml for WT, 280 μg/ml for T138N and ΔN¹³⁹INN) indicating that these glycans are likely to modulate neutralization epitopes recognized by human immune sera. These data indicate that the adjacent V1 glycans shown here to modulate the gp120-gp41 association site are linked to neutralization resistance.

A salient feature of HIV-1 Env is its rapid acquisition of mutations at PNGSs that enable evasion of the adaptive immune response, while maintaining key functions such as gp120-gp41 association, receptor recognition and membrane fusion. Here it was found that glycan changes in V1 that are associated with neutralization sensitivity are linked to a remodelling of the gp120-gp41 association site. It is proposed that this represents a mechanism for the functional adaptation of the highly conserved gp120-gp41 association site to an evolving glycan shield in a setting of neutralizing antibody selection.

The gp120 shedding phenotype of K601D (and K601N) emphasises the importance of the Cys⁵⁹⁸-Ser-Gly-Lys-Leu-Ile-Cys⁶⁰⁴ loop segment of the DSR in gp120-gp41 association. Lys⁶⁰¹ is conserved in HIV-1 isolates (FIG. 9B) but the previously described mild phenotypes of K601Q and K601E [27,28] suggest that the functional role of this residue is not solely dependent on its basic nature. In order to force the evolution of the Env complex and to prevent back reversion (i.e. D601K), it was necessary to mutate both the first and third positions of the Lys codon to create Asp (AAA to GAT). In culture P0, Asp was initially tolerated as a functional replacement of Lys⁶⁰¹ provided that L494I in the gp41-contact site of gp120plusT138Nin V1 were also present. L494I led to slightly improved gp120-gp41 association and fusion function with Asp⁶⁰¹, whereas a WT-like phenotype also required T138N. These data suggest that loss of the Asn¹³⁶ glycan from V1 due to T138N remodels the gp120-gp41 interface such that Asp⁶⁰¹ is better accommodated, provided that L494I is present in C5. However, the negative charge of Asp⁶⁰¹ did not appear to be favoured in the long term, as evidenced by the outgrowth of Asn⁶⁰¹-containing clones, and WT levels of gp120-gp41 association for the T138N/L494I/K601Nclone. Cell-cell fusion and viral replication were also restored by a short ΔN¹³⁹INN deletion in V1 operating in conjunction with Asn⁶⁰¹ within the DSR. Interestingly, WT levels of gp120-gp41 association were not achieved by the replication-competentΔN¹³⁹INN/K601N clone, suggesting that reversion in this case involves the restoration of Envfusogenicity in the context of an unstable gp120-gp41 complex. The fusion and gp120-gp41 association phenotype of ΔN¹³⁹INN/K601N was largely recapitulated by the S144N/K601N combination, suggesting that loss of the Asn¹⁴² glycan is sufficient to restore fusion function when Asn⁶⁰¹ is present in the DSR. Notably, mutations designed to ablate the remaining PNGSs within V1V2 at positions 141, 156, 160 and 186, did not restore function to Asn⁶⁰¹-containing Envs, indicating a specific functional linkage between the DSR and the Asn¹³⁶ and Asn¹⁴² glycosylation sites.

X-ray crystallography has indicated that V1V2 comprises a conserved 4-stranded β-sheet minidomain that is stabilized by 2 interstrand disulfide bonds (Cys¹²⁶-Cys¹⁹⁶ and Cys¹³¹-Cys¹⁵⁷). The highly variable segments and PNGSs of V1V2 are for the most part contained within the connecting loops (FIG. 16A) [7]. A model of glycosylated AD8 V1V2 based on the CAP45 V1V2 structure [7] indicates that 6 N-linked glycans would encompass this minidomain in a hydrophilic shell (FIG. 16A). The most obvious changes due to T138N and ΔN¹³⁹INN would be the loss of hydrophilic glycan bulk (˜1840 Å³ per high-mannose glycan; see [71]) and, for the latter, a shortening of the V1 loop (FIG. 16A). N-linked glycans have been found to induce local order in the backbone of protein loop regions by destabilizing the unfolded state, the increased entropy of the flexible glycan side chain compensating for the decreased entropy of the protein backbone [72,73,74]. The loss of the Asn¹³⁶ and Asn¹⁴² glycans may lead to localized disorder and structural change and/or instability within V1, which in turn affects other structural elements of the gp120-gp41 complex. Cryoelectron tomography indicates that the Asn¹³⁶ and Asn¹⁴²V1 glycans, shown here to be functionally linked to the DSR, would be located at the apex of the trimeric Env spike whereas the gp120-gp41 association synapse is underneath the gp120 trimer [8,12]. An allosteric mechanism whereby changes in V1 can affect the structure of the distal gp120-gp41 association site is suggested by the architecture of gp120, wherein 3 structurally plastic layers of the core domain are linked to the N- and C-terminal gp41-associating segments via an invariant 7-stranded β-sandwich [8] (FIG. 16B). Layer 1 connects the β2-β3 hairpin that forms the base of V1V2 via the β-sandwich to the gp120 N- and C-terminal segments (FIG. 16A, B). Thus changes in V1V2 structure and/or stability due to the loss of the Asn¹³⁶ or Asn¹⁴² glycans may cause a distortion or displacement of layer 1, which is translated to the N- and C-terminal gp41-interacting segments via the β-sandwich. A result of these changes in gp120 is the accommodation of the shorter branched side chains of Asp⁶⁰¹ and Asn⁶⁰¹ within the association site.

A mechanism for suppression of the K601D fusion phenotype was indicated by an analysis of combination mutants comprising T138N, L494I and ΔN¹³⁹INN with various DSR mutations known to affect glycoprotein association and/or fusion function [27,28]. The T138N/L494I combination rendered fusion function largely independent of the DSR residues Leu⁵⁹³ and Lys⁶⁰¹, whereas less dependence on Lys⁶⁰¹ was observed with ΔN¹³⁹INN. The boosts in fusion function provided to L593V and K601D by these V1 and C5 mutations generally correlated with the restoration of gp120-gp41 association. In the case of W596L, the T138N and ΔN¹³⁹INN glycan mutations were sufficient to confer WT fusion levels, but this did not involve changes to the WT-like gp120-gp41 association phenotype of W596L. The fusion gains conferred to W596L by T138N and ΔN¹³⁹INN may involve transduction of a receptor-induced activation signal from gp120 to gp41 through the association site in manner that is less dependent on Trp⁵⁹⁶. The earlier finding that W596L blocks sCD4-induced formation of the gp41 prehairpin intermediate indicates that Trp⁵⁹⁶ can play a role in receptor-triggered gp41 activation [29]. Overall, these data suggest that the glycan changes observed here involve a structural remodeling of the gp120-gp41 association interface such that Env function is less dependent on particular gp120-contact residues within the DSR, e.g. Trp⁵⁹⁶ [27,28]. The maintenance of a functional Env complex in these cases may involve alternative inter-subunit contacts mediated by other DSR residues implicated in gp120 association [e.g. Gln⁵⁹¹, Gly⁵⁹⁷, Thr⁶⁰⁶ and Trp⁶¹⁰ [27,28,75]], and/or other regions of gp41 that appear to interact with gp120, including the fusion-peptide proximal segment [76], HR1 [77,78], and HR2 [75]. Interestingly, it was observed that the C34 HR2 peptide analog inhibited fusion mediated by WT, T138N/L494I/K601N and ΔN¹³⁹INN/K601N Env to the same extent, indicating that the prefusion gp41-to-6-helix bundle refolding process was not affected by these amino acid changes in gp120 and gp41.

The V1V2 mini domain has been shown to modulate the accessibility and/or conformation of the CD4-binding site and V3 loop [38,79,80,81,82], the latter of which mediates chemokine receptor binding. However, our data argue against a reversion mechanism involving altered gp120-receptor interactions since the sCD4-binding abilities of gp120 molecules bearing 2^{nd} and 3^{rd} site mutations in various combinations were not significantly different to the WT. Furthermore, the T138N/L494I/K601N and ΔN¹³⁹INN/K601N mutants were able to mediate WT levels of fusion with targets expressing various CCR5 mutants, including the low affinity Y14N coreceptor, thereby also ruling out altered gp120-CCR5 interactions as a potential reversion mechanism. It should however be noted that ΔN¹³⁹INN/K601N-mediated fusion appeared to be subtly more resistant to sCD4 inhibition in comparison to WT and T138N/L494I/K601N. ΔN¹³⁹INN/K601N exhibits weaker gp120-gp41 association than WT and T138N/L494I/K601N despite near-WT replication and fusion competence and WT-like sCD4 binding characteristics for soluble gp120 containing ΔN¹³⁹INN. sCD4 has been shown to primarily inhibit HIV-1 infection by inducing a transiently activated glycoprotein complex that rapidly undergoes irreversible conformational changes linked to a loss of function [83]. This sCD4-mediated inactivation correlates with a rapid decay in exposure of the HR1 coiled coil groove of the fusion-activated gp41 prehairpin intermediate. It may be that the apparently looser association between gp120 and gp41 of ΔN¹³⁹INN/K601N alters the conformational pathway of the sCD4-induced state to enable maintenance of fusion competence. Overall, these data lend support to the idea that the changes in V1 specifically impact on the gp120-gp41 association site.

A striking feature of gp120-gp41 is the occlusion of the conserved protein surface by a glycan shield comprising ˜24 N-linked glycans on gp120 and 4-5 on gp41 [3,84,85,86,87]. An evolving glycan shield is an important mediator of viral escape from NAbs where an increase in the number and/or a change in the position of PNGSsalter NAb sensitivity [35]. A number of studies have indicated that V1V2 plays a particularly important role in regulating neutralization resistance [36,38,41,42], which generally correlates with V1V2 elongation and insertion of PNGSs, in some cases at and C-terminal to position 136 (FIG. 16C) [34,37,38,39,40,42,43,44]. The data herein indicates that subtle changes to the glycan shield in V1 impact on neutralization by glycan-dependent brNAbs. For example, the Asn¹³⁶ and Asn¹⁴¹/Asn¹⁴² glycan mutations were found to increase the sensitivity of HIV-1 pseudovirions to the 2G12 NAb, which is dependent on high-mannose glycans on the outer face of gp120, including those attached to Asn²⁹⁵ and Asn³³² at the base of V3, as well as Asn³³⁹, Asn³⁸⁶ and Asn³⁹² [58,59,60,61,62,63] (FIG. 16B). Neutralization epitopes within V3 appear particularly sensitive to changes in V1V2, which is likely due to the proximity between these variable structures in the context of trimeric gp120-gp41 [10,12,38,79,81]. The Asn¹³⁶ and Asn¹⁴¹/Asn¹⁴²V1 glycan deletions may modulate the structure and/or accessibility the 2G12 glycan epitope by altering V1V2-V3 interactions in the context of trimeric gp120. Alternatively, the enhanced neutralization of T138N and ΔN¹³⁹INN by 2G12 may be a result of changes in the global antigenic structure of gp120-gp41, as was suggested by the finding that the V1 glycan deletions slightly enhanced the neutralization potency of polyclonal HIVIG. That V1V2 can regulate global antigenic structure via changes in the glycan shield has been suggested by the finding that glycosylation changes within gp120 at 197, 234, 295 and 301 contribute to the restoration of infectivity to viruses from which the entire V1V2 region had been deleted [88]. We also observed that ΔN¹³⁹INN led to increased sensitivity to neutralization by NAb PG16, whose complex epitope includes the V1V2 glycans at Asn¹⁵⁶ and Asn¹⁶⁰ and is also influenced by residues in V3 [7,64,65]. The AD8 V1V2 model suggests that the Asn¹⁴² glycan is proximal to the Asn¹⁵⁶ glycan, implying that the N¹³⁹INN deletion may relieve a steric constraint that enables better epitope access for PG16. Alternatively, or additionally, changes in the disposition of V3, as was implied by the increased sensitivity of ΔN¹³⁹INN to neutralization by 2G12, may contribute to the increased neutralization efficacy of PG16. Overall, our data indicate that the Asn¹³⁶ and Asn^{141/142} glycans of V1 can modulate local (PG16) and remote (2G12) glycan-dependent neutralization epitopes as well as the global antigenic structure of Env (HIVIG) and that these changes are functionally linked to a remodelling of the gp120-gp41 association site. Our data also imply that glycan shield evolution may indirectly affect the inhibitory potential of novel fusion blockers, such as PF-68742, for which the DSR is a component of their targeting mechanism [33]. Conversely, DSR sequence evolution driven by potential DSR-directed entry inhibitors may be associated with compensatory V1 glycan changes as described here and the coevolution of neutralization sensitivity.

As shown herein, changes at the 136 and 142 V1 glycans that are associated with neutralization sensitivity appear to remodel the gp120-gp41 association site, rendering certain highly conserved gp120-contact residues (i.e. Leu⁵⁹³, Trp⁵⁹⁶ and Lys⁶⁰¹) less important for gp120 association and fusion function, and thereby implying that alternative gp120-gp41 contact residues become utilized for these functions. The allosteric modulation of the conserved DSR-C1-05 synapse by distal V1 glycans may represent a mechanism whereby functionally relevant gp120-gp41 association is maintained as the virus acquires neutralization resistance due to the evolution of its glycan shield.

### Example 3

### Materials and Methods

### Env Expression Vectors

The cytomegalovirus promoter-driven HIV-1_{AD8}Env expression vector, pΔKAD8env, is described above, as are the ΔN¹³⁹INN- and W596L/K601H/D674E mutated versions. The ΔN¹³⁹INN and W596L/K601H/D674E mutations were combined in a single pΔKAD8env vector by replacing the PpuMI DNA fragment of pΔKAD8env-ΔN¹³⁹INN with that of pΔKAD8env-WL/KH/DE to give pΔKAD8env-ΔN¹³⁹INN/WL/KH/DE, referred to as ΔNINN/WL/KH/DE (Figure. 18).

### Env-Pseudotyped Luciferase Reporter Viruses

Env-pseudotyped luciferase reporter viruses were produced by cotransfecting 293T cells with pΔKAD8env vectors plus the luciferase reporter virus vector, pNL4.3.Luc.R⁻E⁻ using Fugene 6. The infectivity of pseudotyped viruses was determined in U87.CD4.CCR5 cells.

### Neutralization Assay

A solution of Env-pseudotyped luciferase reporter viruses determined previously to give 1-2×10⁵ relative light units (RLU) following infection of U87.CD4.CCR5 cells was mixed with an equal volume of serially diluted IgG and incubated for 1 h at 37° C. One hundred microliters of the virus-IgG mixtures was then added to U87.CD4.CCR5 cells (10⁴ cells per well of a 96-well tissue culture plate, 100 microlitres) and incubated for 2 days prior to lysis and assay for luciferase activity (Promega, Madison, Wis.). Neutralizing activities for antibody samples were measured in triplicate and reported as the average percent luciferase activity. Purified IgG of monoclonal brNAbs 4E10, 2G12 and IgGb12 were obtained from Polymun Scientific (Austria). PGT121 and PGT126 were obtained from the International AIDS Vaccine Initiative.

### Results

### Infectivity of ΔNINN/WL/KH/DE

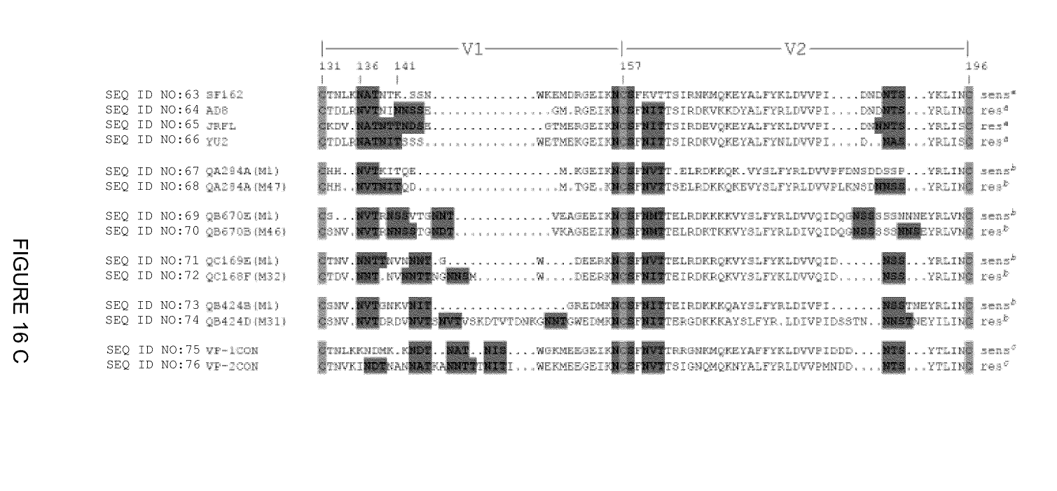

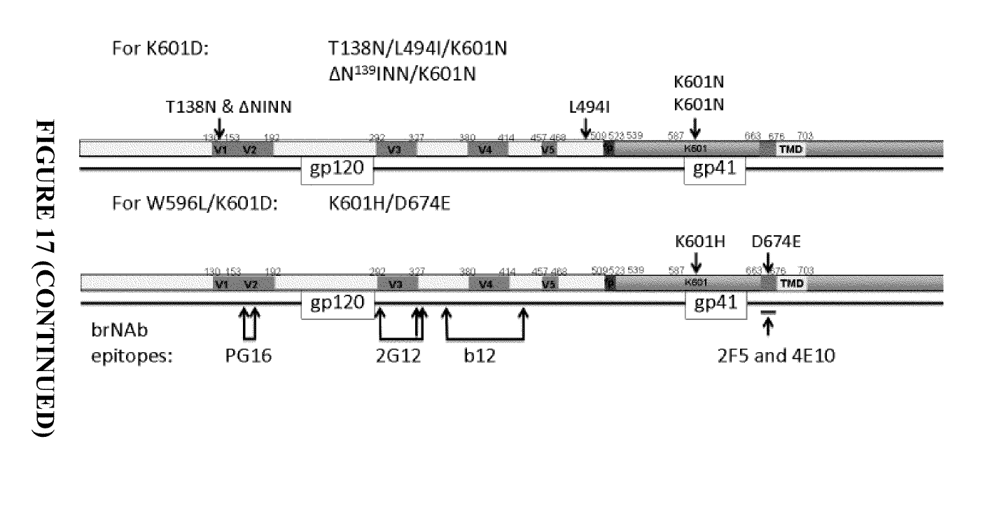

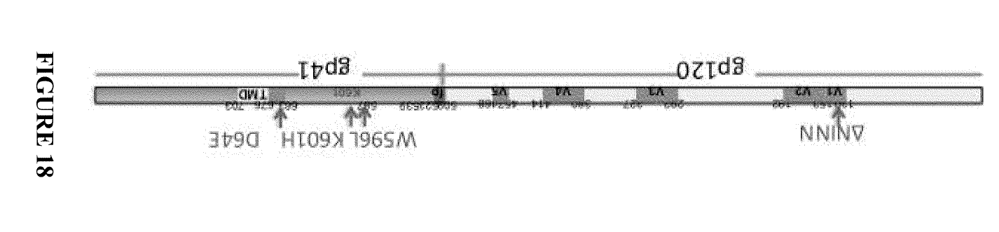

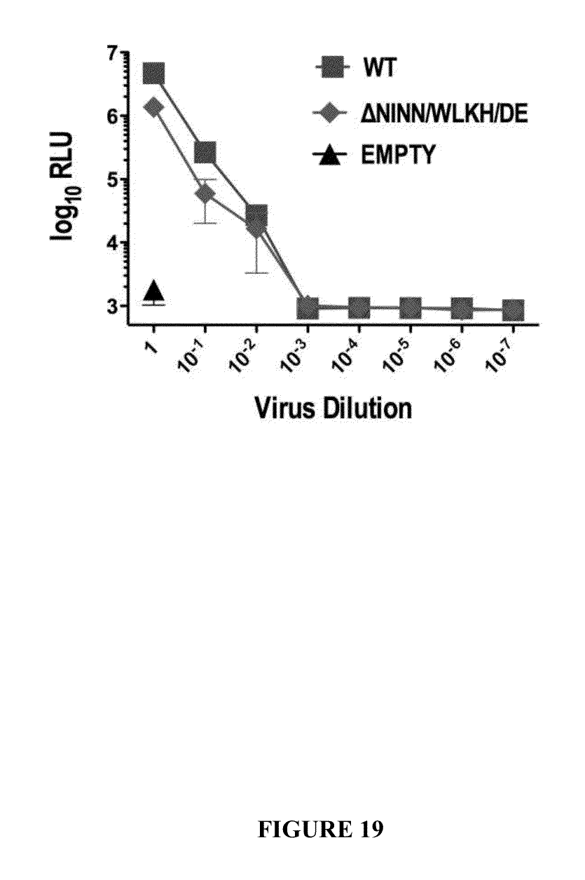

Transfection supernatants containing WT or ΔNINN/WL/KH/DE Env pseudotyped luciferase reporter viruses were serially diluted and then used to infect U87.CD4.CCR5 target cells. The cells were lysed at 48-h post-infection and luciferase activity measured. FIG. 19 shows that the infectivity of ΔNINN/WL/KH/DE Env pseudotyped viruses is ˜0.5 log₁₀ lower than that of WT. Luciferase reporter viruses lacking Env (empty) were included as a negative control.

Sensitivity of ΔNINN/WL/KH/DE Env Pseudotyped Luciferase Reporter Viruses to brNAbs

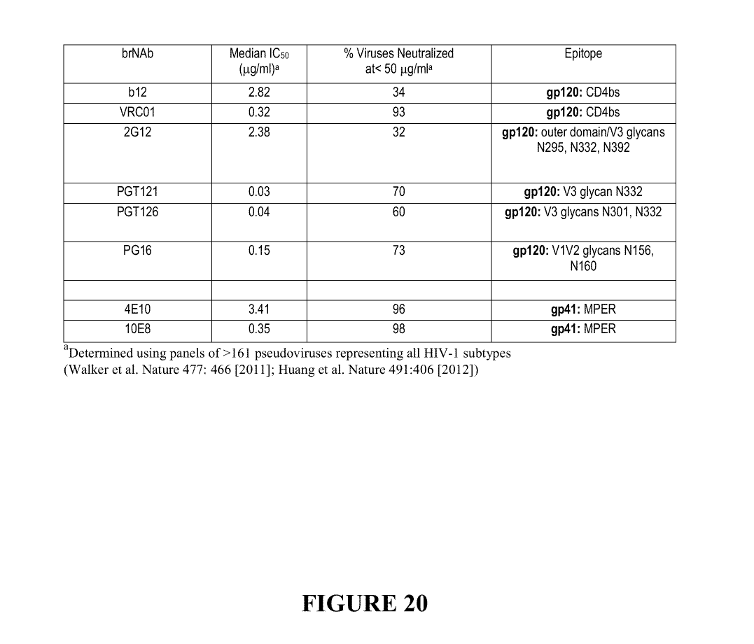

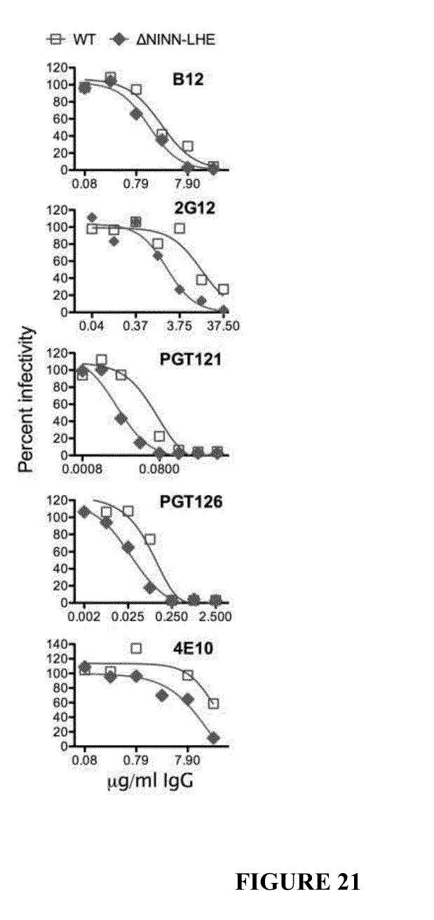

The ΔNINN/WL/KH/DE mutation conferred sensitivity to various brNAbs whose properties are listed in FIG. 20. Whereas WT and ΔNINN/WL/KH/DE reporter viruses were neutralized to similar extents by the CD4 binding site directed brNAb, IgGb12, ΔNINN/WL/KH/DE was neutralized more effectively by the oligomannose glycan-dependent gp120-directed brNAbs, 2G12, PGT121 and PGT126, and the MPER-directed brNAb 4E10 (FIG. 21). These data indicate that the combination mutation, ΔNINN/WL/KH/DE, sensitizes the viral gp120-gp41 complex to neutralization by oligomannose glycan-dependent gp120-directed brNAbs (2G12, PGT121 and PGT126), and an MPER-directed brNAb (4E10).

The combination of the ΔN¹³⁹INN gp120 V1 mutation and the WL/KH/DE gp41 mutations resulted in the sensitization of the viral gp120-gp41 complex to neutralization by oligomannose glycan-dependent gp120-directed brNAbs and an MPER-directed brNAb. It is proposed that the ΔN¹³⁹INN mutation alters the conformation of the glycan shield of gp120, enabling better access for brNAbs to oligomannose-dependent epitopes on the Env surface, while the gp41 mutations within the gp120-gp41 association site and MPER may lead to a more open Env structure, and exposure of brNAb epitopes within the MPER. Alternatively or additionally, WL/KH/DE may be associated with structural change in the MPER itself, with increased MPER flexibility and/or altered membrane interactions facilitating paratope-mediated extraction of the 4E10 epitopes from the envelope. These proposed changes to the MPER may be present in cell-free virions or could occur during the virus-cell fusion process.

### Example 4

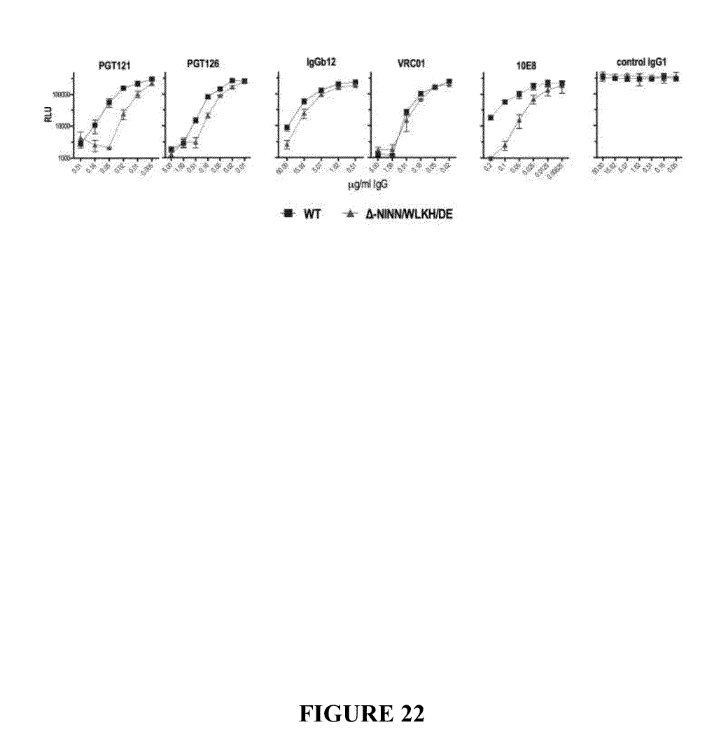

The neutralization assays were repeated using additional brNAbs: VRC01, directed to the CD4-binding site of gp120 and 10E8, directed to the MPER of gp41 (1,2). The VRCO1 and 10E8brNAbs are of particular interest because they exhibit greater HIV-1 neutralization potency and neutralization breadth when compared with IgGb12 and 4E10, respectively. The data presented in FIG. 22 show that the ΔNINN/WL/KH/DE Env-containing pseudovirus is markedly more sensitive to neutralization by the MPER directed 10E8 brNAb but not to the CD4 binding site directed brNAb, VRC01. Consistent with the data presented in FIG. 21, ΔNINN/WL/KH/DE viruses were more sensitive to the glycan dependent brNAbs, PGT121 and PGT126 than was WT (FIG. 22). Again, IgGb12 neutralized WT and mutant viruses to similar extents although ΔNINN/WL/KH/DE appeared marginally more sensitive to this brNAb. Finally, control IgG1 obtained from an individual who had not been exposed to HIV-1 did not neutralize WT nor ΔNINN/WL/KH/DE viruses confirming that neutralization is dependent on the Envgp120/gp41 specificity of the brNAbs for HIV-1.

These data indicate that the combination mutation, ΔNINN/WL/KH/DE, sensitizes the viral gp120-gp41 complex to neutralization by oligomannose glycan-dependent gp120-directed brNAbs(PGT121 and PGT126) and by an MPER-directed brNAb (10E8).

It was proposed that incorporation of ΔNINN/WL/KH/DE into a human immunodeficiency virus like particle (HIVLP) immunogen enhanced the presentation of oligomannose-dependent epitopes in gp120 as well as MPER epitopes in gp41 in a quasi-native context and in conformations that would promote the production of brNAbs in vaccinated mammals including humans. To test this proposal, a pcDNA3-based GagPol expression vector, pcGagPolVpu was constructed for HIVLP production. The vector contains the HIV-1_{NL4.3}(3) gagpol-vif-vpu genomic fragment. Translation termination codons were introduced to the vif and vpr open reading frames close to the initiation codons in order to block their expression. Furthermore, a gene fragment encoding the Rev-responsive element was PCR-amplified using the pNL4.3 infectious clone as template and ligated into the unique XbaI site in pcGagPolVpu downstream of the HIV-1 coding region. The inclusion of the Rev responsive element is to enable nuclear export of mRNAs encoding Gag, GagPol and Vpu, which is mediated by the viral protein Rev.

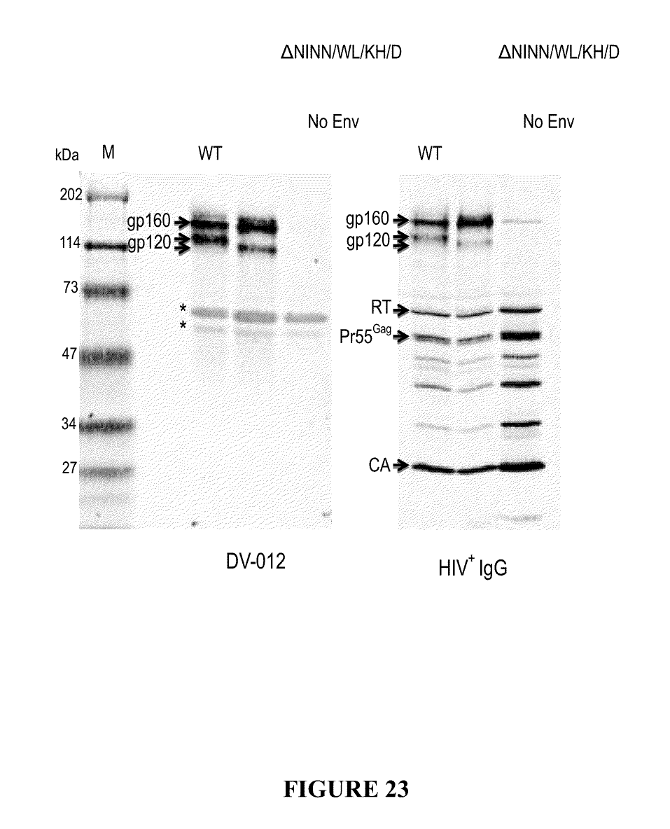

HIVLPs were produced by co-transfecting 293T cells with pcGagPolVpu (1 μg) plus pΔKAD8-WT (1 μg), or pcGagPolVpu (1 μg) plus pΔKAD8-ΔNINN/WL/KH/DE (1 μg) using the Fugene HD procedure. Control HIVLPs lacking Env were produced by cotransfecting 293T cells with pcGagPolVpu (1 μg) plus pCMV-Rev (1 μg), a vector that expresses the viral protein Rev from a cytomegalovirus promoter (4). At 4-8 h post-transfection, the culture medium was replaced with Optimem and the supernatants harvested at 72 h post-transfection. HIVLPs were partially purified by ultracentrifugation (25,000 rpm for 2 hr at 4° C. in a SW41 rotor) through a 1.5 ml sucrose cushion (25% sucrose in PBS). The pelleted virions were resuspended in PBS and then subjected to SDS-PAGE under reducing conditions followed by Western blotting with a sheep polyclonal antiserum raised to recombinant gp120 (DV-012) and IgG purified from the plasma of an HIV-1-infected individual (HIV+IgG). Western blotting with DV-012 shows that the HIVLPs contain mature gp120 as well as the uncleaved Env precursor, gp160. Blotting with HIV+IgG reveals the presence of Gag and GagPol cleavage products in addition to gp120 and gp160 (FIG. 23) indicative of the formation of intact HIVLP.

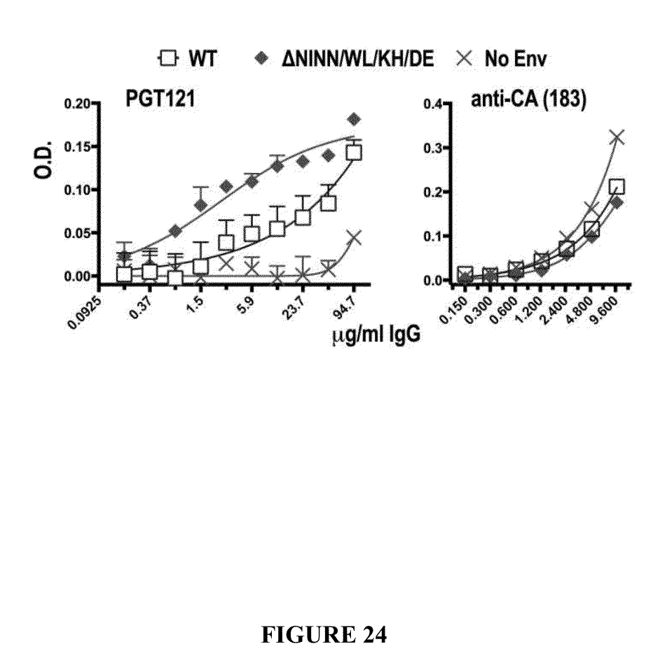

An enzyme linked immunoassay (ELISA) protocol was developed to determine whether the ΔNINN/WL/KH/DE mutations led to enhanced recognition of pseudovirion-incorporated Env by brNAbs. The volumes of HIVLP suspensions were adjusted with PBS to normalize the capsid (CA) based on the intensity of the CA band observed in the western blot. 96-well ELISA plates (NUNC) were coated with 50 μl HIVLP suspensions at 37° C. for 2 h. The plates were washed thrice with PBS and then blocked with 100 μl of a 3% bovine serum albumin-PBS solution at 37° C. for 1 h. The plates were again washed three times prior to the addition of a dilution series of PGT121 in 50 μl 5% skim milk powder-PBS. The plates were incubated for 1 h at 37° C. The plates were washed 6 times with KPL buffer lacking Tween 20 prior to the addition of 50 μhorseradish peroxidase conjugated rabbit immunoglobulins to human immunoglobulins (DAKO) in 5% skim milk powder-PBS. The plates were incubated at room temperature for 1 h and then washed 6 times with KPL buffer lacking Tween 20. The ELISA was developed with 3,3′,5,5′-tetramethylbenzidine in phosphate-citrate buffer (pH 5.0) and the reaction terminated with 1N HCl. The background absorbance at 620 nm was subtracted from the absorbance at 450 nm. The data (FIG. 24) show that PGT121 exhibits enhanced binding to ΔNINN/WL/KH/DE Env-containing HIVLPs relative to those containing WT Env. Background levels of PGT121 binding to HIVLPs lacking Env were observed, confirming the specificity of the ELISA.

The overall coating levels of WT, ΔNINN/WL/KH/DE and Env-deficient HIVLPs were compared in a modified ELISA employing the anti-CA mouse monoclonal antibody 183-H12-5C (183)(5). In this modified assay, plate-bound HIVLPs were treated with 1% Triton X100 at 4° C. for 1 h to permeabilize the cholesterol-rich envelope to allow mAb 183 to access the internal CA. The plate was washed three times with PBS and a mAb 183 dilution series (50 μl in 5% skim milk powder-PBS) added to the plates. The plates were incubated for 1 h at 37° C. The plates were washed 6 times with KPL buffer lacking Tween 20 prior to the addition of 50 μl horseradish peroxidase conjugated rabbit immunoglobulins to mouse immunoglobulins in 5% skim milk powder-PBS. The plates were incubated at room temperature for 1 h and then washed 6 times with KPL buffer lacking Tween 20. The ELISA was developed as described above. Comparable binding by mAb 183 to WT and ΔNINN/WL/KH/DE-containing HIVLPs as well as to HIVLPs lacking Env indicates comparable coating levels for the 3 HIVLP preparations. Therefore, the observed increased binding by PGT121 to ΔNINN/WL/KH/DE Env-containing HIVLPs is indeed consistent with the enhanced exposure of brNAb epitopes.

It is proposed that incorporation of ΔNINN/WL/KH/DE into a HIVLP immunogen enhances the presentation of oligomannose-dependent epitopes in gp120 and in conformations that promote the production of brNAbs to such epitopes in vaccinated mammals including humans. It is also proposed that incorporation of ΔNINN/WL/KH/DE in a quasi-native context of HIVLPs will improve the presentation of MPER-dependent epitopes in gp41 and in conformations that promote the production of brNAbs in vaccinated mammals including humans.

The disclosure of every patent, patent application, and publication cited herein is hereby incorporated herein by reference in its entirety.

Many modifications will be apparent to those skilled in the art without departing from the scope of the present invention.

TABLE 1
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

TABLE 2
Exemplary and Preferred Amino Acid Substitutions

| Original | | Preferred |
|---|---|---|
| Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

### BIBLIOGRAPHY 1

1. Liu J, Bartesaghi A, Borgnia M J, Sapiro G, Subramaniam S: Molecular architecture of native HIV-1 gp120 trimers. Nature 2008, 455:109-113.
2. White T A, Bartesaghi A, Borgnia M J, Meyerson J R, de la Cruz M J, Bess J W, Nandwani R, Hoxie J A, Lifson J D, Milne J L, Subramaniam S: Molecular architectures of trimeric SIV and HIV-1 envelope glycoproteins on intact viruses: strain-dependent variation in quaternary structure. PLoS Pathog 2010, 6:e1001249.
3. Checkley M A, Luttge B G, Freed E O: HIV-1 envelope glycoprotein biosynthesis, trafficking, and incorporation. J Mol Biol 2011, 410:582-608.
4. Melikyan G B: Membrane fusion mediated by human immunodeficiency virus envelope glycoprotein. Curr Top Membr 2011, 68:81-106.
5. Furuta R A, Wild C T, Weng Y, Weiss C D: Capture of an early fusion-active conformation of HIV-1 gp41. Nature Structural Biology 1998, 5:276-279.
6. He Y, Vassell R, Zaitseva M, Nguyen N, Yang Z, Weng Y, Weiss C D: Peptides trap the human immunodeficiency virus type 1 envelope glycoprotein fusion intermediate at two sites. J Virol 2003, 77:1666-1671.
7. Tran E E, Borgnia M J, Kuybeda O, Schauder D M, Bartesaghi A, Frank G A, Sapiro G, Milne J L, Subramaniam S: Structural mechanism of trimeric HIV-1 envelope glycoprotein activation. PLoS Pathog 2012, 8:e1002797.
8. Chan D C, Fass D, Berger J M, Kim P S: Core structure of gp41 from the HIV envelope glycoprotein. Cell 1997, 89:263-273.
9. Tan K, Liu J, Wang J, Shen S, Lu M: Atomic structure of a thermostable subdomain of HIV-1 gp41. Proc Natl Acad Sci USA 1997, 94:12303-12308.
10. Weissenhorn W, Dessen A, Harrison S C, Skehel J J, Wiley D C: Atomic structure of the ectodomain from HIV-1 gp41. Nature 1997, 387:426-430.
11. Binley J M, Sanders R W, Clas B, Schuelke N, Master A, Guo Y, Kajumo F, Anselma D J, Maddon P J, Olson W C, Moore J P: A recombinant human immunodeficiency virus type 1 envelope glycoprotein complex stabilized by an intermolecular disulfide bond between the gp120 and gp41 subunits is an antigenic mimic of the trimeric virion-associated structure. J Virol 2000, 74:627-643.
12. Helseth E, Olshevsky U, Furman C, Sodroski J: Human immunodeficiency virus type 1 gp120 envelope glycoprotein regions important for association with the gp41 transmembrane glycoprotein. J Virol 1991, 65:2119-2123.
13. Maerz A L, Drummer H E, Wilson K A, Poumbourios P: Functional Analysis of the Disulfide-Bonded Loop/Chain Reversal Region of Human Immunodeficiency Virus Type 1 gp41 Reveals a Critical Role in gp120-gp41 Association. J Virol 2001, 75:6635-6644.
14. Abrahamyan L G, Markosyan R M, Moore J P, Cohen F S, Melikyan G B: Human immunodeficiency virus type 1 env with an intersubunit disulfide bond engages coreceptors but requires bond reduction after engagement to induce fusion. J Virol 2003, 77:5829-5836.
15. Binley J M, Cayanan C S, Wiley C, Schulke N, Olson W C, Burton D R: Redox-triggered infection by disulfide-shackled human immunodeficiency virus type 1 pseudovirions. J Virol 2003, 77:5678-5684.
16. Bellamy-McIntyre A K, Bar S, Ludlow L, Drummer H E, Poumbourios P: Role for the disulfide-bonded region of human immunodeficiency virus type 1 gp41 in receptor-triggered activation of membrane fusion function. Biochem Biophys Res Commun 2010, 394:904-908.
17. Pancera M, Majeed S, Ban Y E, Chen L, Huang C C, Kong L, Kwon Y D, Stuckey J, Zhou T, Robinson J E, et al: Structure of HIV-1 gp120 with gp41-interactive region reveals layered envelope architecture and basis of conformational mobility. Proc Natl Acad Sci USA 2010, 107:1166-1171.
18. Yang X, Mahony E, Holm G H, Kassa A, Sodroski J: Role of the gp120 inner domain beta-sandwich in the interaction between the human immunodeficiency virus envelope glycoprotein subunits. Virology 2003, 313:117-125.
19. Finzi A, Xiang S H, Pacheco B, Wang L, Haight J, Kassa A, Danek B, Pancera M, Kwong P D, Sodroski J: Topological layers in the HIV-1 gp120 inner domain regulate gp41 interaction and CD4-triggered conformational transitions. Mol Cell 2010, 37:656-667.
20. Gach J S, Leaman D P, Zwick M B: Targeting HIV-1 gp41 in close proximity to the membrane using antibody and other molecules. Curr Top Med Chem 2011, 11:2997-3021.
21. Montero M, van Houten N E, Wang X, Scott J K: The membrane-proximal external region of the human immunodeficiency virus type 1 envelope: dominant site of antibody neutralization and target for vaccine design. Microbiol Mol Biol Rev 2008, 72:54-84.
22. Salzwedel K, West J T, Hunter E: A conserved tryptophan-rich motif in the membrane-proximal region of the human immunodeficiency virus type 1 gp41 ectodomain is important for Env-mediated fusion and virus infectivity. J Virol 1999, 73:2469-2480.
23. Suarez T, Gallaher W R, Agirre A, Goni F M, Nieva J L: Membrane interface-interacting sequences within the ectodomain of the human immunodeficiency virus type 1 envelope glycoprotein: putative role during viral fusion. J Virol 2000, 74:8038-8047.
24. Bellamy-McIntyre A K, Lay C S, Baar S, Maerz A L, Talbo G H, Drummer H E,
Poumbourios P: Functional links between the fusion peptide-proximal polar segment and membrane-proximal region of human immunodeficiency virus gp41 in distinct phases of membrane fusion. J Biol Chem 2007, 282:23104-23116.
25. Sun Z Y, Oh K J, Kim M, Yu J, Brusic V, Song L, Qiao Z, Wang J H, Wagner G, Reinherz E L: HIV-1 Broadly Neutralizing Antibody Extracts Its Epitope from a Kinked gp41 Ectodomain Region on the Viral Membrane. Immunity 2008, 28:52-63.
26. Buzon V, Natraj an G, Schibli D, Campelo F, Kozlov M M, Weissenhorn W: Crystal structure of HIV-1 gp41 including both fusion peptide and membrane proximal external regions. PLoS Pathog 2010, 6:e1000880.
27. Lay C S, Wilson K A, Kobe B, Kemp B E, Drummer H E, Poumbourios P: Expression and biochemical analysis of the entire HIV-2 gp41 ectodomain: determinants of stability map to N- and C-terminal sequences outside the 6-helix bundle core. FEBS Lett 2004, 567:183-188.
28. Lay C S, Ludlow L E, Stapleton D, Bellamy-McIntyre A K, Ramsland P A, Drummer H E, Poumbourios P: Role for the terminal clasp of HIV-1 gp41 glycoprotein in the initiation of membrane fusion. J Biol Chem 2011, 286:41331-41343.
29. Noah E, Biron Z, Naider F, Arshava B, Anglister J: The membrane proximal external region of the HIV-1 envelope glycoprotein gp41 contributes to the stabilization of the six-helix bundle formed with a matching N′ peptide. Biochemistry 2008, 47:6782-6792.
30. Ferrantelli F, Hofmann-Lehmann R, Rasmussen R A, Wang T, Xu W, Li P L, Montefiori D C, Cavacini L A, Katinger H, Stiegler G, et al: Post-exposure prophylaxis with human monoclonal antibodies prevented SHIV89.6P infection or disease in neonatal macaques. AIDS 2003, 17:301-309.
31. Ofek G, Guenaga F J, Schief W R, Skinner J, Baker D, Wyatt R, Kwong P D: Elicitation of structure-specific antibodies by epitope scaffolds. Proc Natl Acad Sci USA 2010, 107:17880-17887.
32. Phogat S, Svehla K, Tang M, Spadaccini A, Muller J, Mascola J, Berkower I, Wyatt R: Analysis of the human immunodeficiency virus type 1 gp41 membrane proximal external region arrayed on hepatitis B surface antigen particles. Virology 2008, 373:72-84.
33. Poumbourios P, Maerz A L, Drummer H E: Functional evolution of the HIV-1 envelope glycoprotein 120 association site of glycoprotein 41. J Biol Chem 2003, 278:42149-42160.
34. Hubner W, McNerney G P, Chen P, Dale B M, Gordon R E, Chuang F Y, Li X D, Asmuth D M, Huser T, Chen B K: Quantitative 3D video microscopy of HIV transfer across T cell virological synapses. Science 2009, 323:1743-1747.
35. Martin N, Welsch S, Jolly C, Briggs J A, Vaux D, Sattentau Q J: Virological synapse-mediated spread of human immunodeficiency virus type 1 between T cells is sensitive to entry inhibition. J Virol 2010, 84:3516-3527.
36. Davis M R, Jiang J, Zhou J, Freed E O, Aiken C: A mutation in the human immunodeficiency virus type 1 Gag protein destabilizes the interaction of the envelope protein subunits gp120 and gp41. J Virol 2006, 80:2405-2417.
37. Jiang J, Aiken C: Maturation-dependent human immunodeficiency virus type 1 particle fusion requires a carboxyl-terminal region of the gp41 cytoplasmic tail. J Virol 2007, 81:9999-10008.
38. Joyner A S, Willis J R, Crowe J E, Jr., Aiken C: Maturation-induced cloaking of neutralization epitopes on HIV-1 particles. PLoS Pathog 2011, 7:e1002234.
39. Murakami T, Ablan S, Freed E O, Tanaka Y: Regulation of human immunodeficiency virus type 1 Env-mediated membrane fusion by viral protease activity. J Virol 2004, 78:1026-1031.
40. Chan D C, Chutkowski C T, Kim P S: Evidence that a prominent cavity in the coiled coil of HIV type 1 gp41 is an attractive drug target. Proc Natl Acad Sci USA 1998, 95:15613-15617.
41. Haim H, Si Z, Madani N, Wang L, Courter J R, Princiotto A, Kassa A, DeGrace M, McGee-Estrada K, Mefford M, et al: Soluble CD4 and CD4-mimetic compounds inhibit HIV-1 infection by induction of a short-lived activated state. PLoS Pathog 2009, 5:e1000360.
42. Dale B M, McNerney G P, Thompson D L, Hubner W, de Los Reyes K, Chuang F Y, Huser T, Chen B K: Cell-to-cell transfer of HIV-1 via virological synapses leads to endosomal virion maturation that activates viral membrane fusion. Cell Host Microbe 2011, 10:551-562.
43. Jolly C, Kashefi K, Hollinshead M, Sattentau Q J: HIV-1 cell to cell transfer across an Env-induced, actin-dependent synapse. J Exp Med 2004, 199:283-293.
44. Finnegan C M, Berg W, Lewis G K, DeVico A L: Antigenic properties of the human immunodeficiency virus transmembrane glycoprotein during cell-cell fusion. J Virol 2002, 76:12123-12134.
45. Sattentau Q J, Zolla-Pazner S, Poignard P: Epitope exposure on functional, oligomeric HIV-1 gp41 molecules. Virology 1995, 206:713-717.
46. Chakrabarti B K, Walker L M, Guenaga J F, Ghobbeh A, Poignard P, Burton D R, Wyatt R T: Direct Antibody Access to the HIV-1 MPER Positively Correlates with Neutralization Sensitivity. J Virol 2011.
47. Blish C A, Nguyen M A, Overbaugh J: Enhancing exposure of HIV-1 neutralization epitopes through mutations in gp41. PLoS Med 2008, 5:e9.
48. Leavitt M, Park E J, Sidorov I A, Dimitrov D S, Quinnan G V, Jr.: Concordant modulation of neutralization resistance and high infectivity of the primary human immunodeficiency virus type 1 MN strain and definition of a potential gp41 binding site in gp120. J Virol 2003, 77:560-570.
49. Wang W, De Feo C J, Zhuang M, Vassell R, Weiss C D: Selection with a peptide fusion inhibitor corresponding to the first heptad repeat of HIV-1 gp41 identifies two genetic pathways conferring cross-resistance to peptide fusion inhibitors corresponding to the first and second heptad repeats (HR1 and HR2) of gp41. J Virol 2011, 85:12929-12938.
50. Chaillon A, Braibant M, Moreau T, Thenin S, Moreau A, Autran B, Barin F: The V1V2 domain and an N-linked glycosylation site in the V3 loop of the HIV-1 envelope glycoprotein modulate neutralization sensitivity to the human broadly neutralizing antibody 2G12. J Virol 2011, 85:3642-3648.
51. Doores K J, Burton D R: Variable loop glycan dependency of the broad and potent HIV-1-neutralizing antibodies PG9 and PG16. J Virol 2010, 84:10510-10521.
52. Krachmarov C P, Honnen W J, Kayman S C, Gorny M K, Zolla-Pazner S, Pinter A: Factors determining the breadth and potency of neutralization by V3-specific human monoclonal antibodies derived from subjects infected with clade A or clade B strains of human immunodeficiency virus type 1. J Virol 2006, 80:7127-7135.
53. Pinter A, Honnen W J, He Y, Gorny M K, Zolla-Pazner S, Kayman S C: The V1/V2 domain of gp120 is a global regulator of the sensitivity of primary human immunodeficiency virus type 1 isolates to neutralization by antibodies commonly induced upon infection. J Virol 2004, 78:5205-5215.
54. Polzer S, Dittmar M T, Schmitz H, Schreiber M: The N-linked glycan g15 within the V3 loop of the HIV-1 external glycoprotein gp120 affects coreceptor usage, cellular tropism, and neutralization. Virology 2002, 304:70-80.
55. Sagar M, Wu X, Lee S, Overbaugh J: Human immunodeficiency virus type 1 V1-V2 envelope loop sequences expand and add glycosylation sites over the course of infection, and these modifications affect antibody neutralization sensitivity. J Virol 2006, 80:9586-9598.
56. Zhang P F, Bouma P, Park E J, Margolick J B, Robinson J E, Zolla-Pazner S, Flora M N,
Quinnan G V, Jr.: A variable region 3 (V3) mutation determines a global neutralization phenotype and CD4-independent infectivity of a human immunodeficiency virus type 1 envelope associated with a broadly cross-reactive, primary virus-neutralizing antibody response. J Virol 2002, 76:644-655.
57. Hoffman N G, Seillier-Moiseiwitsch F, Ahn J, Walker J M, Swanstrom R: Variability in the human immunodeficiency virus type 1 gp120 Env protein linked to phenotype-associated changes in the V3 loop. J Virol 2002, 76:3852-3864.
58. Hwang S S, Boyle T J, Lyerly H K, Cullen B R: Identification of the envelope V3 loop as the primary determinant of cell tropism in HIV-1. Science 1991, 253:71-74.
59. Nabatov A A, Pollakis G, Linnemann T, Kliphius A, Chalaby M I, Paxton W A: Intrapatient alterations in the human immunodeficiency virus type 1 gp120 V1V2 and V3 regions differentially modulate coreceptor usage, virus inhibition by C C/CXC chemokines, soluble CD4, and the b12 and 2G12 monoclonal antibodies. J Virol 2004, 78:524-530.
60. Pastore C, Nedellec R, Ramos A, Pontow S, Ratner L, Mosier D E: Human immunodeficiency virus type 1 coreceptor switching: V1/V2 gain-of-fitness mutations compensate for V3 loss-of-fitness mutations. J Virol 2006, 80:750-758.
61. Binley J M, Wrin T, Korber B, Zwick M B, Wang M, Chappey C, Stiegler G, Kunert R, Zolla-Pazner S, Katinger H, et al: Comprehensive cross-clade neutralization analysis of a panel of anti-human immunodeficiency virus type 1 monoclonal antibodies. J Virol 2004, 78:13232-13252.
62. Guenaga J, Wyatt R T: Structure-guided alterations of the gp41-directed HIV-1 broadly neutralizing antibody 2F5 reveal new properties regarding its neutralizing function. PLoS Pathog 2012, 8:e1002806.
63. Muster T, Steindl F, Purtscher M, Trkola A, Klima A, Himmler G, Ruker F, Katinger H: A conserved neutralizing epitope on gp41 of human immunodeficiency virus type 1. J Virol 1993, 67:6642-6647.
64. Zwick M B, Labrijn A F, Wang M, Spenlehauer C, Saphire E O, Binley J M, Moore J P, Stiegler G, Katinger H, Burton D R, Parren P W: Broadly neutralizing antibodies targeted to the membrane-proximal external region of human immunodeficiency virus type 1 glycoprotein gp41. J Virol 2001, 75:10892-10905.
65. Dennison S M, Stewart S M, Stempel K C, Liao H X, Haynes B F, Alam S M: Stable docking of neutralizing human immunodeficiency virus type 1 gp41 membrane-proximal external region monoclonal antibodies 2F5 and 4E10 is dependent on the membrane immersion depth of their epitope regions. J Virol 2009, 83:10211-10223.
66. Kamdem Toukam D, Tenbusch M, Stang A, Temchura V, Storcksdieck Genannt Bonsmann M, Grewe B, Koch S, Meyerhans A, Nchinda G, Kaptue L, Uberla K: Targeting antibody responses to the membrane proximal external region of the envelope glycoprotein of human immunodeficiency virus. PLoS One 2012, 7:e38068.
67. Kim M, Qiao Z, Yu J, Montefiori D, Reinherz E L: Immunogenicity of recombinant human immunodeficiency virus type 1-like particles expressing gp41 derivatives in a pre-fusion state. Vaccine 2007, 25:5102-5114.
68. Matyas G R, Wieczorek L, Beck Z, Ochsenbauer-Jambor C, Kappes J C, Michael N L, Polonis V R, Alving C R: Neutralizing antibodies induced by liposomal HIV-1 glycoprotein 41 peptide simultaneously bind to both the 2F5 or 4E10 epitope and lipid epitopes. AIDS 2009, 23:2069-2077.
69. Watson D S, Platt V M, Cao L, Venditto V J, Szoka F C, Jr.: Antibody response to polyhistidine-tagged peptide and protein antigens attached to liposomes via lipid-linked nitrilotriacetic acid in mice. Clin Vaccine Immunol 2011, 18:289-297.
70. Theodore T S, Englund G, Buckler-White A, Buckler C E, Martin M A, Peden K W: Construction and characterization of a stable full-length macrophage-tropic HIV type 1 molecular clone that directs the production of high titers of progeny virions. AIDS Res Hum Retroviruses 1996, 12:191-194.
71. Bjorndal A, Deng H, Jansson M, Fiore J R, Colognesi C, Karlsson A, Albert J, Scarlatti G, Littman D R, Fenyo E M: Coreceptor usage of primary human immunodeficiency virus type 1 isolates varies according to biological phenotype. J Virol 1997, 71:7478-7487.
72. Chang L J, Urlacher V, Iwakuma T, Cui Y, Zucali J: Efficacy and safety analyses of a recombinant human immunodeficiency virus type 1 derived vector system. Gene Ther 1999, 6:715-728.
73. Gao F, Morrison S G, Robertson D L, Thornton C L, Craig S, Karlsson G, Sodroski J, Morgado M, Galvao-Castro B, von Briesen H, et al.: Molecular cloning and analysis of functional envelope genes from human immunodeficiency virus type 1 sequence subtypes A through G. The WHO and NIAID Networks for HIV Isolation and Characterization. J Virol 1996, 70:1651-1667.
74. Connor R I, Chen B K, Choe S, Landau N R: Vpr is required for efficient replication of human immunodeficiency virus type-1 in mononuclear phagocytes. Virology 1995, 206:935-944.
75. Abacioglu Y H, Fouts T R, Laman J D, Claassen E, Pincus S H, Moore J P, Roby C A, Kamin-Lewis R, Lewis G K: Epitope mapping and topology of baculovirus-expressed HIV-1 gp160 determined with a panel of murine monoclonal antibodies. AIDS Res Hum Retroviruses 1994, 10:371-381.
76. Page K A, Stearns S M, Littman D R: Analysis of mutations in the V3 domain of gp160 that affect fusion and infectivity. J Virol 1992, 66:524-533.
77. Takikawa S, Ishii K, Aizaki H, Suzuki T, Asakura H, Matsuura Y, Miyamura T: Cell fusion activity of hepatitis C virus envelope proteins. J Virol 2000, 74:5066-5074.
78. Deng H, Liu R, Ellmeier W, Choe S, Unutmaz D, Burkhart M, Di Marzio P, Marmon S, Sutton R E, Hill C M, et al: Identification of a major co-receptor for primary isolates of HIV-1. Nature 1996, 381:661-666.
79. Roben P, Moore J P, Thali M, Sodroski J, Barbas C F, 3rd, Burton D R: Recognition properties of a panel of human recombinant Fab fragments to the CD4 binding site of gp120 that show differing abilities to neutralize human immunodeficiency virus type 1. J Virol 1994, 68:4821-4828.
80. Zhou T, Xu L, Dey B, Hessell A J, Van Ryk D, Xiang S H, Yang X, Zhang M Y, Zwick M B, Arthos J, et al: Structural definition of a conserved neutralization epitope on HIV-1 gp120. Nature 2007, 445:732-737.
81. Derdeyn C A, Decker J M, Sfakianos J N, Wu X, O'Brien W A, Ratner L, Kappes J C, Shaw G M, Hunter E: Sensitivity of human immunodeficiency virus type 1 to the fusion inhibitor T-20 is modulated by coreceptor specificity defined by the V3 loop of gp120. J Virol 2000, 74:8358-8367.
82. Platt E J, Bilska M, Kozak S L, Kabat D, Montefiori D C: Evidence that ecotropic murine leukemia virus contamination in TZM-bl cells does not affect the outcome of neutralizing antibody assays with human immunodeficiency virus type 1. J Virol 2009, 83:8289-8292.
83. Wei X, Decker J M, Liu H, Zhang Z, Arani R B, Kilby J M, Saag M S, Wu X, Shaw G M, Kappes J C: Emergence of resistant human immunodeficiency virus type 1 in patients receiving fusion inhibitor (T-20) monotherapy. Antimicrob Agents Chemother 2002, 46:1896-1905.
84. Dorr P, Westby M, Dobbs S, Griffin P, Irvine B, Macartney M, Mori J, Rickett G, Smith-Burchnell C, Napier C, et al: Maraviroc (UK-427,857), a potent, orally bioavailable, and selective small-molecule inhibitor of chemokine receptor CCR5 with broad-spectrum anti-human immunodeficiency virus type 1 activity. Antimicrob Agents Chemother 2005, 49:4721-4732.
85. Huang C C, Lam S N, Acharya P, Tang M, Xiang S H, Hussan S S, Stanfield R L, Robinson J, Sodroski J, Wilson I A, et al: Structures of the CCR5 N terminus and of a tyrosine-sulfated antibody with HIV-1 gp120 and CD4. Science 2007, 317:1930-1934.
86. Du A P, Limal D, Semetey V, Dali H, Jolivet M, Desgranges C, Cung M T, Briand J P, Petit M C, Muller S: Structural and immunological characterisation of heteroclitic peptide analogues corresponding to the 600-612 region of the HIV envelope gp41 glycoprotein. J Mol Biol 2002, 323:503-521.

### BIBLIOGRAPHY 2

1. Checkley M A, Luttge B G, Freed E O (2011) HIV-1 envelope glycoprotein biosynthesis, trafficking, and incorporation. J Mol Biol 410: 582-608.
2. Melikyan G B (2011) Membrane fusion mediated by human immunodeficiency virus envelope glycoprotein. Curr Top Membr 68: 81-106.
3. Chen B, Vogan E M, Gong H, Skehel J J, Wiley D C, et al. (2005) Structure of an unliganded simian immunodeficiency virus gp120 core. Nature 433: 834-841.
4. Huang C C, Stricher F, Martin L, Decker J M, Majeed S, et al. (2005) Scorpion-toxin mimics of CD4 in complex with human immunodeficiency virus gp120 crystal structures, molecular mimicry, and neutralization breadth. Structure 13: 755-768.
5. Huang C C, Lam S N, Acharya P, Tang M, Xiang S H, et al. (2007) Structures of the CCR5 N terminus and of a tyrosine-sulfated antibody with HIV-1 gp120 and CD4. Science 317: 1930-1934.
6. Kwong P D, Wyatt R, Robinson J, Sweet R W, Sodroski J, et al. (1998) Structure of an HIV gp120 envelope glycoprotein in complex with the CD4 receptor and a neutralizing human antibody. Nature 393: 648-659.
7. McLellan J S, Pancera M, Carrico C, Gorman J, Julien J P, et al. (2011) Structure of HIV-1 gp120 V1/V2 domain with broadly neutralizing antibody PG9. Nature 480: 336-343.
8. Pancera M, Majeed S, Ban Y E, Chen L, Huang C C, et al. (2010) Structure of HIV-1 gp120 with gp41-interactive region reveals layered envelope architecture and basis of conformational mobility. Proc Natl Acad Sci USA 107: 1166-1171.
9. Basmaciogullari S, Babcock G J, Van Ryk D, Wojtowicz W, Sodroski J (2002) Identification of conserved and variable structures in the human immunodeficiency virus gp120 glycoprotein of importance for CXCR4 binding. J Virol 76: 10791-10800.
10. Liu J, Bartesaghi A, Borgnia M J, Sapiro G, Subramaniam S (2008) Molecular architecture of native HIV-1 gp120 trimers. Nature 455: 109-113.
11. Rizzuto C D, Wyatt R, Hernandez-Ramos N, Sun Y, Kwong P D, et al. (1998) A conserved HIV gp120 glycoprotein structure involved in chemokine receptor binding. Science 280: 1949-1953.
12. White T A, Bartesaghi A, Borgnia M J, Meyerson J R, de la Cruz M J, et al. (2010) Molecular architectures of trimeric SIV and HIV-1 envelope glycoproteins on intact viruses: strain-dependent variation in quaternary structure. PLoS Pathog 6: e1001249.
13. Colman P M, Lawrence M C (2003) The structural biology of type I viral membrane fusion. Nat Rev Mol Cell Biol 4: 309-319.
14. Eckert D M, Kim P S (2001) Mechanisms of viral membrane fusion and its inhibition. Annu Rev Biochem 70: 777-810.
15. Chang D K, Cheng S F, Trivedi V D (1999) Biophysical characterization of the structure of the amino-terminal region of gp41 of HIV-1. Implications on viral fusion mechanism. J Biol Chem 274: 5299-5309.
16. Furuta R A, Wild C T, Weng Y, Weiss C D (1998) Capture of an early fusion-active conformation of HIV-1 gp41. Nature Structural Biology 5: 276-279.
17. He Y, Vassell R, Zaitseva M, Nguyen N, Yang Z, et al. (2003) Peptides trap the human immunodeficiency virus type 1 envelope glycoprotein fusion intermediate at two sites. J Virol 77: 1666-1671.
18. Sackett K, Shai Y (2005) The HIV fusion peptide adopts intermolecular parallel beta-sheet structure in membranes when stabilized by the adjacent N-terminal heptad repeat: a 13C FTIR study. J Mol Biol 350: 790-805.
19. Lai A L, Moorthy A E, Li Y, Tamm L K (2012) Fusion activity of HIV gp41 fusion domain is related to its secondary structure and depth of membrane insertion in a cholesterol-dependent fashion. J Mol Biol 418: 3-15.
20. Caffrey M, Cai M, Kaufman J, Stahl S J, Wingfield P T, et al. (1998) Three-dimensional solution structure of the 44 kDa ectodomain of SIV gp41. EMBO J 17: 4572-4584.
21. Chan D C, Fass D, Berger J M, Kim P S (1997) Core structure of gp41 from the HIV envelope glycoprotein. Cell 89: 263-273.
22. Tan K, Liu J, Wang J, Shen S, Lu M (1997) Atomic structure of a thermostable subdomain of HIV-1 gp41. Proc Natl Acad Sci USA 94: 12303-12308.
23. Weissenhorn W, Dessen A, Harrison S C, Skehel J J, Wiley D C (1997) Atomic structure of the ectodomain from HIV-1 gp41. Nature 387: 426-430.
24. Yang Z N, Mueser T C, Kaufman J, Stahl S J, Wingfield P T, et al. (1999) The crystal structure of the SIV gp41 ectodomain at 1.47 A resolution. J Struct Biol 126: 131-144.
25. Binley J M, Sanders R W, Clas B, Schuelke N, Master A, et al. (2000) A recombinant human immunodeficiency virus type 1 envelope glycoprotein complex stabilized by an intermolecular disulfide bond between the gp120 and gp41 subunits is an antigenic mimic of the trimeric virion-associated structure. J Virol 74: 627-643.
26. Helseth E, Olshevsky U, Furman C, Sodroski J (1991) Human immunodeficiency virus type 1 gp120 envelope glycoprotein regions important for association with the gp41 transmembrane glycoprotein. J Virol 65: 2119-2123.
27. Maerz A L, Drummer H E, Wilson K A, Poumbourios P (2001) Functional Analysis of the Disulfide-Bonded Loop/Chain Reversal Region of Human Immunodeficiency Virus Type 1 gp41 Reveals a Critical Role in gp120-gp41 Association. J Virol 75: 6635-6644.
28. Poumbourios P, Maerz A L, Drummer H E (2003) Functional evolution of the HIV-1 envelope glycoprotein 120 association site of glycoprotein 41. J Biol Chem 278: 42149-42160.
29. Bellamy-McIntyre A K, Bar S, Ludlow L, Drummer H E, Poumbourios P (2010) Role for the disulfide-bonded region of human immunodeficiency virus type 1 gp41 in receptor-triggered activation of membrane fusion function. Biochem Biophys Res Commun 394: 904-908.
30. Abrahamyan L G, Markosyan R M, Moore J P, Cohen F S, Melikyan G B (2003) Human immunodeficiency virus type 1 env with an intersubunit disulfide bond engages coreceptors but requires bond reduction after engagement to induce fusion. J Virol 77: 5829-5836.
31. Binley J M, Cayanan C S, Wiley C, Schulke N, Olson W C, et al. (2003) Redox-triggered infection by disulfide-shackled human immunodeficiency virus type 1 pseudovirions. J Virol 77: 5678-5684.
32. Finzi A, Xiang S H, Pacheco B, Wang L, Haight J, et al. (2010) Topological layers in the HIV-1 gp120 inner domain regulate gp41 interaction and CD4-triggered conformational transitions. Mol Cell 37: 656-667.
33. Murray E J, Leaman D P, Pawa N, Perkins H, Pickford C, et al. (2010) A low-molecular-weight entry inhibitor of both CCR5- and CXCR4-tropic strains of human immunodeficiency virus type 1 targets a novel site on gp41. J Virol 84: 7288-7299.
34. Reitter J N, Means R E, Desrosiers R C (1998) A role for carbohydrates in immune evasion in AIDS. Nat Med 4: 679-684.
35. Wei X, Decker J M, Wang S, Hui H, Kappes J C, et al. (2003) Antibody neutralization and escape by HIV-1. Nature 422: 307-312.
36. Ching L K, Vlachogiannis G, Bosch K A, Stamatatos L (2008) The first hypervariable region of the gp120 Env glycoprotein defines the neutralizing susceptibility of heterologous human immunodeficiency virus type 1 isolates to neutralizing antibodies elicited by the SF162gp140 immunogen. J Virol 82: 949-956.
37. Kolchinsky P, Kiprilov E, Sodroski J (2001) Increased neutralization sensitivity of CD4-independent human immunodeficiency virus variants. J Virol 75: 2041-2050.
38. Krachmarov C P, Honnen W J, Kayman S C, Gorny M K, Zolla-Pazner S, et al. (2006) Factors determining the breadth and potency of neutralization by V3-specific human monoclonal antibodies derived from subjects infected with clade A or clade B strains of human immunodeficiency virus type 1. J Virol 80: 7127-7135.
39. Losman B, Bolmstedt A, Schonning K, Bjorndal A, Westin C, et al. (2001) Protection of neutralization epitopes in the V3 loop of oligomeric human immunodeficiency virus type 1 glycoprotein 120 by N-linked oligosaccharides in the V1 region. AIDS Res Hum Retroviruses 17: 1067-1076.
40. Li Y, Cleveland B, Klots I, Travis B, Richardson B A, et al. (2008) Removal of a single N-linked glycan in human immunodeficiency virus type 1 gp120 results in an enhanced ability to induce neutralizing antibody responses. J Virol 82: 638-651.
41. Pinter A, Honnen W J, He Y, Gorny M K, Zolla-Pazner S, et al. (2004) The V1/V2 domain of gp120 is a global regulator of the sensitivity of primary human immunodeficiency virus type 1 isolates to neutralization by antibodies commonly induced upon infection. J Virol 78: 5205-5215.
42. Sagar M, Wu X, Lee S, Overbaugh J (2006) Human immunodeficiency virus type 1 V1-V2 envelope loop sequences expand and add glycosylation sites over the course of infection, and these modifications affect antibody neutralization sensitivity. J Virol 80: 9586-9598.
43. van Gils M J, Bunnik E M, Boeser-Nunnink B D, Burger J A, Terlouw-Klein M, et al. (2011) Longer V1V2 region with increased number of potential N-linked glycosylation sites in the HIV-1 envelope glycoprotein protects against HIV-specific neutralizing antibodies. J Virol 85: 6986-6995.
44. van Gils M J, Edo-Matas D, Bowles E J, Burger J A, Stewart-Jones G B, et al. (2011) Evolution of human immunodeficiency virus type 1 in a patient with cross-reactive neutralizing activity in serum. J Virol 85: 8443-8448.
45. Day J R, Van Damme N, Guatelli J C (2006) The effect of the membrane-proximal tyrosine-based sorting signal of HIV-1 gp41 on viral infectivity depends on sequences within gp120. Virology 354: 316-327.
46. Crooks E T, Moore P L, Franti M, Cayanan C S, Zhu P, et al. (2007) A comparative immunogenicity study of HIV-1 virus-like particles bearing various forms of envelope proteins, particles bearing no envelope and soluble monomeric gp120. Virology 366: 245-262.
47. Crooks E T, Tong T, Osawa K, Binley J M (2011) Enzyme digests eliminate nonfunctional Env from HIV-1 particle surfaces, leaving native Env trimers intact and viral infectivity unaffected. J Virol 85: 5825-5839.
48. Karlsson G B, Gao F, Robinson J, Hahn B, Sodroski J (1996) Increased envelope spike density and stability are not required for the neutralization resistance of primary human immunodeficiency viruses. J Virol 70: 6136-6142.
49. Moore P L, Crooks E T, Porter L, Zhu P, Cayanan C S, et al. (2006) Nature of nonfunctional envelope proteins on the surface of human immunodeficiency virus type 1. J Virol 80: 2515-2528.
50. Tong T, Crooks E T, Osawa K, Binley J M (2012) HIV-1 virus-like particles bearing pure env trimers expose neutralizing epitopes but occlude nonneutralizing epitopes. J Virol 86: 3574-3587.
51. Farzan M, Mirzabekov T, Kolchinsky P, Wyatt R, Cayabyab M, et al. (1999) Tyrosine sulfation of the amino terminus of CCR5 facilitates HIV-1 entry. Cell 96: 667-676.
52. Lee B, Sharron M, Montaner L J, Weissman D, Doms R W (1999) Quantification of CD4, CCR5, and CXCR4 levels on lymphocyte subsets, dendritic cells, and differentially conditioned monocyte-derived macrophages. Proc Natl Acad Sci USA 96: 5215-5220.
53. Lee B, Sharron M, Blanpain C, Doranz B J, Vakili J, et al. (1999) Epitope mapping of CCR5 reveals multiple conformational states and distinct but overlapping structures involved in chemokine and coreceptor function. J Biol Chem 274: 9617-9626.
54. Zaitseva M, Peden K, Golding H (2003) HIV coreceptors: role of structure, posttranslational modifications, and internalization in viral-cell fusion and as targets for entry inhibitors. Biochim Biophys Acta 1614: 51-61.
55. Litwin V, Nagashima K A, Ryder A M, Chang C H, Carver J M, et al. (1996) Human immunodeficiency virus type 1 membrane fusion mediated by a laboratory-adapted strain and a primary isolate analyzed by resonance energy transfer. J Virol 70: 6437-6441.
56. Kuhmann S E, Platt E J, Kozak S L, Kabat D (2000) Cooperation of multiple CCR5 coreceptors is required for infections by human immunodeficiency virus type 1. J Virol 74: 7005-7015.
57. Chan D C, Chutkowski C T, Kim P S (1998) Evidence that a prominent cavity in the coiled coil of HIV type 1 gp41 is an attractive drug target. Proc Natl Acad Sci USA 95: 15613-15617.
58. Chen H, Xu X, Bishop A, Jones I M (2005) Reintroduction of the 2G12 epitope in an HIV-1 clade C gp120. AIDS 19: 833-835.
59. Duenas-Decamp M J, Clapham P R (2010) HIV-1 gp120 determinants proximal to the CD4 binding site shift protective glycans that are targeted by monoclonal antibody 2G12. J Virol 84: 9608-9612.
60. Gray E S, Moore P L, Pantophlet R A, Morris L (2007) N-linked glycan modifications in gp120 of human immunodeficiency virus type 1 subtype C render partial sensitivity to 2G12 antibody neutralization. J Virol 81: 10769-10776.
61. Sanders R W, Venturi M, Schiffner L, Kalyanaraman R, Katinger H, et al. (2002) The mannose-dependent epitope for neutralizing antibody 2G12 on human immunodeficiency virus type 1 glycoprotein gp120. J Virol 76: 7293-7305.
62. Scanlan C N, Pantophlet R, Wormald M R, Ollmann Saphire E, Stanfield R, et al. (2002) The broadly neutralizing anti-human immunodeficiency virus type 1 antibody 2G12 recognizes a cluster of alpha1-->2 mannose residues on the outer face of gp120. J Virol 76: 7306-7321.
63. Trkola A, Purtscher M, Muster T, Ballaun C, Buchacher A, et al. (1996) Human monoclonal antibody 2G12 defines a distinctive neutralization epitope on the gp120 glycoprotein of human immunodeficiency virus type 1. J Virol 70: 1100-1108.
64. Moore P L, Gray E S, Sheward D, Madiga M, Ranchobe N, et al. (2011) Potent and broad neutralization of HIV-1 subtype C by plasma antibodies targeting a quaternary epitope including residues in the V2 loop. J Virol 85: 3128-3141.
65. Walker L M, Phogat S K, Chan-Hui P Y, Wagner D, Phung P, et al. (2009) Broad and potent neutralizing antibodies from an African donor reveal a new HIV-1 vaccine target. Science 326: 285-289.
66. Roben P, Moore J P, Thali M, Sodroski J, Barbas C F, 3rd, et al. (1994) Recognition properties of a panel of human recombinant Fab fragments to the CD4 binding site of gp120 that show differing abilities to neutralize human immunodeficiency virus type 1. J Virol 68: 4821-4828.
67. Zhou T, Xu L, Dey B, Hessell A J, Van Ryk D, et al. (2007) Structural definition of a conserved neutralization epitope on HIV-1 gp120. Nature 445: 732-737.
68. Muster T, Steindl F, Purtscher M, Trkola A, Klima A, et al. (1993) A conserved neutralizing epitope on gp41 of human immunodeficiency virus type 1. J Virol 67: 6642-6647.
69. Ofek G, Tang M, Sambor A, Katinger H, Mascola J R, et al. (2004) Structure and mechanistic analysis of the anti-human immunodeficiency virus type 1 antibody 2F5 in complex with its gp41 epitope. J Virol 78: 10724-10737.
70. Song L, Sun Z Y, Coleman K E, Zwick M B, Gach J S, et al. (2009) Broadly neutralizing anti-HIV-1 antibodies disrupt a hinge-related function of gp41 at the membrane interface. Proc Natl Acad Sci USA 106: 9057-9062.
71. Meldal M, St Hilaire P M (1997) Synthetic methods of glycopeptide assembly, and biological analysis of glycopeptide products. Curr Opin Chem Biol 1: 552-563.
72. Imperiali B, O'Connor S E (1999) Effect of N-linked glycosylation on glycopeptide and glycoprotein structure. Curr Opin Chem Biol 3: 643-649.
73. Shental-Bechor D, Levy Y (2008) Effect of glycosylation on protein folding: a close look at thermodynamic stabilization. Proc Natl Acad Sci USA 105: 8256-8261.
74. Shental-Bechor D, Levy Y (2009) Folding of glycoproteins: toward understanding the biophysics of the glycosylation code. Curr Opin Struct Biol 19: 524-533.
75. York J, Nunberg J H (2004) Role of hydrophobic residues in the central ectodomain of gp41 in maintaining the association between human immunodeficiency virus type 1 envelope glycoprotein subunits gp120 and gp41. J Virol 78: 4921-4926.
76. Bellamy-McIntyre A K, Lay C S, Baar S, Maerz A L, Talbo G H, et al. (2007) Functional links between the fusion peptide-proximal polar segment and membrane-proximal region of human immunodeficiency virus gp41 in distinct phases of membrane fusion. J Biol Chem 282: 23104-23116.
77. Leavitt M, Park E J, Sidorov I A, Dimitrov D S, Quinnan G V, Jr. (2003) Concordant modulation of neutralization resistance and high infectivity of the primary human immunodeficiency virus type 1 M N strain and definition of a potential gp41 binding site in gp120. J Virol 77: 560-570.
78. Park E J, Quinnan G V, Jr. (1999) Both neutralization resistance and high infectivity phenotypes are caused by mutations of interacting residues in the human immunodeficiency virus type 1 gp41 leucine zipper and the gp120 receptor- and coreceptor-binding domains. J Virol 73: 5707-5713.
79. Liu L, Cimbro R, Lusso P, Berger E A (2011) Intraprotomer masking of third variable loop (V3) epitopes by the first and second variable loops (V1V2) within the native HIV-1 envelope glycoprotein trimer. Proc Natl Acad Sci USA 108: 20148-20153.
80. Musich T, Peters P J, Duenas-Decamp M J, Gonzalez-Perez M P, Robinson J, et al. (2011) A conserved determinant in the V1 loop of HIV-1 modulates the V3 loop to prime low CD4 use and macrophage infection. J Virol 85: 2397-2405.
81. Rusert P, Krarup A, Magnus C, Brandenberg O F, Weber J, et al. (2011) Interaction of the gp120 V1V2 loop with a neighboring gp120 unit shields the HIV envelope trimer against cross-neutralizing antibodies. J Exp Med 208: 1419-1433.
82. Saunders C J, McCaffrey R A, Zharkikh I, Kraft Z, Malenbaum S E, et al. (2005) The V1, V2, and V3 regions of the human immunodeficiency virus type 1 envelope differentially affect the viral phenotype in an isolate-dependent manner. J Virol 79: 9069-9080.
83. Haim H, Si Z, Madani N, Wang L, Courter J R, et al. (2009) Soluble CD4 and CD4-mimetic compounds inhibit HIV-1 infection by induction of a short-lived activated state. PLoS Pathog 5: e1000360.
84. Doores K J, Bonomelli C, Harvey D J, Vasiljevic S, Dwek R A, et al. (2010) Envelope glycans of immunodeficiency virions are almost entirely oligomannose antigens. Proc Natl Acad Sci USA 107: 13800-13805.
85. Kwong P D, Wyatt R, Sattentau Q J, Sodroski J, Hendrickson W A (2000) Oligomeric modeling and electrostatic analysis of the gp120 envelope glycoprotein of human immunodeficiency virus. J Virol 74: 1961-1972.
86. Leonard C K, Spellman M W, Riddle L, Harris R J, Thomas J N, et al. (1990) Assignment of intrachain disulfide bonds and characterization of potential glycosylation sites of the type 1 recombinant human immunodeficiency virus envelope glycoprotein (gp120) expressed in Chinese hamster ovary cells. J Biol Chem 265: 10373-10382.
87. Wyatt R, Kwong P D, Desjardins E, Sweet R W, Robinson J, et al. (1998) The antigenic structure of the HIV gp120 envelope glycoprotein. Nature 393: 705-711.
88. Bontjer I, Land A, Eggink D, Verkade E, Tuin K, et al. (2009) Optimization of human immunodeficiency virus type 1 envelope glycoproteins with V1/V2 deleted, using virus evolution. J Virol 83: 368-383.
89. Theodore T S, Englund G, Buckler-White A, Buckler C E, Martin M A, et al. (1996) Construction and characterization of a stable full-length macrophage-tropic HIV type 1 molecular clone that directs the production of high titers of progeny virions. AIDS Res Hum Retroviruses 12: 191-194.
90. Moss B, Elroy-Stein O, Mizukami T, Alexander W A, Fuerst T R (1990) New mammalian expression vectors. Nature 348: 91-92.
91. Poumbourios P, Wilson K A, Center R J, El Ahmar W, Kemp B E (1997) Human immunodeficiency virus type 1 envelope glycoprotein oligomerization requires the gp41 amphipathic alpha-helical/leucine zipper-like sequence. J Virol 71: 2041-2049.
92. Hill M K, Shehu-Xhilaga M, Campbell S M, Poumbourios P, Crowe S M, et al. (2003) The dimer initiation sequence stem-loop of human immunodeficiency virus type 1 is dispensable for viral replication in peripheral blood mononuclear cells. J Virol 77: 8329-8335.
93. Gao F, Morrison S G, Robertson D L, Thornton C L, Craig S, et al. (1996) Molecular cloning and analysis of functional envelope genes from human immunodeficiency virus type 1 sequence subtypes A through G. The WHO and NIAID Networks for HIV Isolation and Characterization. J Virol 70: 1651-1667.
94. Abacioglu Y H, Fouts T R, Laman J D, Claassen E, Pincus S H, et al. (1994) Epitope mapping and topology of baculovirus-expressed HIV-1 gp160 determined with a panel of murine monoclonal antibodies. AIDS Res Hum Retroviruses 10: 371-381.
95. Hatch W C, Tanaka K E, Calvelli T, Rashbaum W K, Kress Y, et al. (1992) Persistent productive HIV-1 infection of a CD4-human fetal thymocyte line. J Immunol 148: 3055-3061.
96. Page K A, Stearns S M, Littman D R (1992) Analysis of mutations in the V3 domain of gp160 that affect fusion and infectivity. J Virol 66: 524-533.
97. Chesebro B, Wehrly K, Nishio J, Perryman S (1992) Macrophage-tropic human immunodeficiency virus isolates from different patients exhibit unusual V3 envelope sequence homogeneity in comparison with T-cell-tropic isolates: definition of critical amino acids involved in cell tropism. J Virol 66: 6547-6554.
98. Toohey K, Wehrly K, Nishio J, Perryman S, Chesebro B (1995) Human immunodeficiency virus envelope V1 and V2 regions influence replication efficiency in macrophages by affecting virus spread. Virology 213: 70-79.
99. Takikawa S, Ishii K, Aizaki H, Suzuki T, Asakura H, et al. (2000) Cell fusion activity of hepatitis C virus envelope proteins. J Virol 74: 5066-5074.
100. Deng H, Liu R, Ellmeier W, Choe S, Unutmaz D, et al. (1996) Identification of a major co-receptor for primary isolates of HIV-1. Nature 381: 661-666.
101. Doranz B J, Lu Z H, Rucker J, Zhang T Y, Sharron M, et al. (1997) Two distinct CCR5 domains can mediate coreceptor usage by human immunodeficiency virus type 1. J Virol 71: 6305-6314.
102. Farzan M, Choe H, Vaca L, Martin K, Sun Y, et al. (1998) A tyrosine-rich region in the N terminus of CCR5 is important for human immunodeficiency virus type 1 entry and mediates an association between gp120 and CCR5. J Virol 72: 1160-1164.
103. Connor R I, Chen B K, Choe S, Landau N R (1995) Vpr is required for efficient replication of human immunodeficiency virus type-1 in mononuclear phagocytes. Virology 206: 935-944.
104. Bjorndal A, Deng H, Jansson M, Fiore J R, Colognesi C, et al. (1997) Coreceptor usage of primary human immunodeficiency virus type 1 isolates varies according to biological phenotype. J Virol 71: 7478-7487.
105. Dhillon A K, Donners H, Pantophlet R, Johnson W E, Decker J M, et al. (2007) Dissecting the neutralizing antibody specificities of broadly neutralizing sera from human immunodeficiency virus type 1-infected donors. J Virol 81: 6548-6562.
106. Sali A, Blundell T L (1993) Comparative protein modelling by satisfaction of spatial restraints. J Mol Biol 234: 779-815.
107. Bohne-Lang A, von der Lieth C W (2005) GlyProt: in silico glycosylation of proteins. Nucleic Acids Res 33: W214-219.
108. Lutteke T, Bohne-Lang A, Loss A, Goetz T, Frank M, et al. (2006) GLYCOSCIENCES.de: an Internet portal to support glycomics and glycobiology research. Glycobiology 16: 71R-81R.
109. Adachi A, Gendelman H E, Koenig S, Folks T, Willey R, et al. (1986) Production of acquired immunodeficiency syndrome-associated retrovirus in human and nonhuman cells transfected with an infectious molecular clone. J Virol 59: 284-291.
110. Pettersen E F, Goddard T D, Huang C C, Couch G S, Greenblatt D M, et al. (2004) UCSF Chimera-a visualization system for exploratory research and analysis. J Comput Chem 25: 1605-1612.

### BIBLIOGRAPHY 3

Altschul et al., Nucl. Acids Res. 25:3389, 1997
Ausubel (Ed) Current Protocols in Molecular Biology, 5^{th} Edition, John Wiley & Sons, Inc, N Y, 2002
Ausubel et al., Current Protocols in Molecular Biology John Wiley & Sons Inc, 1994-1998, Chapter 15
Beasley et al., Lancet, 2: 1099-102, 1983
Bonner et al., Eur. J. Biochem., 46:83, 1974
Bruno & Jacobson, Antimicrob Chemother 65: 1839-41, 2010
Burton et al., Proc Natl Acad Sci USA 108: 11181-6, 2011
Du A P, et al., J Mol Biol, 323:503-521, 2002
Erickson et al., Science, 249:527-533, 1990
Helseth E, et al., J Virol, 65:2119-2123, 1991
Hessell et al., PLOS pathog 5: e1000433, 2009
Hodgson, Bio/Technology, 9:19-21, 1991
Huang, et al., Science, 317:1930-1934, 2007
Huang et al., Nature 491: 406-12, 2012
Kurrek, Eur. J. Biochem., 270:1628-1644, 2003
Marmur et al., J. Mol. Biol., 5:109, 1962
Moldt et al., Proc Natl Acad Sci USA 109: 18921-5, 2012
Pancera et al., Proc Natl Acad Sci USA, 107:1166-1171, 2010
Remington's Pharmaceutical Sciences, 18^{th} Ed., 1990, Mack Publishing, Company, Easton, Pa., U.S.A.
Ruprecht et al., Vaccine. 21: 3370-3, 2003
Sambrook, Molecular Cloning: A Laboratory Manual, 3^{rd} Edition, CSHLP, CSH, N Y, 2001
Summerton et al., 7:187-195, 1997
Sun et al., Immunity, 28:52-63, 2008
Toma et al J Virol 85: 3872-80, 2011
Veazy et al., Nat Med 9: 343-6, 2003
Walker et al., Nature 477: 466-70, 2011
Walker et al., Science 326: 285-9, 2009
Wells, Methods Enzymol., 202:2699-2705, 1991

### BIBLIOGRAPHY EXAMPLE 4

1. Zhou T, Georgiev I, Wu X, Yang Z Y, Dai K, Finzi A, Kwon Y D, Scheid J F, Shi W, Xu L, Yang Y, Zhu J, Nussenzweig M C, Sodroski J, Shapiro L, Nabel G J, Mascola J R, Kwong P D. Structural basis for broad and potent neutralization of HIV-1 by antibody VRC01. Science 329:811-7 (2010)
2. Huang J, Ofek G, Laub L, Louder M K, Doria-Rose N A, Longo N S, Imamichi H, Bailer R T, Chakrabarti B, Sharma S K, Alam S M, Wang T, Yang Y, Zhang B, Migueles S A, Wyatt R, Haynes B F, Kwong P D, Mascola J R, Connors M. Broad and potent neutralization of HIV-1 by a gp41-specific human antibody. Nature 491:406-12 (2012)
3. Adachi A, Gendelman H E, Koenig S, Folks T, Willey R, Rabson A, Martin M A. Production of acquired immunodeficiency syndrome-associated retrovirus in human and nonhuman cells transfected with an infectious molecular clone. J Virol 59:284-91 (1986)
4. Lewis N, Williams J, Rekosh D, Hammarskjold M-L. Identification of a cis-acting element in human immunodeficiency virus type 2 (HIV-2) that is responsive to the HIV-1 rev and human T-cell leukemia virus types I and II rex proteins. J Virol 64:1690-7 (1990)
5. Chesebro B, Wehrly K, Nishio J, Perryman S. Macrophage-tropic human immunodeficiency virus isolates from different patients exhibit unusual V3 envelope sequence homogeneity in comparison with T-cell-tropic isolates: definition of critical amino acids involved in cell tropism. J Virol 66:6547-54 (1992)

1. A modified HIV envelope glycoprotein (Env) antigen or a lipid containing vehicle comprising same wherein the Env antigen comprises one of:
(i) a second site suppressor mutation in residue 674 of the membrane proximal ectodomain region (MPER) of HIV gp41;
(ii) a second site suppressor mutation which ablates a glycosylation site in the variable region (V1) region of gp120; or
(iii) a second site suppressor mutation ablating a glycosylation site in the V1 region of gp120 and a second site suppressor mutation in residue 674 of the MPER of HIV gp41.

2. The Env antigen or lipid vehicle of claim 1 comprising a gp120-gp41 association site mutation or a reversion or pseudoreversion mutation thereof in the disulfide bonded region (DSR) of gp120.

3. The Env antigen or lipid vehicle of claim 2 wherein the DSR mutation is at K601 and/or W596.

4. The Env antigen or lipid vehicle of claim 3 wherein the DSR mutation at K601 is selected from the group consisting of K601 D, K601H, K601N, K601Q and K601R.

5. The Env antigen or lipid vehicle of claim 3 wherein the DSR mutation at W596 is selected from the group consisting of W596I, W596L, W596H, W596M, W596Y, W596F and W596A.

6. The Env antigen or lipid vehicle of claim 1 wherein residue 674 is other than aspartic acid.

7. The Env antigen or lipid vehicle of claim 6 wherein residue 674 is glutamic acid.

8. The Env antigen or lipid vehicle of claim 1 wherein the glycosylation site mutation in V1 of HIV gp120 is ΔN¹³⁹INN or a mutation of asparagine(s), threonine(s) or serine(s) in other HIV strains that ablate analogous glycosylation sites.

9. The Env antigen or lipid vehicle of claim 1 wherein the glycosylation site mutation in V1 of HIV gp120 is T138N or a mutation of asparagine(s), threonine(s) or serine(s) in other HIV strains that ablate analogous glycosylation sites.

10. The lipid containing vehicle of claim 1 wherein the lipid vehicle is a human immunodeficiency virus like particle (HIVLP).

11. The lipid containing vehicle of claim 1 wherein the lipid vehicle is an enveloped virus or virus-like particle that is other than human immunodeficiency virus.

12. The lipid containing vehicle of claim 11 wherein the virus is selected from the group consisting of SIV, murine leukemia virus and other retroviruses, vesicular stomatitis virus, rabies virus, herpesvirus and hepadnavirus.

13. A modified Env antigen of claim 1 wherein the modified Env antigen comprises a mutation selected from the group consisting of ΔN¹³⁹INN/W596L/K601H/D674E, ΔN¹³⁹INN/W596L/K601D/D674E, ΔN¹³⁹INN/W596L/K601N/D674E, W596L/K601H/D674E, ΔN¹³⁹INN/W596L/K601H, T138N/W596L/K601H/D674E, T138N/ΔN¹³⁹INN, T138N, ΔN¹³⁹INN and a mutation of asparagine(s), threonine(s) or serine(s) in other HIV strains that ablate analogous glycosylation sites.

14. A HIVLP comprising the modified Env antigen of claim 13.

15. A virus or virus-like particle other than HIV comprising the modified Env antigen of claim 13.

16. A lipid vehicle of non-viral origin comprising the modified Env antigen of claim 13.

17. An isolated nucleic acid molecule encoding the modified Env antigen of claim 13.

18. A composition comprising the modified HIV Env antigen or lipid containing vehicle of claim 13 and a pharmaceutically or physiologically acceptable carrier or diluent.

19. A composition of claim 18 comprising an adjuvant.

20. (canceled)

21. (canceled)

22. (canceled)

23. A method of eliciting an immune response in a subject, the method comprising administering an effective amount of a composition according to claim 1 for a time and under conditions sufficient to elicit an immune response.

24. (canceled)

25. (canceled)

26. A method of immunising a subject against an HIV infection comprising administering a composition of claim 1 to the subject.

27. A method of treating or preventing an HIV infection in a subject comprising administering a composition of claim 1 to the subject for a time and under conditions sufficient to treat an HIV infection in a subject.
